(19) United States
(12) Patent Application Publication
(10) Pub. No.: US 2017/0044615 A1
(43) Pub. Date: Feb. 16, 2017

(54) METHODS OF PREDICTING MEDICALLY REFRACTIVE ULCERATIVE COLITIS (mrUC) REQUIRING COLECTOMY

(71) Applicant: Cedars-Sinai Medical Center, Los Angeles, CA (US)

(72) Inventors: Jerome I. Rotter, Los Angeles, CA (US); Kent D. Taylor, Ventura, CA (US); Stephan R. Targan, Santa Monica, CA (US); Talin Haritunians, Encino, CA (US); Dermot P. McGovern, Los Angeles, CA (US); Xiuqing Guo, Santa Monica, CA (US); Philip Fleshner, Los Angeles, CA (US)

(73) Assignee: Cedars-Sinai Medical Center, Los Angeles, CA (US)

(21) Appl. No.: 15/338,782

(22) Filed: Oct. 31, 2016

Related U.S. Application Data
(63) Continuation-in-part of application No. 13/140,874, filed on Nov. 16, 2011.
Application No. 13/140,874 is a continuation-in-part of application No. PCT/US2015/029101, filed on May 4, 2015.
(60) Provisional application No. 61/140,794, filed on Dec. 24, 2008.
(60) Provisional application No. 61/988,078, filed on May 2, 2014.

(51) Int. Cl. C12Q 1/68

(52) U.S. Cl. CPC C12Q 1/6883; C12Q 2600/156; C12Q 2600/158

(57) ABSTRACT

The present invention relates to methods of predicting the risk for colectomy in a subject with mrUC, by determining the presence or absence of one or more mrUC risk variants. Other embodiment, relate to methods of treating mrUC in a subject and a kit for prognostic use.

### CROSS-REFERENCE TO RELATED APPLICATIONS

This application is a continuation-in-part of U.S. Ser. No. 13/140,874 filed on Nov. 16, 2011, currently pending, which is a U.S. national stage application of PCT/US2009/069531 filed on Dec. 24, 2009, now expired, which claims priority to U.S. Ser. No. 61/140,794, filed on Dec. 24, 2008; this application is also a continuation-in-part of International Application No. PCT/US2015/029101, filed May 4, 2015, currently pending, which designated the U.S. and that International Application was published under PCT Article 21(2) in English, which claims priority under 35 U.S.C. §119(e) to U.S. Provisional Patent Application No. 61/988,078, filed May 2, 2014 now expired. The contents of all the aforementioned applications are herein incorporated by reference in their entirety.

### GOVERNMENT RIGHTS

This invention was made with government support under Contract Nos. DK046763 and DK062413 awarded by the National Institutes of Health. The government has certain rights in the invention.

### FIELD OF THE INVENTION

The invention relates generally to the fields of genetics and inflammatory disease, specifically medically refractive-UC (mrUC).

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

Thus, there is a need in the art to identify genes, allelic variants and/or haplotypes that may assist in determining the need for colectomy, diagnosing susceptibility or treatment for medically refractive ulcerative colitis (mrUC).

### SUMMARY OF THE INVENTION

Various embodiments of the present invention provide for a method of determining the need for colectomy in a subject with mrUC comprising: obtaining a sample from the subject; assaying the sample to detect the presence or absence of mrUC genetic risk variants, wherein the mrUC genetic risk variants are selected from the group consisting of SEQ ID NOs: 1-99; calculating a genetic risk score based on the detection of the mrUC genetic risk variants; determining that the subject has an increased likelihood of needing colectomy if the calculated genetic risk score is at the high end of the observed range and determining that the subject has a decreased likelihood of needing colectomy if the calculated genetic risk score is at the low end of the observed range. In various embodiments, the genetic risk score is obtained by calculating a total number of risk alleles for all the mrUC genetic risk variants assayed, wherein the risk allele for each mrUC genetic risk variant assayed is 0, 1 or 2.

Various other embodiments, further comprise obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected. In various embodiments, the number of mrUC genetic risk variants assayed is 46, the theoretical range is 0-92 and the observed range is 28-60. In various embodiments, the number of mrUC genetic risk variants assayed is 36, the theoretical range is 0-72 and the observed range is 16-38. Various other embodiments further comprise prescribing colectomy to subjects having a genetic risk score at the high end of the observed range. In various embodiments, time to colectomy is lower in a subject with a genetic risk score at the high end of the observed range and time to colectomy is higher in a subject with a genetic risk score at the low end of the observed range. In various embodiments, the time to colectomy is 10 to 70 months from detection.

Various embodiments of the present invention provide for a method of diagnosing susceptibility to mrUC in a subject, comprising: obtaining a sample from the subject; assaying the sample to detect the presence or absence of mrUC genetic risk variants, wherein the mrUC genetic risk variants are selected from the group consisting of SEQ ID NOs: 1-99; calculating a genetic risk score based on the detection of the mrUC genetic risk variants; and diagnosing susceptibility to mrUC based on the calculated risk score, wherein a subject has an increased susceptibility to mrUC if the calculated genetic risk score is at the high end of the observed range and a subject has a decreased susceptibility to mrUC if the calculated genetic risk score is at the low end of the observed range.

In various embodiments, the genetic risk score is obtained by calculating a total number of risk alleles for all the mrUC genetic risk variants assayed, wherein the risk allele for each mrUC genetic risk variant assayed is 0, 1 or 2.

Various other embodiments further comprise obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected. In various embodiments, an increase in the number of risk alleles detected signifies an increase in susceptibility to mrUC. In various embodiments, the number of mrUC genetic risk variants assayed is 46, the theoretical range is 0-92 and the observed range is 28-60. In various embodiments, the number of mrUC genetic risk variants assayed is 36, the theoretical range is 0-72 and the observed range is 16-38.

Various other embodiments further comprise prescribing colectomy to subjects diagnosed with a susceptibility for mrUC and have a genetic risk score at the high end of the observed range. In various embodiments, the time to colectomy is lower in a subject with a genetic risk score at the high end of the observed range and the time to colectomy is higher in a subject with a genetic risk score at the low end of the observed range. In various embodiments, the time to colectomy is 10 to 70 months from detection.

In various other embodiments of the present invention provides for a method of treating mrUC in a subject, comprising: obtaining a sample from the subject; assaying the sample to detect the presence or absence of mrUC genetic risk variants, wherein the mrUC genetic risk variants are selected from the group consisting of SEQ ID NOs: 1-99; calculating a genetic risk score based on the detection of the mrUC genetic risk variants; diagnosing susceptibility to mrUC based on the calculated risk score, wherein a subject has an increased susceptibility to mrUC if the calculated genetic risk score is high and a subject has a decreased susceptibility to mrUC if the calculated genetic risk score is low; and prescribing colectomy to the subject with an increased susceptibility to mrUC.

In various embodiments, the genetic risk score is obtained by calculating a total number of risk alleles for all the mrUC genetic risk variants assayed, wherein the risk allele for each mrUC genetic risk variant assayed is 0, 1 or 2.

Various other embodiments further comprise obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected. In various embodiments, an increase in the number of risk alleles detected signifies an increase in susceptibility to mrUC. In various embodiments, the number of mrUC genetic risk variants assayed is 46, the theoretical range is 0-92 and the observed range is 28-60. In various embodiments, the number of mrUC genetic risk variants assayed is 36, the theoretical range is 0-72 and the observed range is 16-38.

In various embodiments, the treatment is colectomy and is prescribed to subjects diagnosed with a susceptibility for mrUC and have a genetic risk score at the high end of the observed range. In various other embodiments, the time to colectomy is lower in a subject with a genetic risk score at the high end of the observed range and the time to colectomy is higher in a subject with a genetic risk score at the low end of the observed range. In various embodiments, the time to colectomy is 10 to 70 months from detection.

Various embodiments of the present invention provide for a kit for prognostic use, comprising: a single prognostic panel comprising one or more medically refractive ulcerative colitis (mrUC) genetic risk variants described in SEQ ID NOs: 1-99.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with an embodiment herein, a schematic describing mrUC vs. non-mrUC survival analysis and risk modeling.

FIGS. 2A-2B depicts, in accordance with an embodiment herein, FIG. 2A) Higher risk score categories are associated with mrUC (χ2 test for trend p<2.2×10−16). Risk score (observed range: 28-60) was divided into quarters: scores 28-38 (risk-A); scores 39-45 (risk-B); scores 46-52 (risk-C); and scores 53-60 (risk-D). Percentage of mrUC is noted, along with the total number of UC subjects in each risk category. FIG. 2B) Higher risk score categories are associated with an earlier progression to colectomy at 24 and 60 months. Risk score was divided into quarters: scores 28-38 (risk-A); scores 39-45 (risk-B); scores 46-52 (risk-C); and scores 53-60 (risk-D). At 24 months, risk of colectomy was 3.1%, 19.1% and 62% for risk-B, -C, and -D, respectively. Risk of colectomy at 60 months increased to 8.3%, 48.4%, 84% for risk-B, -C, and -D, respectively. Total number of UC subjects in each risk category is given.

FIG. 3 depicts, in accordance with an embodiment herein, serology data demonstrating an association of mrUC with Cbir1, ASCA, OmpC and I2 antibody quartile sum in mrUC and non-mrUC subjects.

FIG. 4 depicts, in accordance with an embodiment herein, single SNP association tested with logistic regression analysis in mrUC and non-mrUC subjects.

FIG. 5 depicts, in accordance with an embodiment herein, a schematic describing mr UC vs. Non-mrUC survival analysis and risk modeling for mrUC.

FIG. 6 depicts, in accordance with an embodiment herein, a chart with the top 36 associated SNPs from Analysis I and II, referenced herein.

FIG. 7 depicts, in accordance with an embodiment herein, higher risk score association with mrUC.

FIG. 8 depicts, in accordance with an embodiment herein, higher risk score association with earlier progression to colectomy.

FIG. 9 depicts, in accordance with an embodiment herein, higher risk score exhibits a shorter overall median time to colectomy.

FIG. 10 depicts, in accordance with an embodiment herein, potential clinical utility of the association of a higher risk score with earlier progression to colectomy.

FIG. 11 depicts, in accordance with an embodiment herein, role for major histocompatibility (MHC) in UC severity in mrUC versus controls.

FIG. 12 depicts, in accordance with an embodiment herein, single SNP association tested with regression analysis in mrUC versus controls.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed, J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3^{rd} ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“IBD” as used herein is an abbreviation of inflammatory bowel disease.

“CD” as used herein is an abbreviation of Crohn's Disease.

“UC” as used herein is an abbreviation of ulcerative colitis.

“GWAS” as used herein is an abbreviation of genome wide association study.

“mrUC” as used herein is defined as ulcerative colitis with symptoms uncontrolled by medical therapy. Also referred to as mr-UC.

As used herein, the term “mrUC genetic risk variant” refers to genetic variants, or SNPs, that have an association with the mrUC, or ulcerative colitis requiring colectomy, phenotype.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

A “Risk Score” as used herein is a calculated number, obtained by adding/totaling the total number of risk alleles for all the mrUC genetic risk variants assayed. The risk allele for each mrUC genetic risk variant assayed is 0, 1 or 2. For example, when analyzing a patient for 5 mrUC genetic risk variants, the detected risk alleles may be 1, 0, 1, 2, and 1, which when added will give the patient a risk score of 5 (1+0+1+2+1=5). The risk score, based on analyzed mrUC genetic risk variants, is calculated in other patients and the cumulative risk scores for all patients analyzed provide an observed range as discussed below.

“Risk Group” as used herein refers to a subset of patients who fall within the same category for colectomy risk based on the detected mrUC risk variants in the subject's biological sample.

“Treatment”, as used herein refers to both therapeutic treatment and prophylactic or preventative measures, wherein the object is to prevent or slow down (lessen) the targeted pathologic condition, prevent the pathologic condition, pursue or obtain good overall survival, or lower the chances of the individual developing the condition even if the treatment is ultimately unsuccessful. Those in need of treatment include those already with the condition as well as those prone to have the condition or those in whom the condition is to be prevented. Examples of mrUC treatment include, but are not limited to, active surveillance, observation, surgical intervention (such as colectomy), drug therapy (anti-inflammatory and/or immune system suppressor drugs), targeted therapy to genes known to be involved in mrUC, such as, but not limited to those referenced herein and/or a combination thereof.

“Time to colectomy” as used herein refers to the amount of time between the determination that a subject had an increased likelihood of needing colectomy and actually undergoing colectomy. In one embodiments, the subject has a reduced time to colectomy (for example: 0-6 months, 6 months-1 year, 1-2 years or 2-3 years) if the subject has a high risk score. In another embodiment, the subject has an increased time to colectomy (for example, 3-4 years, 4-5 years or more) if the subject has a low risk score.

“Theoretical range” as used herein refers to the minimum and maximum number of risk alleles possible based on the number of mrUC genetic risk variants assayed. For example, if 46 genetic risk variants are analyzed, the theoretical range is 0-92, where 0 is the minimum number of risk alleles and 92 (46×2 alleles) is the maximum number of risk alleles.

“Observed range” as used herein refers to the minimum and maximum risk score, which is based on the risk alleles detected for the patient cohort, as described above. For example, an observed range of 28-60, obtained when analyzing the 46 genetic risk variants, results in a minimum of 28 and a maximum of 60.

“High end” of an observed range as used herein refers to a genetic risk score that is within for example, 10-15 points of the maximum observed range.

“Low end” of an observed range as used herein refers to a genetic score that is within for example, 10-15 points of the minimum observed range.

Acute severe ulcerative colitis (UC) remains a significant clinical challenge and the ability to predict, at an early stage, those individuals at risk of colectomy for medically refractory UC (mrUC) would be a major clinical advance. As disclosed herein, the inventors used a genome-wide association study (GWAS) in a well characterized cohort of UC patients to identify genetic variation that contributes to mrUC. A GWAS comparing 324 mrUC patients with 537 Non-mrUC patients was analyzed using logistic regression and Cox proportional hazards methods. In addition, the mrUC patients were compared with 2601 healthy controls.

As further disclosed herein, mrUC was associated with more extensive disease (p=2.7×10−6) and a positive family history of UC (p=0.004). A risk score based on the combination of 46 SNPs associated with mrUC explained 48% of the variance for colectomy risk in the cohort. Risk scores divided into quarters showed the risk of colectomy to be 0%, 17%, 74% and 100% in the four groups. Comparison of the mrUC subjects with healthy controls confirmed the contribution of the major histocompatibility complex to severe UC (peak association: rs17207986, p=1.4×10−16) and provided genome-wide suggestive association at the TNFSF15 (TL1A) locus (peak association: rs11554257, p=1.4×10−6). A SNP-based risk scoring system, identified herein by GWAS analyses, can provide a useful adjunct to clinical parameters for predicting natural history in UC. Furthermore, discovery of genetic processes underlying disease severity can help to identify pathways for novel therapeutic intervention in severe UC.

### Determining the Need for Colectomy

Various embodiments of the present invention provide for a method of determining the need for colectomy in a subject with mrUC comprising: obtaining a sample from the subject; assaying the sample to detect the presence or absence of mrUC genetic risk variants, wherein the mrUC genetic risk variants are selected from the group consisting of SEQ ID NOs: 1-99; calculating a genetic risk score based on the detection of the mrUC genetic risk variants; determining that the subject has an increased likelihood of needing colectomy if the calculated genetic risk score is at the high end of the observed range and determining that the subject has a decreased likelihood of needing colectomy if the calculated genetic risk score is at the low end of the observed range. In various embodiments, the genetic risk score is obtained by calculating a total number of risk alleles for all the mrUC genetic risk variants assayed, wherein the risk allele for each mrUC genetic risk variant assayed is 0, 1 or 2.

Various other embodiments, further comprise obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected. In various embodiments, the number of mrUC genetic risk variants assayed is 46, the theoretical range is 0-92 and the observed range is 28-60. In various embodiments, the number of mrUC genetic risk variants assayed is 36, the theoretical range is 0-72 and the observed range is 16-38.

Various other embodiments further comprise prescribing colectomy to subjects having a genetic risk score at the high end of the observed range. In various embodiments, time to colectomy is lower in a subject with a genetic risk score at the high end of the observed range and time to colectomy is higher in a subject with a genetic risk score at the low end of the observed range. In various embodiments, the time to colectomy is 10 to 70 months from detection.

### Diagnosing Susceptibility

Various embodiments of the present invention provide for a method of diagnosing susceptibility to mrUC in a subject, comprising: obtaining a sample from the subject; assaying the sample to detect the presence or absence of mrUC genetic risk variants, wherein the mrUC genetic risk variants are selected from the group consisting of SEQ ID NOs: 1-99; calculating a genetic risk score based on the detection of the mrUC genetic risk variants; and diagnosing susceptibility to mrUC based on the calculated risk score, wherein a subject has an increased susceptibility to mrUC if the calculated genetic risk score is at the high end of the observed range and a subject has a decreased susceptibility to mrUC if the calculated genetic risk score is at the low end of the observed range.

In various embodiments, the genetic risk score is obtained by calculating a total number of risk alleles for all the mrUC genetic risk variants assayed, wherein the risk allele for each mrUC genetic risk variant assayed is 0, 1 or 2

Various other embodiments further comprise obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected. In various embodiments, an increase in the number of risk alleles detected signifies an increase in susceptibility to mrUC. In various embodiments, the number of mrUC genetic risk variants assayed is 46, the theoretical range is 0-92 and the observed range is 28-60. In various embodiments, the number of mrUC genetic risk variants assayed is 36, the theoretical range is 0-72 and the observed range is 16-38.

Various other embodiments further comprise prescribing colectomy to subjects diagnosed with a susceptibility for mrUC and have a genetic risk score at the high end of the observed range. In various embodiments, the time to colectomy is lower in a subject with a genetic risk score at the high end of the observed range and the time to colectomy is higher in a subject with a genetic risk score at the low end of the observed range. In various embodiments, the time to colectomy is 10 to 70 months from detection.

### Treatment

In various other embodiments of the present invention provides for a method of treating mrUC in a subject, comprising: obtaining a sample from the subject; assaying the sample to detect the presence or absence of mrUC genetic risk variants, wherein the mrUC genetic risk variants are selected from the group consisting of SEQ ID NOs: 1-99; calculating a genetic risk score based on the detection of the mrUC genetic risk variants; diagnosing susceptibility to mrUC based on the calculated risk score, wherein a subject has an increased susceptibility to mrUC if the calculated genetic risk score is high and a subject has a decreased susceptibility to mrUC if the calculated genetic risk score is low; and prescribing colectomy to the subject with an increased susceptibility to mrUC.

In various embodiments, the genetic risk score is obtained by calculating a total number of risk alleles for all the mrUC genetic risk variants assayed, wherein the risk allele for each mrUC genetic risk variant assayed is 0, 1 or 2.

Various other embodiments further comprise obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected. In various embodiments, an increase in the number of risk alleles detected signifies an increase in susceptibility to mrUC. In various embodiments, the number of mrUC genetic risk variants assayed is 46, the theoretical range is 0-92 and the observed range is 28-60. In various embodiments, the number of mrUC genetic risk variants assayed is 36, the theoretical range is 0-72 and the observed range is 16-38.

In various embodiments, the number of mrUC genetic risk variants assays is 1-10, 10-20, 20-30, 30-40, 40-50, 50-60, 60-70, 70-80, 80-90, or 90-99.

In various embodiments, the treatment is colectomy and is prescribed to subjects diagnosed with a susceptibility for mrUC and have a genetic risk score at the high end of the observed range. In various other embodiments, the time to colectomy is lower in a subject with a genetic risk score at the high end of the observed range and the time to colectomy is higher in a subject with a genetic risk score at the low end of the observed range. In various embodiments, the time to colectomy is 10 to 70 months from detection.

Those in need of treatment include those already with the condition as well as those prone to have the condition or those in whom the condition is to be prevented. Examples of mrUC treatment include, but are not limited to, active surveillance, observation, surgical intervention (such as colectomy), drug therapy (anti-inflammatory and/or immune system suppressor drugs), and targeted therapy, directed to genes known to be involved in IBD, such as, but not limited to those referenced herein and/or a combination thereof. Targeted therapy can consist of administering a composition(s) that will modify gene regulation by inhibiting or inducing the target gene expression and/or activity of the gene.

### Kits

Various embodiments of the present invention provide for a kit for prognostic use, comprising: a single prognostic panel comprising one or more medically refractive ulcerative colitis (mrUC) genetic risk variants described in SEQ ID NOs: 1-99. In various embodiments, one or more medically refractive ulcerative colitis (mrUC) genetic risk variants is 1-10, 10-20, 20-30, 30-40, 40-50, 50-60, 60-70, 70-80, 80-90, 90-99 medically refractive ulcerative colitis (mrUC) genetic risk variants.

The present invention is directed to a kit to predict the risk for colectomy, susceptibility to mrUC and/or treatment of mrUC. The kit is useful for practicing the inventive method of determining risk for colectomy in a subject, diagnosing susceptibility to mrUC in a subject and/or treatment of a subject. The kit is an assemblage of materials or components, including at least one of the inventive compositions. In various embodiments, the kit contains a composition including a drug that targets genes known to be involved in mrUC, such as the mrUC genetic risk variants, for treatment of mrUC, as described above. Thus, in some embodiments the kit contains a composition including primers and probes to genetic risk alleles and/or drugs useful in targeting those genetic risk alleles.

The exact nature of the components configured in the inventive kit depends on its intended purpose. For example, some embodiments are configured for the purpose of treating mrUC. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome. Optionally, the kit also contains other useful components, such as, primers, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

A variety of methods can be used to determine the presence or absence of an mrUC genetic risk variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well-known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261(1993); White et al., Genomics 12:301-306 (1992)).

The technique of single strand conformational, polymorphism (SSCP) also may be used to detect the presence or absence of a SNP and/or a haplotype (see Hayashi, K., Methods Applic.1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention may be practiced using one or any combination of the well-known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Overall

Acute severe ulcerative colitis (UC) remains a significant clinical challenge and the ability to predict, at an early stage, those individuals at risk of colectomy for medically refractory UC (mrUC) would be a major clinical advance. As disclosed herein, the inventors used a genome-wide association study (GWAS) in a well characterized cohort of UC patients to identify genetic variation that contributes to mrUC. A GWAS comparing 324 mrUC patients with 537 Non-mrUC patients was analyzed using logistic regression and Cox proportional hazards methods. In addition, the mrUC patients were compared with 2601 healthy controls.

As further disclosed herein, mrUC was associated with more extensive disease (p=2.7×10−6) and a positive family history of UC (p=0.004). A risk score based on the combination of 46 SNPs associated with mrUC explained 48% of the variance for colectomy risk in the cohort. Risk scores divided into quarters showed the risk of colectomy to be 0%, 17%, 74% and 100% in the four groups. Comparison of the mrUC subjects with healthy controls confirmed the contribution of the major histocompatibility complex to severe UC (peak association: rs17207986 (SEQ ID NO: 47), p=1.4×10−16) and provided genome-wide suggestive association at the INFSF15 (TL1A) locus (peak association: rs11554257 (SEQ ID NO: 48), p=1.4×10−6). A SNP-based risk scoring system, identified herein by GWAS analyses, can provide a useful adjunct to clinical parameters for predicting natural history in UC. Furthermore, discovery of genetic processes underlying disease severity can identify pathways for novel therapeutic intervention in severe UC.

### Example 2

### UC Cases

Ulcerative Colitis (UC) subjects (n=929) were recruited at Cedars Sinai-Medical Center Inflammatory Bowel Disease Center following informed consent after approval by the Institutional Review Board. UC diagnosis was based on standard criteria 31. UC subjects requiring colectomy for severe disease refractory to medical therapies (including intravenous corticosteroids, cyclosporine, and biologic therapies) were classified as medically refractory UC (mrUC). Subjects requiring colectomy where the indication was for treatment of cancer/dysplasia, in addition to subjects not requiring colectomy, were classified as Non-mrUC. Subjects who required colectomy for mrUC and were subsequently found to have evidence of dysplasia or carcinoma in the resected colon were classified as mrUC (n=3). For the mrUC cohort, time from diagnosis to date of colectomy was collected; time from diagnosis to last follow-up visit was obtained for the Non-mrUC cohort. Samples which did not genotype successfully (n=16), exhibited gender mismatch (n=9) or cryptic relatedness (n=13), or were considered outliers by principal components analysis (n=30) were excluded. Following these measures, 861 UC subjects (mrUC n=324; Non-mrUC n=537) were included in the analyses.

### Example 3

### Non-IBD Controls

Controls were obtained from the Cardiovascular Health Study (CHS), a population-based cohort study of risk factors for cardiovascular disease and stroke in adults 65 years of age or older, recruited at four field centers. 5,201 predominantly Caucasian individuals were recruited in 1989-1990 from random samples of Medicare eligibility lists, followed by an additional 687 African-Americans recruited in 1992-1993 (total n=5,888). CHS was approved by the Institutional Review Board at each recruitment site, and subjects provided informed consent for the use of their genetic information. A total of 2,601 Caucasian non-IBD control subjects who underwent GWAS were included in these analyses. African-American CHS participants were excluded from analysis due to insufficient number of ethnically-matched cases.

### Example 4

### Genotyping

All genotyping was performed at the Medical Genetics Institute at Cedars-Sinai Medical Center using Infinium technology (Illumina, San Diego, Calif.). UC cases were genotyped with either the HumanCNV370-Quad or Human610-Quad platform; controls were genotyped with the HumanCNV370-Duo platform. Identity-by-descent was used to exclude related individuals (Pi-hat scores >0.5; PLINK). Average genotyping rate among cases and controls retained in the analysis was >99.8% and >99.2%, respectively. Single nucleotide polymorphisms (SNPs) were excluded based on: test of Hardy-Weinberg Equilibrium p<10-3; SNP failure rate >10%; MAF<3%; SNPs not found in dbSNP Build 129. 313,720 SNPs passed quality control measures and were common in all data sets.

### Example 5

### Population Stratification

Principal components analysis (Eigenstrat as implemented in Helix Tree) (Golden Helix, Bozeman, Mont.) was conducted to examine population stratification. Extreme outliers, defined as subjects with more than two standard deviations (SD) away from the distribution of the rest of the samples for any component, were removed. All African-American participants identified by principal components analysis were excluded from these analyses. Genetic heterogeneity following correction for population sub-structure was low, with estimated genomic inflation factors (λGC) of 1.04 and 1.06 for mrUC vs. Non-mrUC, and mrUC cases vs. Non-IBD controls analyses, respectively.

### Example 6

### mrUC Vs. Non-mrUC

### Survival Analysis and Risk Modeling

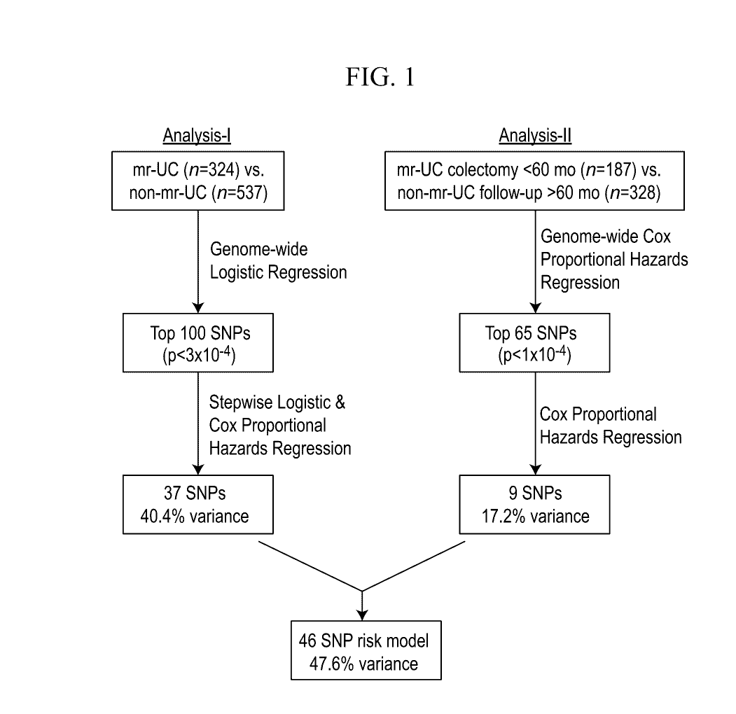

Single marker association analysis of mrUC vs. Non-mrUC (analysis-I) was performed using a logistic regression model correcting for population stratification using 20 principal components as covariates (PLINK v1.06). Association between medically refractory disease (mrUC) and the top 100 SNPs together (as determined by the lowest corrected p-values) from analysis-I were tested using a stepwise logistic regression model. SNPs were further analyzed by Cox proportional hazards regression utilizing time-to information, as described for UC cases (using the step and glm, and coxph functions, respectively, in R v2.9.0). 37 SNPs identified with logistic regression p<0.05 and Cox proportional hazards p<0.1 were retained in the risk model. The 100 SNPs (p<3×10−4) evaluated from analysis-I are listed herein (Table 1). A genome-wide Cox proportional hazards regression analysis (analysis-II) was then performed on a subset of the UC cohort (mrUC subjects with colectomy <60 months, n=187; Non-mrUC followed up >60 months, n=328) correcting for population stratification using two principal components as covariates (PLINK). The top 65 SNPs (8 of which overlap with the 100 SNPs from analysis-I above) were tested together (using coxph function in R). The 65 SNPs (p<1×10−4) from analysis-II are listed herein (Table 2). From these 65 SNPs, 9 SNPs were identified (p<3×10−4) and combined with the 37 SNPs from analysis-I to identify a final risk model consisting of 46 SNPs (see FIG. 1 for schematic; Table 3). A genetic risk score was calculated from the total number of risk alleles (0, 1, or 2) across all 46 risk SNPs (theoretical range: 0-92). Risk score (observed range: 28-60) was divided into quarters: scores 28-38 (risk-A); scores 39-45 (risk-B); scores 46-52 (risk-C); and scores 53-60 (risk-D). Receiver operating characteristic (ROC) curve and area under the ROC curve (AUC) were calculated using R software v2.9.0, including packages survival and survivalROC 39-41. Sensitivity and specificity curves, positive and negative predictive values, positive (sensitivity/1-specificity) and negative likelihood ratio (1-sensitivity/specificity) were all calculated using the R package ROCR 42. 1000-fold replication of 10-fold cross-validation was implemented to validate the fitted logistic regression model. Mean sensitivity and specificity were then re-calculated using the 1000 replicated samples. Bootstrap method with 1000-fold replication was utilized for estimating variability of hazard ratio estimated from the Cox regression model. The hazard ratio in survival analysis is the effect of an explanatory variable on the hazard or risk of an event.

TABLE 1
Top 100 SNPs from Analysis I

| | | | Minor | | Odds | | |
|---|---|---|---|---|---|---|---|
| Chr* | SNP | Position* | allele | P-value | Ratio | Stat | Loci** |
| 1 | rs260970 | 39323829 | G | 2.38E−04 | 1.594 | 3.675 | MACF1 \| 643910 \| |
| 1 | rs6697447 | 54219515 | A | 2.06E−04 | 0.4481 | −3.712 | HSPB11 \| YIPF1 \| |
| | | | | | | | C1orf83 \| |
| 1 | rs746503 | 54842574 | A | 2.27E−04 | 1.475 | 3.687 | ACOT11 \| FAM151A \| |
| | | | | | | | C1orf175 \| 645442 \| |
| 1 | rs2275612 | 95140004 | A | 1.87E−04 | 1.838 | 3.736 | CNN3 \| SLC44A3 \| |
| | | | | | | | 646896 \| 729970 \| |
| 1 | rs4847368 | 95149626 | G | 7.75E−05 | 1.976 | 3.952 | CNN3 \| 646896 \| 729970 \| |
| 1 | rs2298162 | 95221621 | G | 2.85E−04 | 0.6668 | −3.628 | CNN3 \| ALG14 \| |
| 1 | rs7550055 | 157045388 | C | 1.35E−04 | 1.571 | 3.818 | MNDA \| OR6N2 \| |
| | | | | | | | OR10AA1P \| OR6K4P \| |
| | | | | | | | OR6N1 \| OR6K3 \| |
| | | | | | | | OR6K5P \| 646377 \| |
| 1 | rs7367845 | 224512151 | A | 2.55E−04 | 1.475 | 3.657 | ACBD3 \| LIN9 \| |
| 1 | rs9286999 | 224561138 | A | 1.61E−04 | 1.491 | 3.773 | LIN9 \| 100128832 \| |
| 2 | rs892878 | 137588330 | A | 2.78E−04 | 0.6726 | −3.635 | THSD7B \| |
| 2 | rs1560579 | 137592445 | A | 2.67E−04 | 0.6326 | −3.646 | THSD7B \| |
| 2 | rs9287461 | 137593668 | G | 8.64E−05 | 0.6156 | −3.926 | THSD7B \| |
| 2 | rs958323 | 137606935 | G | 1.85E−04 | 0.6408 | −3.738 | THSD7B \| |
| 2 | rs1483148 | 142036240 | C | 1.59E−04 | 0.6225 | −3.777 | LRP1B \| |
| 2 | rs1448901 | 206961885 | G | 2.13E−05 | 1.673 | 4.251 | ADAM23 \| |
| 2 | rs7565690 | 224105705 | A | 2.10E−04 | 0.5414 | −3.706 | |
| 2 | rs4487082 | 229432205 | G | 2.03E−04 | 0.4091 | −3.716 | |
| 3 | rs403961 | 1575422 | G | 2.09E−04 | 1.495 | 3.708 | |
| 3 | rs924022 | 65824936 | G | 1.98E−04 | 0.5251 | −3.721 | MAGI1 \| |
| 3 | rs10511119 | 79943297 | G | 2.87E−04 | 0.6796 | −3.627 | |
| 3 | rs9682694 | 114378369 | G | 1.47E−04 | 1.549 | 3.797 | BOC \| |
| 3 | rs4839637 | 144422638 | A | 2.15E−04 | 1.501 | 3.701 | |
| 4 | rs2286461 | 15572771 | G | 1.86E−04 | 1.487 | 3.738 | PROM1 \| FGFBP1 \| |
| | | | | | | | FGFBP2 \| 100130067 \| |
| 4 | rs12650313 | 41401850 | A | 2.57E−04 | 1.877 | 3.655 | LIMCH1 \| 100128654 \| |
| 4 | rs1546318 | 79168396 | C | 9.31E−05 | 1.932 | 3.908 | FRAS1 \| 391670 \| |
| | | | | | | | 100128297 \| |
| 4 | rs1393644 | 79175731 | C | 9.19E−05 | 1.93 | 3.911 | FRAS1 \| 391670 \| |
| | | | | | | | 100128297 \| |
| 4 | rs1399403 | 108639264 | A | 1.60E−04 | 1.538 | 3.775 | |
| 4 | rs11098020 | 110122894 | A | 1.90E−04 | 1.777 | 3.732 | COL25A1 \| |
| 4 | rs7675371 | 116049368 | A | 2.64E−04 | 0.58 | −3.648 | NDST4 \| |
| 4 | rs6821443 | 122566710 | C | 2.90E−04 | 0.669 | −3.624 | QRFPR \| 391692 \| |
| | | | | | | | 729109 \| 729112 \| |
| 5 | rs3846599 | 10308821 | A | 1.06E−04 | 1.517 | 3.877 | MARCH6 \| CCT5 \| |
| | | | | | | | FAM173B \| MIR378 \| |
| 5 | rs12652447 | 15727635 | A | 3.86E−05 | 1.524 | 4.115 | FBXL7 \| |
| 5 | rs6596684 | 105972832 | G | 1.13E−04 | 1.53 | 3.861 | 345571 \| |
| 5 | rs6870711 | 126446993 | A | 1.98E−04 | 2.404 | 3.722 | MARCH3 \| 401207 \| |
| 6 | rs1536242 | 6876009 | A | 2.71E−04 | 0.62 | −3.642 | |
| 6 | rs17207986 | 32187545 | G | 4.71E−05 | 2.297 | 4.069 | ATF6B \| RNF5 \| PPT2 \| |
| | | | | | | | EGFL8 \| 653033 \| |
| 6 | rs3734263 | 34946407 | G | 2.37E−04 | 0.4474 | −3.676 | TAF11 \| ANKS1A \| |
| | | | | | | | UHRF1BP1 \| |
| 6 | rs9470224 | 36248614 | A | 1.70E−04 | 1.843 | 3.759 | BRPF3 \| PNPLA1 \| |
| 6 | rs777649 | 68925053 | A | 1.10E−05 | 1.618 | 4.396 | 642902 \| |
| 6 | rs3777505 | 75937343 | A | 1.04E−04 | 2.526 | 3.881 | COL12A1 \| |
| 6 | rs6908055 | 107015887 | G | 1.13E−04 | 1.67 | 3.861 | AIM1 \| |
| 6 | rs9400010 | 107027893 | G | 1.44E−04 | 1.56 | 3.802 | AIM1 \| |
| 7 | rs11760555 | 12563060 | G | 2.92E−04 | 0.6713 | −3.622 | SCIN \| |
| 7 | rs4722456 | 25338225 | A | 9.50E−05 | 0.6644 | −3.903 | 100131016 \| |
| 7 | rs13244827 | 131735522 | G | 1.68E−04 | 0.3415 | −3.763 | PLXNA4 \| |
| 7 | rs851685 | 147125736 | A | 2.25E−04 | 1.546 | 3.69 | CNTNAP2 \| |
| 8 | rs2978310 | 2701133 | A | 2.68E−04 | 1.496 | 3.644 | |
| 8 | rs1471474 | 76216530 | A | 1.12E−04 | 0.6541 | −3.864 | |
| 8 | rs6994721 | 76220268 | G | 7.63E−05 | 0.6521 | −3.956 | |
| 8 | rs4734754 | 105347978 | C | 1.57E−04 | 0.6496 | −3.78 | TM7SF4 \| |
| 8 | rs4734757 | 105355266 | A | 1.95E−04 | 0.6537 | −3.725 | TM7SF4 \| |
| 8 | rs263241 | 131931602 | A | 2.44E−04 | 1.456 | 3.669 | ADCY8 \| |
| 9 | rs7861972 | 6759692 | G | 2.34E−04 | 0.473 | −3.68 | JMJD2C \| SNRPEL1 \| |
| 9 | rs11265961 | 91638884 | A | 2.12E−05 | 1.586 | 4.252 | |
| 9 | rs2145929 | 116621761 | G | 2.44E−04 | 1.634 | 3.668 | TNFSF15 \| 645266 \| |
| | | | | | | | 100129633 \| |
| 9 | rs10817934 | 118589872 | A | 1.39E−04 | 0.6364 | −3.811 | ASTN2 \| |
| 10 | rs3793792 | 50520169 | A | 2.94E−04 | 1.481 | 3.62 | CHAT \| C10orf53 \| |
| 10 | rs518525 | 129520863 | A | 1.65E−04 | 1.488 | 3.767 | PTPRE \| 387720 \| |
| 11 | rs2403456 | 11134390 | A | 1.08E−04 | 2.413 | 3.872 | |
| 11 | rs4356200 | 37489093 | A | 6.49E−05 | 0.6231 | −3.994 | 100132895 \| |
| 11 | rs1461898 | 37546808 | A | 5.46E−05 | 0.5881 | −4.035 | |
| 11 | rs1075025 | 37582318 | G | 2.38E−04 | 0.6141 | −3.675 | |
| 11 | rs767289 | 37624038 | G | 3.99E−05 | 0.6274 | −4.108 | 100132631 \| |
| 11 | rs10837504 | 40775682 | A | 1.50E−04 | 1.649 | 3.792 | |
| 11 | rs6591765 | 62674829 | A | 7.84E−05 | 0.6468 | −3.949 | SLC22A24 \| |
| 11 | rs7949840 | 62741273 | G | 6.75E−05 | 0.6511 | −3.985 | SLC22A24 \| SLC22A25 |
| | | | | | | | \| SLC22A10 \| |
| 11 | rs11231409 | 62741444 | G | 7.46E−05 | 0.6527 | −3.961 | SLC22A24 \| SLC22A25 |
| | | | | | | | \| SLC22A10 \| |
| 12 | rs887357 | 3344906 | C | 2.16E−04 | 0.5947 | −3.699 | 643119 \| 728230 \| |
| | | | | | | | 100128253 \| |
| 12 | rs970063 | 13424516 | A | 1.87E−04 | 1.48 | 3.736 | C12orf36 \| |
| 12 | rs12581840 | 19725418 | G | 1.05E−04 | 0.6566 | −3.88 | |
| 12 | rs526058 | 24326688 | A | 6.51E−05 | 0.6009 | −3.994 | |
| 12 | rs1144720 | 32157518 | G | 2.82E−05 | 1.571 | 4.188 | BICD1 \| 729457 \| |
| 12 | rs1613650 | 32169080 | G | 2.65E−04 | 1.507 | 3.647 | BICD1 \| 729457 \| |
| 12 | rs2683471 | 32171607 | A | 2.14E−04 | 1.518 | 3.702 | BICD1 \| 729457 \| |
| 14 | rs1956388 | 28202628 | A | 1.32E−04 | 0.672 | −3.822 | |
| 14 | rs11156667 | 30906111 | A | 1.75E−05 | 0.6296 | −4.294 | HEATR5A \| 728852 \| |
| 14 | rs9323262 | 53863964 | A | 2.69E−04 | 0.5639 | −3.644 | CDKN3 \| |
| 14 | rs10133064 | 85844148 | A | 2.03E−04 | 0.4618 | −3.715 | |
| 14 | rs35795554 | 85854768 | C | 2.56E−04 | 0.4743 | −3.656 | |
| 15 | rs7172534 | 24855745 | G | 2.52E−04 | 1.503 | 3.661 | GABRG3 \| |
| 15 | rs965355 | 57847116 | G | 1.04E−04 | 1.584 | 3.881 | BNIP2 \| 100130107 \| |
| 15 | rs965353 | 57847498 | G | 1.38E−04 | 1.569 | 3.811 | BNIP2 \| 100130107 \| |
| 15 | rs10519111 | 59169989 | G | 1.08E−04 | 1.719 | 3.872 | RORA \| |
| 15 | rs990422 | 98377291 | A | 2.97E−04 | 1.533 | 3.618 | ADAMTS17 \| |
| 15 | rs1585933 | 98403238 | G | 6.14E−05 | 1.605 | 4.007 | ADAMTS17 \| |
| 16 | rs305087 | 84539747 | G | 4.72E−05 | 0.5364 | −4.069 | 100131952 \| |
| 17 | rs759258 | 52483547 | A | 2.39E−04 | 1.561 | 3.674 | AKAP1 \| |
| 18 | rs3848490 | 2326366 | A | 2.97E−04 | 1.48 | 3.618 | |
| 18 | rs8088744 | 64685792 | A | 1.72E−04 | 0.6688 | −3.757 | CCDC102B \| |
| 19 | rs2967682 | 8644532 | A | 9.96E−05 | 0.6538 | −3.892 | MYO1F \| ADAMTS10 \| |
| | | | | | | | OR2Z1 \| 390880 \| |
| 19 | rs11085825 | 12868458 | A | 6.05E−05 | 0.6309 | −4.011 | |
| 19 | rs2293683 | 12900284 | A | 8.35E−05 | 0.636 | −3.934 | |
| 19 | rs1010222 | 12909608 | A | 1.52E−04 | 0.6469 | −3.788 | CALR \| DNASE2 \| |
| | | | | | | | FARSA \| NFIX \| |
| | | | | | | | RAD23A \| KLF1 \| |
| | | | | | | | DAND5 \| SYCE2 \| |
| 19 | rs4808408 | 15881376 | G | 1.47E−05 | 0.628 | −4.334 | CYP4F2 \| CYP4F11 \| |
| | | | | | | | OR10H4 \| 440511 \| |
| | | | | | | | 646596 \| 729645 \| |
| | | | | | | | 729654 \| |
| 19 | rs12459140 | 15882888 | G | 1.52E−05 | 0.6287 | −4.326 | CYP4F2 \| CYP4F11 \| |
| | | | | | | | OR10H4 \| 440511 \| |
| | | | | | | | 646596 \| 729645 \| |
| | | | | | | | 729654 \| |
| 20 | rs6034134 | 15182479 | A | 1.00E−04 | 0.6518 | −3.89 | MACROD2 \| |
| 20 | rs10485594 | 19772393 | A | 6.91E−05 | 2.126 | 3.979 | RIN2 \| 644298 \| |
| 20 | rs6059101 | 31182314 | A | 6.54E−05 | 0.601 | −3.993 | C20orf71 \| C20orf70 \| |
| | | | | | | | 317716 \| 391242 \| |
| 20 | rs6059104 | 31185354 | A | 1.55E−04 | 0.6545 | −3.784 | C20orf71 \| C20orf70 \| |
| | | | | | | | 317716 \| 391242 \| |
| 22 | rs909502 | 47050966 | A | 2.65E−04 | 1.483 | 3.647 | |

TABLE 2
Top 65 SNPs from Analysis II

| Chr | SNP | Position129 | Minor_allele | P_value | Loci |
|---|---|---|---|---|---|
| 1 | rs1392127 | 55788503 | A | 5.25E−05 | 400754\| |
| 1 | rs2298162 | 95221621 | G | 1.58E−05 | CNN3\|ALG14\| |
| 2 | rs1448901 | 206961885 | G | 5.08E−05 | ADAM23\| |
| 2 | rs3791994 | 207718164 | A | 2.66E−05 | KLF7\| |
| 3 | rs900569 | 41834977 | G | 1.91E−05 | ULK4\| |
| 3 | rs6796430 | 73950170 | A | 5.77E−05 | |
| 3 | rs9843732 | 135505746 | G | 6.00E−05 | RYK\| |
| 4 | rs1013300 | 13204657 | G | 9.52E−05 | NKX3-2\|BOD1L\|285548\| |
| 4 | rs1491262 | 13301398 | A | 9.20E−05 | BOD1L\|644868\| |
| 4 | rs17476066 | 15461202 | G | 4.87E−05 | CD38\| |
| 4 | rs2608816 | 39103204 | G | 9.90E−05 | RFC1\|LIAS\|KLB\|642885\| |
| 4 | rs6811556 | 180521808 | A | 9.46E−06 | |
| 5 | rs6892546 | 5873530 | G | 7.03E−05 | |
| 5 | rs4571457 | 107862146 | G | 7.40E−06 | |
| 6 | rs9468256 | 28003483 | A | 8.81E−05 | HIST1H1B\|HIST1H2AK\| |
| | | | | | HIST1H2AM\|HIST1H3I\| |
| | | | | | HIST1H3J\|HIST1H4K\|HIST1H4L\| |
| | | | | | OR2B6\|OR2W6P\|OR2W4P\| |
| | | | | | OR2B2\| |
| 6 | rs2116984 | 28040741 | A | 4.39E−05 | HIST1H1B\|HIST1H2AM\| |
| | | | | | HIST1H3I\|HIST1H3J\|HIST1H4L\| |
| | | | | | OR2B6\|OR2W4P\|OR2W2P\| |
| | | | | | OR2B7P\|OR2B2\| |
| 6 | rs1012411 | 30440534 | C | 8.56E−05 | HCG18\|646491\|100129192\| |
| 6 | rs9501030 | 30907378 | A | 4.27E−06 | DDR1\|GTF2H4\|IER3\|FLOT1\| |
| | | | | | VARS2\|646553\|MIR588\| |
| 6 | rs9295930 | 30957801 | A | 9.38E−05 | DDR1\|GTF2H4\|VARS2\|DPCR1\| |
| | | | | | 646553\|646570\|MIR588\|729778\| |
| 6 | rs10947114 | 31010160 | A | 7.87E−05 | VARS2\|DPCR1\|SFTA2\|MUC21\| |
| | | | | | 646563\|646570\|MIR588\|729778\| |
| | | | | | 729792\| |
| 6 | rs537160 | 32024379 | A | 1.44E−05 | CFB\|C2\|C4A\|C4B\|NEU1\| |
| | | | | | SKIV2L\|RDBP\|STK19\|EHMT2\| |
| | | | | | SLC44A4\|ZBTB12\| |
| 6 | rs4151657 | 32025519 | G | 5.17E−05 | CFB\|C2\|C4A\|C4B\|NEU1\| |
| | | | | | SKIV2L\|RDBP\|STK19\|EHMT2\| |
| | | | | | SLC44A4\|ZBTB12\| |
| 6 | rs630379 | 32030233 | T | 3.11E−05 | CFB\|C2\|C4A\|C4B\|NEU1\| |
| | | | | | SKIV2L\|RDBP\|STK19\|EHMT2\| |
| | | | | | SLC44A4\|ZBTB12\| |
| 6 | rs9267845 | 32301676 | T | 4.04E−05 | |
| 6 | rs6910071 | 32390832 | G | 1.84E−05 | NOTCH4\|C6orf10\| |
| 6 | rs2894253 | 32453518 | C | 1.95E−05 | HLA-DRA\|C6orf10\|646668\| |
| 6 | rs34330585 | 32498681 | G | 4.30E−05 | HLA-DRA\|C6orf10\|BTNL2\| |
| | | | | | 646668\| |
| 7 | rs2158767 | 17019311 | G | 7.51E−05 | |
| 7 | rs1178163 | 18754088 | A | 5.55E−06 | HDAC9\| |
| 7 | rs11764116 | 18766938 | A | 2.26E−06 | HDAC9\| |
| 7 | rs2389992 | 18903915 | G | 1.30E−05 | HDAC9\| |
| 7 | rs929351 | 81695829 | C | 8.33E−06 | CACNA2D1\| |
| 8 | rs2980654 | 6480608 | G | 6.62E−05 | |
| 8 | rs6474026 | 56956047 | G | 7.63E−05 | LYN\| |
| 8 | rs2383847 | 73166848 | G | 8.52E−05 | TRPA1\| |
| 8 | rs2954870 | 75995015 | G | 8.76E−05 | CRISPLD1\| |
| 9 | rs3118292 | 25133480 | G | 2.83E−05 | |
| 9 | rs1331501 | 92432152 | G | 9.55E−05 | DIRAS2\|340515\| |
| 11 | rs1783983 | 57177356 | A | 8.95E−05 | |
| 11 | rs1031232 | 57628857 | G | 6.93E−05 | |
| 11 | rs6591765 | 62674829 | A | 4.62E−05 | SLC22A24\| |
| 11 | rs7949840 | 62741273 | G | 6.67E−05 | SLC22A24\|SLC22A25\|SLC22A10\| |
| 11 | rs11231409 | 62741444 | G | 8.18E−05 | SLC22A24\|SLC22A25\|SLC22A10\| |
| 12 | rs2098102 | 50268 39 | A | 9.72E−05 | KCNA5\|390282\| |
| 12 | rs906724 | 126243850 | A | 5.15E−05 | 100132564\| |
| 13 | rs4769736 | 28876751 | G | 7.36E−05 | KIAA0774\| |
| 13 | rs10507842 | 75481600 | G | 8.61E−05 | |
| 13 | rs7319358 | 78448935 | A | 3.34E−06 | |
| 14 | rs1956388 | 28202628 | A | 2.23E−06 | |
| 14 | rs2179891 | 28215603 | A | 6.90E−05 | FOXG1\|C14orf23\| |
| 14 | rs8020281 | 94436179 | A | 7.28E−05 | |
| 16 | rs1421069 | 51755435 | G | 8.00E−05 | CHD9\| |
| 16 | rs2388011 | 51770920 | G | 9.60E−05 | CHD9\| |
| 16 | rs3815548 | 51879235 | G | 9.94E−05 | CHD9\|441770\|100132875\| |
| 16 | rs1424203 | 59355718 | A | 3.55E−05 | |
| 17 | rs9898519 | 24865310 | G | 3.62E−05 | TAOK1\|TP53I13\|ANKRD13B\| |
| | | | | | 645942\| |
| 17 | rs3744624 | 24885455 | G | 1.48E−05 | TAOK1\|TP53I13\|ANKRD13B\| |
| | | | | | 645942\| |
| 18 | rs669924 | 38725419 | A | 7.79E−05 | RIT2\| |
| 19 | rs2116941 | 10195443 | A | 3.25E−05 | |
| 20 | rs755171 | 31176251 | G | 9.70E−05 | C20orf71\|C20orf70\|317716\| |
| | | | | | 391242\| |
| 20 | rs6059101 | 31182314 | A | 3.71E−06 | C20orf71\|C20orf70\|317716\| |
| | | | | | 391242\| |
| 20 | rs6059104 | 31185354 | A | 9.73E−05 | C20orf71\|C20orf70\|317716\| |
| | | | | | 391242\| |
| 21 | rs2831462 | 28370367 | A | 4.17E−05 | |
| 22 | rs916234 | 46165555 | G | 5.77E−05 | |
| 22 | rs2051594 | 47259952 | A | 5.43E−05 | 643266\|643325\| |

TABLE 3
46 SNPs associated with the risk model for mrUC

| Chr | SNP | Position129 | SEQ ID NO | Risk_allele | Loci |
|---|---|---|---|---|---|
| 1 | rs746503 | 54842574 | 1 | A | ACOT11\|FAM151A\|C1orf175\| |
| | | | | | 645442\| |
| 1 | rs2275612 | 95140004 | 2 | A | CNN3\|SLC44A3\|646896\|729970\| |
| 1 | rs7550055 | 157045388 | 3 | C | MNDA\|OR6N2\|OR2AQ1P\| |
| | | | | | OR10AA1P\|OR6K4P\|OR6N1\| |
| | | | | | OR6K3\|OR6K5P\|646377\| |
| 1 | rs7367845 | 224512151 | 4 | A | ACBD3\|MIXL1\|LIN9\|100128832\| |
| 2 | rs1448901 | 206961885 | 5 | G | ADAM23\|100132849\| |
| 2 | rs4487082 | 229432205 | 6 | A | 2q36.3 |
| 3 | rs900569 | 41834977 | 7 | G | ULK4\| |
| 3 | rs924022 | 65824936 | 8 | A | MAGI1\| |
| 3 | rs9843732 | 135505746 | 9 | G | RYK\| |
| 4 | rs2286461 | 15572771 | 10 | G | PROM1\|FGFBP1\|FGFBP2\| |
| | | | | | 100130067\| |
| 4 | rs12650313 | 41401850 | 11 | A | LIMCH1\|100128654\| |
| 4 | rs1399403 | 108639264 | 12 | A | 4q25 |
| 4 | rs7675371 | 116049368 | 13 | G | NDST4\| |
| 5 | rs3846599 | 10308821 | 14 | A | MARCH6\|CCT5\|FAM173B\|MIR378\| |
| 5 | rs6596684 | 105972832 | 15 | G | 345571\| |
| 6 | rs1536242 | 6876009 | 16 | G | 6p25.1 |
| 6 | rs17207986 | 32187545 | 17 | G | ATF6B\|RNF5\|PPT2\|EGFL8\|653033\| |
| 6 | rs777649 | 68925053 | 18 | A | 642902\| |
| 7 | rs11764116 | 18766938 | 19 | A | HDAC9\| |
| 7 | rs4722456 | 25338225 | 20 | G | 100131016\| |
| 7 | rs929351 | 81695829 | 21 | A | CACNA2D1\| |
| 8 | rs2980654 | 6480608 | 22 | G | ANGPT2\|AGPAT5\|MCPH1\| |
| | | | | | 100131112\|100132301\| |
| 8 | rs6994721 | 76220268 | 23 | A | 8q21.11 |
| 8 | rs4734754 | 105347978 | 24 | A | RIMS2\|TM7SF4\| |
| 9 | rs7861972 | 6759692 | 25 | A | JMJD2C\|SNRPEL1\| |
| 9 | rs3118292 | 25133480 | 26 | G | 9p21.3 |
| 9 | rs10817934 | 118589872 | 27 | G | ASTN2\| |
| 11 | rs2403456 | 11134390 | 28 | A | 11p15.3 |
| 11 | rs1461898 | 37546808 | 29 | G | 100132895\|100132631\| |
| 11 | rs6591765 | 62674829 | 30 | G | SLC22A24\|SLC22A25\|SLC22A10\| |
| 12 | rs887357 | 3344906 | 31 | A | 643119\|728230\|100128253\| |
| 12 | rs526058 | 24326688 | 32 | G | 12p12.1 |
| 13 | rs7319358 | 78448935 | 33 | A | 13q31.1 |
| 14 | rs1956388 | 28202628 | 34 | G | 14q12 |
| 14 | rs11156667 | 30906111 | 35 | G | GPR33\|HEATR5A\|NUBPL\| |
| | | | | | C14orf126\|728852\| |
| 14 | rs10133064 | 85844148 | 36 | C | 14q31.3 |
| 14 | rs8020281 | 94436179 | 37 | A | 14q32.13 |
| 15 | rs965353 | 57847498 | 38 | G | BNIP2\|100130107\|GTF2A2\| |
| 16 | rs305087 | 84539747 | 39 | A | 100131952\| |
| 17 | rs759258 | 52483547 | 40 | A | AKAP1\| |
| 19 | rs2967682 | 8644532 | 41 | C | MYO1F\|ADAMTS10\|OR2Z1\| |
| | | | | | 390880\| |
| 19 | rs2293683 | 12900284 | 42 | G | |
| 20 | rs6034134 | 15182479 | 43 | C | MACROD2\| |
| 20 | rs10485594 | 19772393 | 44 | A | RIN2\|644298\| |
| 20 | rs6059104 | 31185354 | 45 | G | PLUNC\|C20orf71\|C20orf70\| |
| | | | | | C20orf186\|317716\|391242\| |
| 21 | rs2831462 | 28370367 | 46 | A | 21q21.3 |

### Example 7

### mrUC Vs. Non-IBD Controls

### Regression Analysis

Single marker analysis of genome-wide data for mrUC cases vs. Non-IBD Caucasian controls from CHS (analysis-III) was performed as before, using logistic regression correcting for 20 principal components (PLINK).

### Example 8

### UC Subject Demographics

Complete temporal data was available on 861 UC subjects (mrUC n=324; Non-mrUC n=537). The demographic data of the cohort is summarized herein. The inventors observed no differences in gender, median age of onset of disease, and smoking status between the medically refractory and Non-mrUC subjects. There was a significant difference in our median disease duration (p=7.4×10−9), with the time from diagnosis to last follow-up in the Non-mrUC cohort nearly double the time from diagnosis to colectomy in our mrUC subjects. Additionally, there was a significantly higher incidence of disease that extended proximal to the splenic flexure (p=2.7×10−6) in the mrUC group when compared to Non-mrUC, consistent with previously published data. The inventors identified a novel association between a family history (first or second degree relative) of UC and the development of mrUC (p=0.004).

### Example 9

### Forty-Six SNP Risk Model is Associated with mrUC and Predicts Earlier Progression to Colectomy

The inventors performed a GWAS on 324 mrUC and 537 Non-mrUC subjects. Results of this analysis (analysis-I) are given herein and discussed below. Following identification of single markers associated with mrUC, the inventors proceeded to a multivariate approach. Beginning with the top 100 results from analysis-I (p<3×10−4), the inventors performed a stepwise logistic regression and identified 64 SNPs (p<0.05) that together were associated with medically refractory disease (mrUC) and were carried forward to survival analysis. Of these 64 SNPs, 37 SNPs remained (Cox proportional hazards regression p<0.1; OR 1.2-1.8), which explained 40% of the variance for mrUC. In order to elucidate the maximum discrimination, i.e. greatest percentage of the variance, the inventors further performed a genome-wide Cox proportional hazards regression analysis (analysis-II) on a subset of the UC cohort to identify SNPs involved in earlier progression to colectomy. Testing together the top 65 SNPs from this analysis (p<1×10−4), the inventors identified nine SNPs with Cox proportional hazards p<3×10−4 (individual OR ranged from 1.4-1.6), explaining 17% of the variance. Beginning with the previously identified 37 risk SNP model, these 9 SNPs were added sequentially to the model. This analysis resulted in the final risk model of 46 SNPs (OR for MR-UC for each individual SNP ranged from 1.2-1.9), which explained 48% of the variance for colectomy in the mrUC cohort.

The inventors calculated a genetic risk score from the total number of risk alleles across all 46 risk SNPs (theoretical range: 0-92). The observed risk score ranged from 28-60, and was significantly associated with mrUC (logistic regression and Cox proportional hazards pvalues <10-16). An ROC curve using this risk score gave an AUC of 0.91. The sensitivity of the fitted model for mrUC was 0.793, with a specificity of 0.858. Using 1000 replicates of the 10-fold cross-validation data, they obtained a mean sensitivity of 0.789 (SD=0.0067) and mean specificity of 0.859 (SD=0.002). This indicates that the fitted model was robust and only ˜0.4% over-fitting was observed. The hazard ratio was estimated to be 1.313 from the Cox regression model. 1000 replicates of bootstrapped samples gave an estimated hazard ratio of 1.314 (SD=0.017) (Table 4).

TABLE 4

| Sensitivity/Specificity | Sensitivity 0.793 |
|---|---|
| (cut-off = .5) | Specificity 0.858 |
| Hazard Ratio: | 1.313 |

1000 times of 10 fold Cross-Validation data sets with logistic regression

| Variable | N | Mean | Std Dev | Minimum | Maximum |
|---|---|---|---|---|---|
| Sensitivity | 1000 | 0.789 | 0.0067 | 0.758 | 0.793 |
| Specificity | 1000 | 0.859 | 0.0021 | 0.858 | 0.87 |

1000 fold Bootstrapping:

| N | Mean | Std Dev | Minimum | Maximum |
|---|---|---|---|---|
| 1000 | 1.314 | 0.017 | 1.269 | 1.372 |

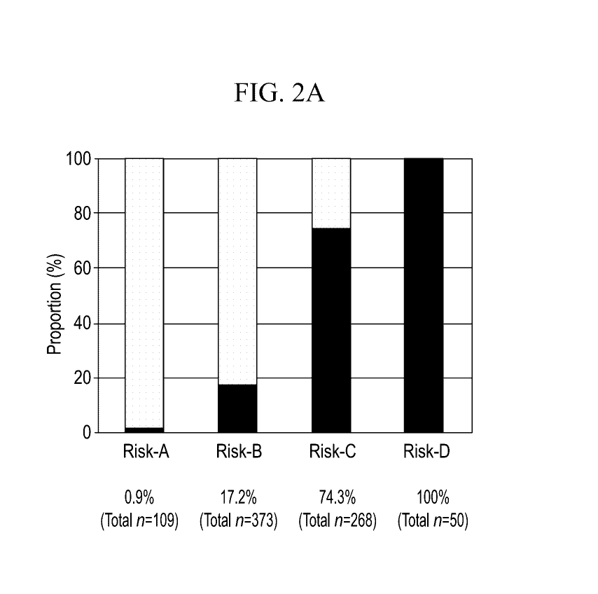

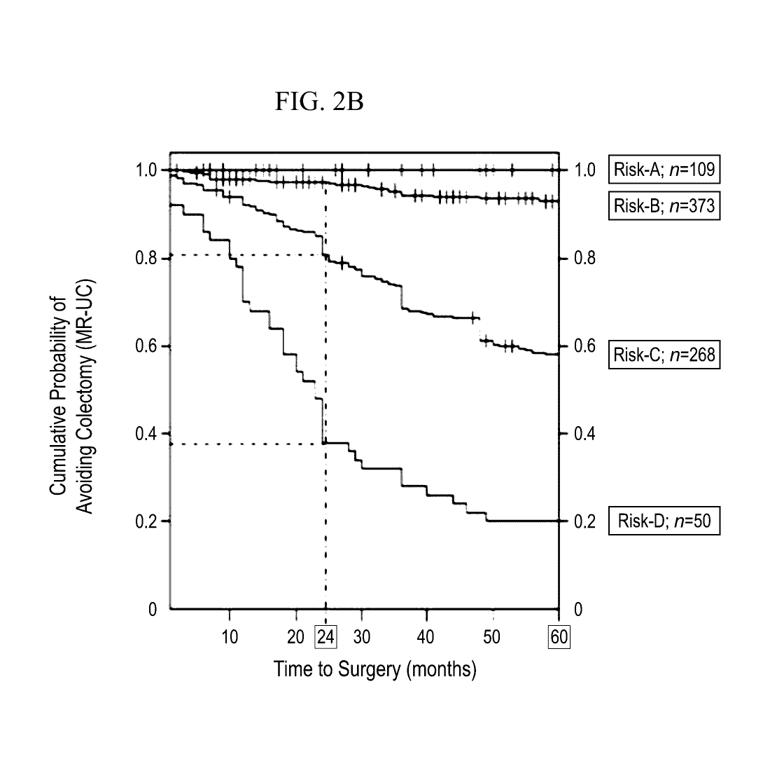

Based on the genetic risk scores, the inventors grouped the UC cohort into four risk categories; less than 1% of cases in the lowest risk category (risk-A) were mrUC and the percentage of mrUC increased to ˜17%, ˜74% and 100% in risk-B, -C and -D groups, respectively (χ2 test for trend p<2.2×10−16; FIG. 2A). The median time to colectomy for risk-C and -D categories was 72 months and 23 months, respectively. Progression to colectomy within 2 and 5 years of diagnosis may be more clinically relevant and while no individuals in the risk-A category had undergone colectomy at either 2 or 5 years after diagnosis, the respective incidence of mrUC at 2 years for risk groups-B, -C and -D was 3.1%, 19.1%, and 62%, respectively, and at 5 years was 8.3%, 50%, and 80%, respectively (FIG. 2B). At five years from diagnosis, either the total risk score (AUC 0.86) or the risk category (AUC 0.82) are able to predict patients that will require surgery. The operating characteristics of the risk score system are shown herein. A score of 44 and 47 can be used to generate a test with a sensitivity (to exclude a diagnosis of colectomy) and specificity (to include a diagnosis) of over 90%, respectively. Loci corresponding to the 46 SNPs in the risk model include several compelling candidate genes for UC severity and suggest potential biological pathways for further avenues of study. As each risk SNP contributes modestly to the overall risk of mrUC (OR 1.2-1.9), this work supports the paradigm that a group of SNPs, identified by GWAS and combined together may account for a large proportion of the genetic contribution to a complex phenotype (48% of the variance for risk in this study) to provide a risk score with clinical utility.

### Example 10

### MHC Region and TLIA (TNFSF15) Contribute to UC Severity

Association analyses between 324 UC subjects with mrUC and 2,601 population matched controls confirmed a major contribution of the major histocompatibility (MHC) on chromosome 6p to the development of severe UC (analysis-III; Table 5). Ten SNPs in MHC reached a priori defined level of genome-wide significance (p≦5×10−7; 87 SNPs with p<1×10−3), with peak association at rs17207986 (SEQ ID NO: 47; p=1.4×10−16). Three SNPs on chromosome 9q, a locus which contains the known IBD susceptibility gene TNFSF15 (TL1A), achieved genome-wide suggestive significance (p<5×10−5), with the most significant association seen at rs11554257 (SEQ ID NO: 48; p=1.4×10−6).

TABLE 5
MHC region associated SNPs

| | | | Minor | | Odds | | |
|---|---|---|---|---|---|---|---|
| Chr * | SNP | Position* | Allele | P-value | Ratio | Stat | Loci** |
| 6 | rs3132679 | 30183822 | A | 9.40E−05 | 0.5327 | −3.906 | TRIM31 \| RNF39 \| |
| | | | | | | | TRIM15 \| TRIM40 \| |
| 6 | rs9468692 | 30227869 | A | 6.64E−04 | 1.767 | 3.404 | TRIM10 \| TRIM31 \| |
| | | | | | | | RNF39 \| TRIM15 \| |
| | | | | | | | TRIM40 \| |
| 6 | rs1012411 | 30440534 | C | 2.98E−04 | 1.526 | 3.617 | HCG18 \| 646491 \| |
| | | | | | | | 100129192 \| |
| 6 | rs2040450 | 30442318 | A | 3.54E−04 | 1.65 | 3.572 | HCG18 \| 646491 \| |
| | | | | | | | 100129192 \| 100129772 \| |
| 6 | rs2524211 | 30458639 | A | 4.75E−04 | 1.612 | 3.495 | HCG18 \| 646491 \| |
| | | | | | | | 646520 \| 100129192 \| |
| | | | | | | | 100129772 \| |
| 6 | rs9261761 | 30480966 | A | 2.28E−04 | 0.5646 | −3.685 | HLA-E \| MICC \| HCG18 |
| | | | | | | | \| 646520 \| 100129192 \| |
| | | | | | | | 100129772 \| |
| 6 | rs9261817 | 30486580 | C | 2.33E−04 | 0.565 | −3.68 | HLA-E \| MICC \| HCG18 |
| | | | | | | | \| 646520 \| 100129192 \| |
| | | | | | | | 100129772 \| |
| 6 | rs9261821 | 30487053 | G | 2.46E−04 | 0.5663 | −3.667 | HLA-E \| MICC \| HCG18 |
| | | | | | | | \| 646520 \| 100129192 \| |
| | | | | | | | 100129772 \| |
| 6 | rs9261846 | 30490419 | G | 2.42E−04 | 0.5662 | −3.671 | HLA-E \| MICC \| HCG18 |
| | | | | | | | \| 646520 \| 100129192 \| |
| | | | | | | | 100129772 \| |
| 6 | rs9261847 | 30490626 | C | 4.44E−04 | 0.5793 | −3.512 | HLA-E \| MICC \| HCG18 |
| | | | | | | | \| 646520 \| 100129192 \| |
| | | | | | | | 100129772 \| |
| 6 | rs9261860 | 30492482 | A | 4.17E−04 | 0.5742 | −3.529 | HLA-E \| MICC \| HCG18 |
| | | | | | | | \| 646520 \| 100129192 \| |
| | | | | | | | 100129772 \| |
| 6 | rs9261862 | 30492717 | G | 2.17E−04 | 0.5636 | −3.698 | HLA-E \| MICC \| HCG18 |
| | | | | | | | \| 646520 \| 100129192 \| |
| | | | | | | | 100129772 \| |
| 6 | rs9261871 | 30493873 | G | 2.27E−04 | 0.5646 | −3.687 | HLA-E \| MICC \| HCG18 |
| | | | | | | | \| 646520 \| 100129192 \| |
| | | | | | | | 100129772 \| |
| 6 | rs9261919 | 30499702 | A | 2.34E−04 | 0.5654 | −3.679 | HLA-E \| MICC \| HCG18 |
| | | | | | | | \| 646520 \| 100129192 \| |
| | | | | | | | 100129772 \| |
| 6 | rs9261923 | 30500139 | A | 2.17E−04 | 0.5636 | −3.698 | HLA-E \| MICC \| HCG18 |
| | | | | | | | \| 646520 \| 100129192 \| |
| | | | | | | | 100129772 \| |
| 6 | rs9261926 | 30500385 | A | 2.47E−04 | 0.5665 | −3.665 | HLA-E \| MICC \| HCG18 |
| | | | | | | | \| 646520 \| 100129192 \| |
| | | | | | | | 100129772 \| |
| 6 | rs9261947 | 30502607 | A | 2.60E−04 | 0.5601 | −3.652 | HLA-E \| MICC \| 646520 \| |
| | | | | | | | 100129192 \| 100129772 \| |
| 6 | rs9501447 | 30505819 | G | 3.36E−04 | 0.5737 | −3.586 | HLA-E \| MICC \| 646520 \| |
| | | | | | | | 100129772 \| |
| 6 | rs1079541 | 30514735 | A | 2.13E−04 | 0.5634 | −3.703 | HLA-E \| MICC \| 646520 \| |
| | | | | | | | 100129772 \| |
| 6 | rs9501467 | 30516949 | A | 4.48E−04 | 0.5935 | −3.51 | HLA-E \| MICC \| 646520 \| |
| | | | | | | | 100129772 \| |
| 6 | rs9295871 | 30519068 | G | 2.91E−04 | 0.5704 | −3.623 | HLA-E \| MICC \| 646520 \| |
| | | | | | | | 100129772 \| |
| 6 | rs9295873 | 30522214 | G | 2.18E−04 | 0.5638 | −3.698 | HLA-E \| MICC \| 646520 \| |
| | | | | | | | 100129772 \| |
| 6 | rs9918306 | 30527750 | A | 2.30E−04 | 0.5732 | −3.683 | HLA-E \| MICC \| 646520 \| |
| | | | | | | | 100129772 \| |
| 6 | rs9295886 | 30529376 | A | 3.91E−04 | 0.5751 | −3.546 | HLA-E \| MICC \| 646520 \| |
| | | | | | | | 100129772 \| |
| 6 | rs35407515 | 30531202 | G | 2.13E−04 | 0.5634 | −3.703 | HLA-E \| MICC \| 646520 \| |
| | | | | | | | 100129772 \| |
| 6 | rs34101875 | 30531337 | A | 1.96E−04 | 0.5615 | −3.724 | HLA-E \| MICC \| 646520 \| |
| | | | | | | | 100129772 \| |
| 6 | rs33986393 | 30532053 | G | 2.16E−04 | 0.5637 | −3.7 | HLA-E \| MICC \| 646520 \| |
| | | | | | | | 100129772 \| |
| 6 | rs9501336 | 30535489 | A | 2.17E−04 | 0.5639 | −3.698 | HLA-E \| PRR3 \| MICC \| |
| | | | | | | | 646520 \| 100129772 \| |
| 6 | rs11966619 | 30537012 | C | 2.49E−04 | 0.5668 | −3.663 | HLA-E \| PRR3 \| MICC \| |
| | | | | | | | 646520 \| 100129772 \| |
| 6 | rs35792611 | 30538854 | C | 6.53E−04 | 0.5875 | −3.409 | HLA-E \| PRR3 \| MICC \| |
| | | | | | | | 646520 \| 100129772 \| |
| 6 | rs3132585 | 30795593 | G | 2.04E−04 | 2.972 | 3.715 | DHX16 \| MDC1 \| FLOT1 |
| | | | | | | | \| NRM \| KIAA1949 \| |
| | | | | | | | TUBB \| |
| 6 | rs3132583 | 30796554 | C | 3.00E−04 | 2.915 | 3.616 | DHX16 \| MDC1 \| FLOT1 |
| | | | | | | | \| NRM \| KIAA1949 \| |
| | | | | | | | TUBB \| |
| 6 | rs2230365 | 31633427 | A | 1.54E−04 | 1.575 | 3.785 | AIF1 \| ATP6V1G2 \| LTA |
| | | | | | | | \| NFKBIL1 \| TNF \| BAT2 |
| | | | | | | | \| BAT1 \| LST1 \| APOM \| |
| | | | | | | | SNORA38 \| SNORD84 \| |
| | | | | | | | SNORD117 \| |
| 6 | rs2229092 | 31648736 | C | 7.30E−04 | 1.882 | 3.378 | AIF1 \| ATP6V1G2 \| |
| | | | | | | | CSNK2B \| LTA \| LTB \| |
| | | | | | | | TNF \| BAT2 \| BAT1 \| |
| | | | | | | | LST1 \| APOM \| LY6G5B |
| | | | | | | | \| SNORA38 \| SNORD84 \| |
| | | | | | | | SNORD117 \| |
| 6 | rs537160 | 32024379 | A | 1.55E−06 | 1.943 | 4.805 | CFB \| C2 \| C4A \| C4B \| |
| | | | | | | | NEU1 \| SKIV2L \| RDBP \| |
| | | | | | | | STK19 \| EHMT2 \| |
| | | | | | | | SLC44A4 \| ZBTB12 \| |
| 6 | rs2072633 | 32027557 | A | 3.04E−04 | 1.513 | 3.612 | CFB \| C2 \| C4A \| C4B \| |
| | | | | | | | NEU1 \| SKIV2L \| RDBP \| |
| | | | | | | | STK19 \| EHMT2 \| |
| | | | | | | | SLC44A4 \| ZBTB12 \| |
| 6 | rs437179 | 32036993 | A | 3.37E−09 | 2.336 | 5.912 | CFB \| C4A \| C4B \| |
| | | | | | | | DOM3Z \| NEU1 \| |
| | | | | | | | SKIV2L \| RDBP \| STK19 |
| | | | | | | | \| EHMT2 \| SLC44A4 \| |
| | | | | | | | ZBTB12 \| |
| 6 | rs386480 | 32054816 | G | 2.52E−09 | 2.355 | 5.96 | C4A \| C4B \| DOM3Z \| |
| | | | | | | | SKIV2L \| RDBP \| STK19 |
| | | | | | | | \| EHMT2 \| ZBTB12 \| |
| 6 | rs389883 | 32055439 | C | 2.66E−09 | 2.359 | 5.951 | C4A \| C4B \| DOM3Z \| |
| | | | | | | | SKIV2L \| RDBP \| STK19 |
| | | | | | | | \| EHMT2 \| ZBTB12 \| |
| 6 | rs2856448 | 32122553 | A | 1.87E−04 | 1.515 | 3.736 | DOM3Z \| RDBP \| |
| 6 | rs185819 | 32158045 | A | 2.73E−04 | 1.498 | 3.64 | RNF5 \| PPT2 \| EGFL8 \| |
| | | | | | | | 653033 \| |
| 6 | rs17207986 | 32187545 | G | 1.36E−16 | 3.953 | 8.268 | ATF6B \| RNF5 \| PPT2 \| |
| | | | | | | | EGFL8 \| 653033 \| |
| 6 | rs1053924 | 32228693 | A | 9.32E−04 | 1.45 | 3.31 | ATF6B \| RNF5 \| PPT2 \| |
| | | | | | | | FKBPL \| PRRT1 \| |
| | | | | | | | EGFL8 \| 653033 \| |
| | | | | | | | 100130536 \| |
| 6 | rs2269425 | 32231617 | A | 4.85E−04 | 1.649 | 3.489 | ATF6B \| RNF5 \| PPT2 \| |
| | | | | | | | FKBPL \| PRRT1 \| |
| | | | | | | | EGFL8 \| 401252 \| 653033 |
| | | | | | | | \| 100130536 \| |
| 6 | rs2269423 | 32253685 | A | 1.68E−04 | 1.492 | 3.762 | AGER \| ATF6B \| PBX2 \| |
| | | | | | | | RNF5 \| AGPAT1 \| |
| | | | | | | | FKBPL \| PRRT1 \| |
| | | | | | | | EGFL8 \| 401252 \| 653033 |
| | | | | | | | \| 100130536 \| |
| 6 | rs443198 | 32298384 | G | 5.76E−04 | 0.689 | −3.443 | AGER \| ATF6B \| |
| | | | | | | | NOTCH4 \| PBX2 \| |
| | | | | | | | AGPAT1 \| GPSM3 \| |
| | | | | | | | FKBPL \| PRRT1 \| |
| | | | | | | | 401252 \| 100130536 \| |
| 6 | rs2894252 | 32453421 | A | 2.67E−04 | 0.6137 | −3.646 | HLA-DRA \| C6orf10 \| |
| | | | | | | | 646668 \| |
| 6 | rs2894253 | 32453518 | C | 1.45E−05 | 1.965 | 4.337 | HLA-DRA \| C6orf10 \| |
| | | | | | | | 646668 \| |
| 6 | rs9405094 | 32454386 | A | 2.66E−04 | 0.6136 | −3.647 | HLA-DRA \| C6orf10 \| |
| | | | | | | | 646668 \| |
| 6 | rs2395157 | 32456123 | G | 2.65E−04 | 0.6136 | −3.648 | HLA-DRA \| C6orf10 \| |
| | | | | | | | 646668 \| |
| 6 | rs9268454 | 32457689 | G | 2.67E−04 | 0.6137 | −3.645 | HLA-DRA \| C6orf10 \| |
| | | | | | | | 646668 \| |
| 6 | rs9268456 | 32457924 | A | 2.92E−04 | 0.6155 | −3.622 | HLA-DRA \| C6orf10 \| |
| | | | | | | | 646668 \| |
| 6 | rs9268461 | 32459879 | A | 2.66E−04 | 0.6136 | −3.647 | HLA-DRA \| C6orf10 \| |
| | | | | | | | 646668 \| |
| 6 | rs17423649 | 32465111 | A | 3.07E−04 | 1.695 | 3.609 | HLA-DRA \| C6orf10 \| |
| | | | | | | | BTNL2 \| 646668 \| |
| 6 | rs12529049 | 32465693 | A | 3.15E−04 | 1.693 | 3.603 | HLA-DRA \| C6orf10 \| |
| | | | | | | | BTNL2 \| 646668 \| |
| 6 | rs16870123 | 32467438 | A | 3.37E−04 | 1.687 | 3.585 | HLA-DRA \| C6orf10 \| |
| | | | | | | | BTNL2 \| 646668 \| |
| 6 | rs2076524 | 32478662 | G | 2.64E−04 | 0.6135 | −3.648 | HLA-DRA \| C6orf10 \| |
| | | | | | | | BTNL2 \| 646668 \| |
| 6 | rs2076522 | 32479157 | G | 2.64E−04 | 0.6135 | −3.648 | HLA-DRA \| C6orf10 \| |
| | | | | | | | BTNL2 \| 646668 \| |
| 6 | rs3806156 | 32481676 | A | 6.24E−04 | 0.6757 | −3.421 | HLA-DRA \| C6orf10 \| |
| | | | | | | | BTNL2 \| 646668 \| |
| 6 | rs9268491 | 32482109 | G | 2.64E−04 | 0.6135 | −3.648 | HLA-DRA \| C6orf10 \| |
| | | | | | | | BTNL2 \| 646668 \| |
| 6 | rs2395163 | 32495787 | G | 3.17E−04 | 0.6026 | −3.601 | HLA-DRA \| C6orf10 \| |
| | | | | | | | BTNL2 \| 646668 \| |
| 6 | rs34330585 | 32498681 | G | 3.00E−04 | 1.74 | 3.615 | HLA-DRA \| C6orf10 \| |
| | | | | | | | BTNL2 \| 646668 \| |
| 6 | rs9268905 | 32540055 | G | 5.94E−07 | 0.5461 | −4.993 | C6orf10 \| BTNL2 \| |
| 6 | rs2395185 | 32541145 | A | 1.16E−06 | 0.5558 | −4.863 | C6orf10 \| BTNL2 \| |
| 6 | rs9368726 | 32546520 | G | 7.45E−07 | 0.5503 | −4.949 | C6orf10 \| BTNL2 \| |
| 6 | rs9405108 | 32546626 | A | 5.94E−07 | 0.5462 | −4.993 | C6orf10 \| BTNL2 \| |
| 6 | rs28772724 | 32617335 | A | 1.34E−08 | 0.4848 | −5.681 | HLA-DQA1 \| |
| 6 | rs28530648 | 32635057 | C | 3.55E−05 | 1.763 | 4.135 | HLA-DQA1 \| |
| 6 | rs28366298 | 32668837 | C | 2.72E−08 | 0.4843 | −5.559 | HLA-DQA1 \| |
| 6 | rs35265698 | 32669312 | G | 6.31E−05 | 0.5203 | −4.001 | HLA-DQA1 \| |
| 6 | rs28605404 | 32677665 | G | 1.42E−06 | 1.967 | 4.822 | HLA-DQA1 \| |
| 6 | rs2516049 | 32678378 | G | 9.52E−08 | 0.503 | −5.336 | HLA-DQA1 \| |
| 6 | rs9270856 | 32678817 | A | 1.30E−06 | 1.79 | 4.84 | HLA-DQA1 \| |
| 6 | rs9271100 | 32684456 | A | 1.45E−06 | 1.784 | 4.818 | HLA-DQA1 \| |
| 6 | rs660895 | 32685358 | G | 8.98E−05 | 0.5616 | −3.917 | HLA-DQA1 \| |
| 6 | rs9271170 | 32685867 | A | 1.45E−06 | 1.784 | 4.818 | HLA-DQA1 \| |
| 6 | rs9271488 | 32696978 | A | 3.08E−08 | 0.4876 | −5.537 | HLA-DQA1 \| |
| 6 | rs9272105 | 32707977 | G | 4.38E−05 | 0.6447 | −4.086 | HLA-DQA1 \| |
| 6 | rs9272143 | 32708781 | A | 2.55E−05 | 0.6415 | −4.211 | HLA-DQA1 \| |
| 6 | rs34276369 | 32722015 | A | 6.62E−08 | 0.4968 | −5.401 | HLA-DQA1 \| HLA- |
| | | | | | | | DQA2 \| |
| 6 | rs2647025 | 32743927 | A | 8.19E−06 | 0.5545 | −4.46 | HLA-DQA2 \| HLA- |
| | | | | | | | DQB1 \| 646686 \| |
| 6 | rs2858331 | 32789255 | G | 1.25E−04 | 1.505 | 3.837 | HLA-DQA2 \| HLA- |
| | | | | | | | DQB1 \| 646686 \| |

### Example 11

Utilizing a GWAS approach of a well-characterized UC cohort and a large healthy control group, the inventors confirmed the contribution of the MHC to severe UC at a genome-wide level of significance and observed more than one ‘signal’ from this locus. The inventors also implicated TNFSF15 (TL1A) in UC severity, with potential therapeutic implications. It was confirmed an association between extensive disease and colectomy, and also demonstrated, for the first time, that a family history of UC is associated with the need for surgery. These observations support the concept that genetic variation contributes to the natural history of UC. The regression model of 46 SNPs presented herein discriminates patients at risk of mrUC and explains approximately 50% of the genetic contribution to the risk of surgery in the cohort. When the risk score was divided into four categories, higher risk score categories had a higher percentage of mrUC subjects (p<2.2×10−16) and predicted earlier colectomy.

The predictive power of diagnostic tests can be evaluated by the area under the curve (AUC), an ROC summary index, which evaluates the probability that one's test correctly identifies a diseased subject from a pair of affected and unaffected individuals. A perfect test has an AUC of 1.0, while random chance gives an AUC of 0.5. Screening programs attempting to identify high-risk groups generally have an AUC of ˜0.80 48. The genetic risk score reported herein yielded an AUC of 0.91.

The inventors calculated operating characteristics in an attempt to determine whether a prognostic test based on these genetic data would be clinically useful. The score of 44 and 47 (out of a possible score of 60) can be used to generate a test with a sensitivity and specificity of over 90%, respectively. The fitted model was robust, given the comparable mean sensitivity and specificity following cross-validation. In addition, likelihood ratios can be used with differing pre-test probabilities to calculate relevant post-test probabilities and are therefore much more generalizable. The Cochrane collaboration has suggested that positive likelihood ratios of greater than 10 and negative likelihood ratios of less than 0.1 are likely to make a significant impact on health care. As can be seen from the data presented herein, these ratios are met with a risk score of 47 and 43, respectively. For example, in a newly diagnosed patient with ulcerative colitis, if the pre-test probability of colectomy was approximately 20% (based on epidemiological and clinical data) and the patient had a genetic risk score of 47 (positive likelihood ratio of approximately 10), then utilizing Bayesian principles, this equates to a post-test probability of colectomy of approximately 75%. If patients at high risk for colectomy could be identified early in their course of disease, then this could have significant consequences for clinicians. Clinicians may suggest earlier introduction of more potent medication for the high risk patients and choose to clinically and endoscopically monitor these patients more intensively. Stressing the importance of compliance with therapy and even monitoring compliance in high-risk patients may also be considered by clinicians.

The inventors have confirmed the association with the MHC and disease severity in UC and the data shows that there may be more than one ‘signal’ from this locus. Furthermore, the inventors have also implicated a realistic therapeutic target and known IBD locus, TNFSF15 (TL1A), suggesting that interference with this pathway is important in severe UC. In addition, the inventors have demonstrated the utility of a model based on GWAS data for predicting the need for surgery in UC. These data demonstrate that the effect of these variants cumulatively they may provide adequate discriminatory power for clinical use. These findings allow a more tailored approach to the management of UC patients and also identify additional targets for early therapeutic intervention in more aggressive UC.

### Example 12

Medically refractory UC (mrUC) requiring colectomy for failure to respond to medical therapy occurs in up to 30% UC patients and remains a significant clinical challenge. The inventors have shown genetic associations with mrUC, which allows for the timely identification of patients at risk for surgery and supports early introduction of more intensive therapy. Genetic loci have been identified as contributing to mrUC using immune-specific Immunochip arrays. These genetic associations also identify novel therapeutic targets for the treatment of severe UC.

### Example 13

TABLE 6
Demographic data

| | mrUC | non-mrUC | |
|---|---|---|---|
| FACTORS | (n = 323) | (n = 639) | P-value |
| Gender (F %) | 43% | 49% | NS |
| Median Age of Onset - yrs | 26 (17-37) | 27 (18-39) | NS |
| (IQR) | | | |
| Smoking (%) | 8% | 8% | NS |
| Median Disease Duration - | 47 (23-128) | 109 (47-208) | 9.5 × 10⁻⁹ |
| months (IQR) | | | |
| Extraintestinal Manifestations | 14% | 6% | 6.1 × 10⁻⁵ |
| (%) | | | |
| Extensive Disease (%) | 82% | 64% | 1.3 × 10⁻⁷ |
| Family History of UC (%) | 26% | 18% | 0.006 |
| Family History of IBD (%) | 32% | 24% | 0.006 |

### Example 14

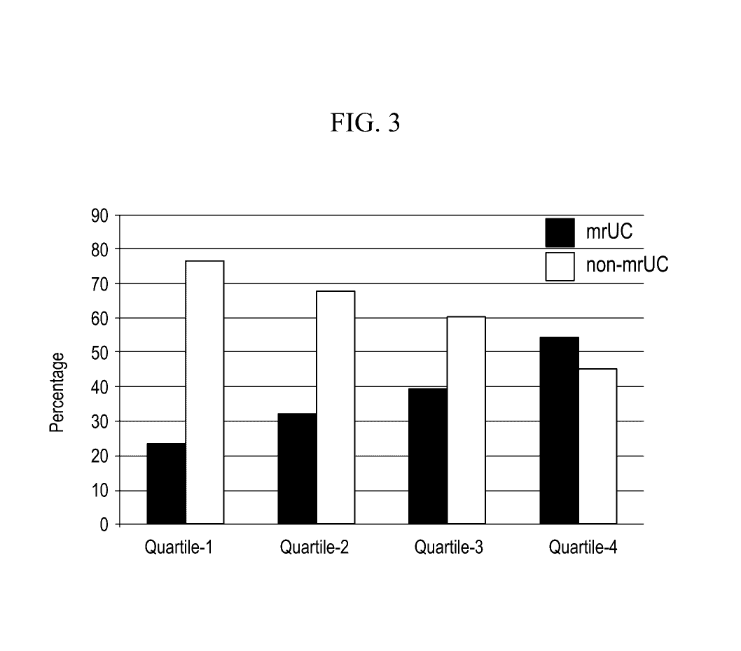

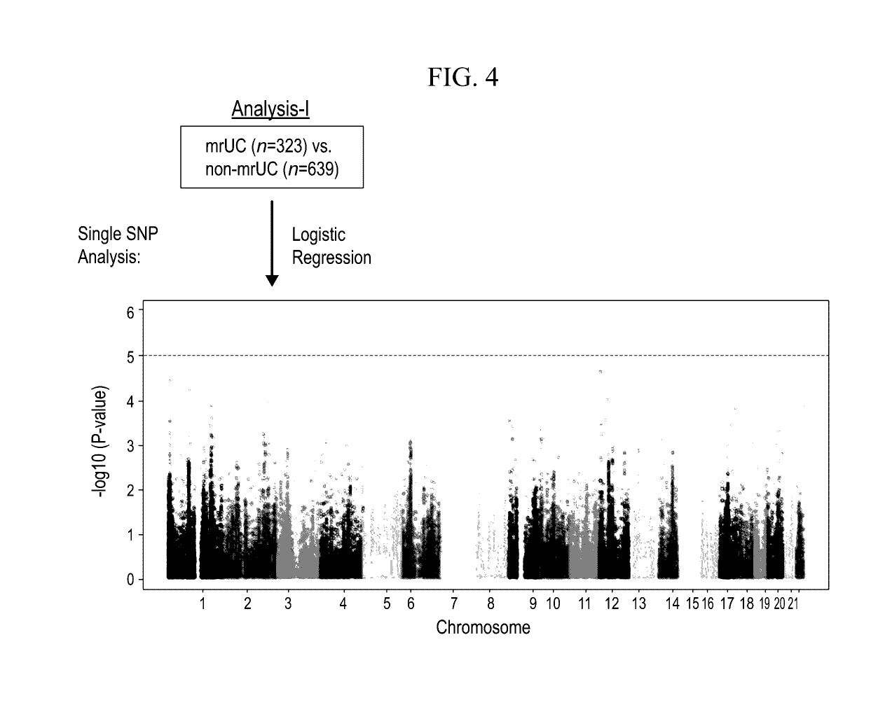

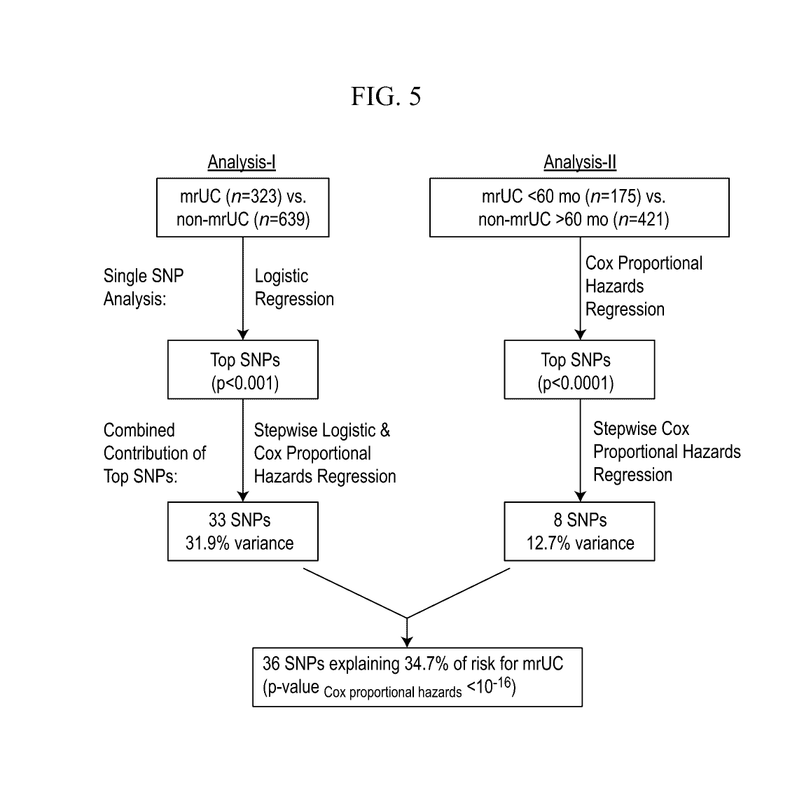

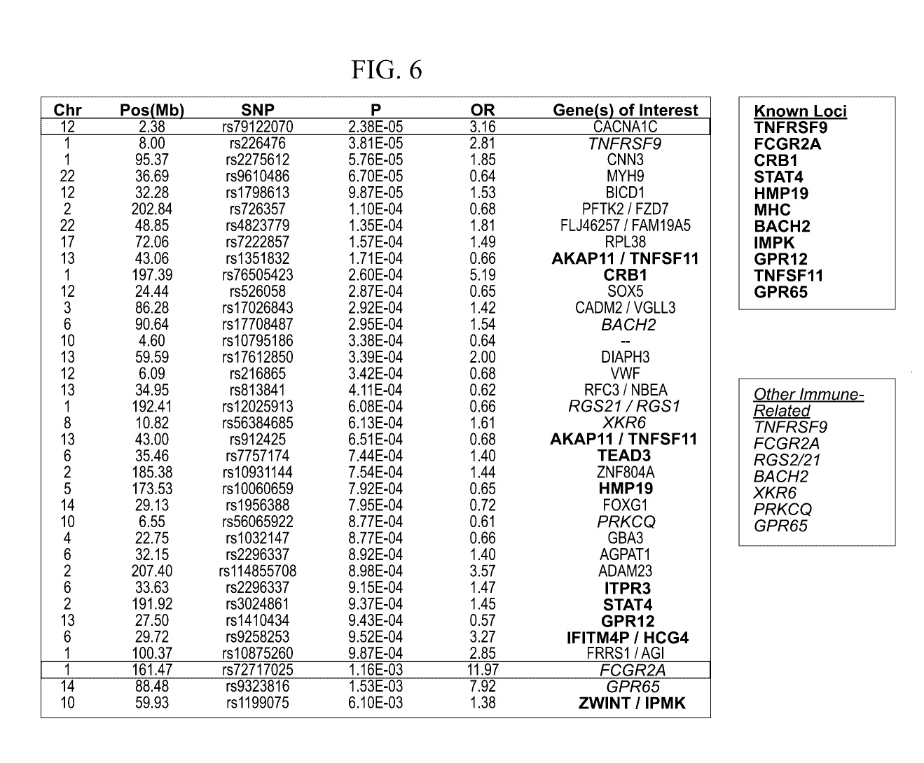

Serological associations with mrUC and Cbir1, ASCA, OmpC and 12 antibody quartile sums calculated within UC, were observed (FIG. 3). The inventors performed a GWAS on 323 mrUC and 639 Non-mrUC subjects. The demographic data of the cohort is summarized herein (Table 6). Following identification of single markers associated with mrUC, the inventors proceeded to a multivariate approach, as performed above to identify the 46 SNPs. The inventors performed a stepwise logistic regression and identified 33 SNPs (Analysis I—Logistic regression: mrUC versus non-mrUC; FIG. 4) and 8 SNPs (Analysis II—Cox proportional hazards regression) that together were associated with mrUC (logistic regression and Cox proportional hazards; analysis schematic see FIG. 5). This analysis resulted in the final risk model of 36 SNPs, which explained 34.7% of risk for colectomy in mrUC (FIG. 6; Table 7).

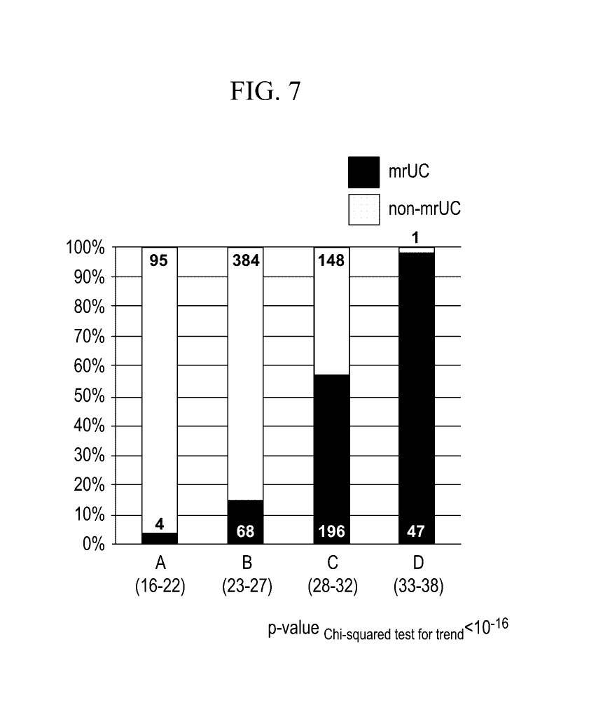

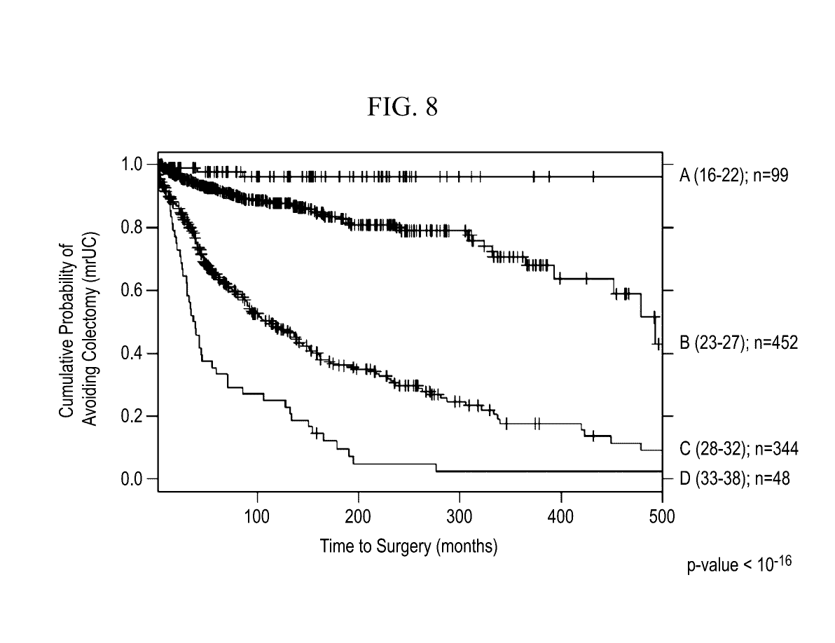

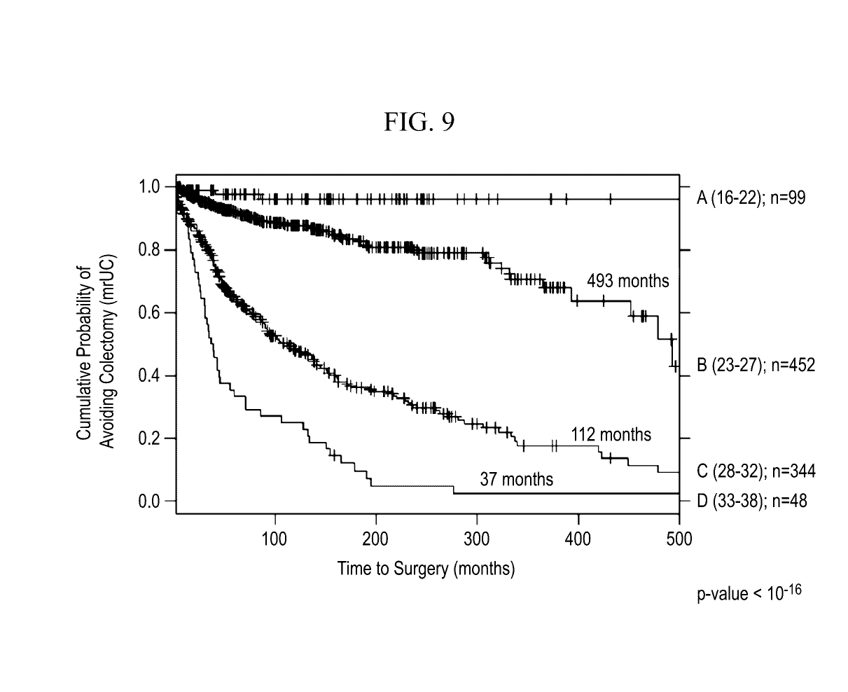

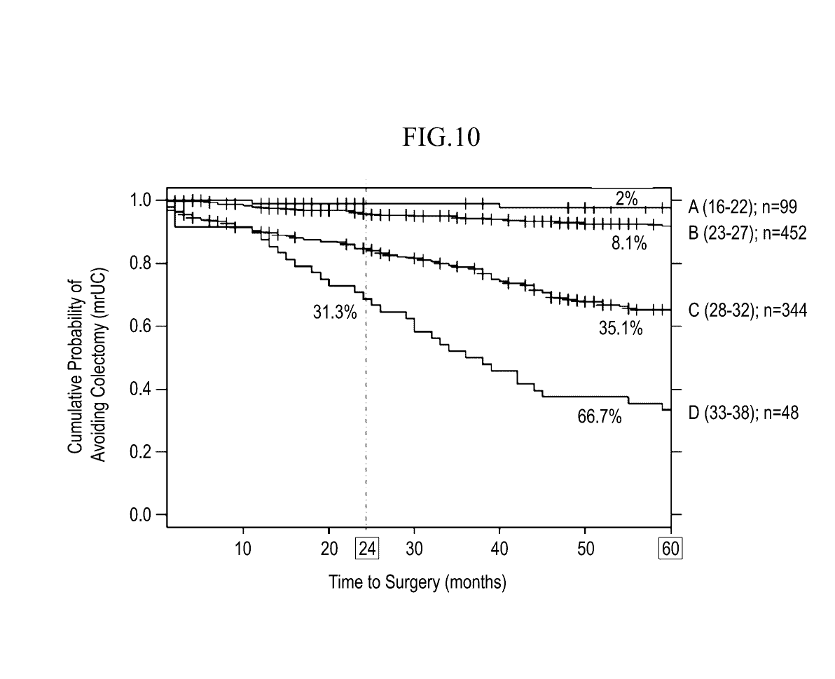

The combination of risk alleles (genetic “burden”) may be useful to identify UC patients at high risk for colectomy. SNPs identified together explain a large proportion of risk: 36 SNPs: 35% risk for colectomy in the mrUC cohort. The inventors calculated a genetic risk score was calculated from the total number of risk alleles (0, 1, or 2) across all 36 risk SNPs (theoretical range: 0-72; observed range: 16-38). Based on the genetic risk scores, the inventors grouped the UC cohort into four risk categories, scores 16-22 (risk-A); scores 23-27 (risk-B); scores 28-32 (risk-C); and scores 33-38 (risk-D). A higher risk score was associated with mrUC, earlier progression to colectomy and shorter overall time to colectomy (FIGS. 7-10). This further supports the paradigm that a group of SNPs, identified by GWAS and combined together may account for a large proportion of the genetic contribution to a complex phenotype to provide a risk score with clinical utility.

TABLE 7
36 SNPs associated with the risk model for mrUC

| Chr | SNP | SEQ ID NO | Gene(s) of Interest |
|---|---|---|---|
| 12 | rs79122070 | 49 | CACNA1C |
| 1 | rs226476 | 50 | TNFRSF9 |
| 1 | rs2275612 | 51 | CNN3 |
| 22 | rs9610486 | 52 | MYH9 |
| 12 | rs1798613 | 53 | BICD1 |
| 2 | rs726357 | 54 | PFTK2\|FZD7 |
| 22 | rs4823779 | 55 | FLJ46257\|FAM19A5 |
| 17 | rs7222857 | 56 | RPL38 |
| 13 | rs1351832 | 57 | AKAP11\|TNFSF11 |
| 1 | rs76505423 | 58 | CRB1 |
| 12 | rs526058 | 59 | SOX5 |
| 3 | rs17026843 | 60 | CADM2\|VGLL3 |
| 6 | rs17708487 | 61 | BACH2 |
| 10 | rs10795186 | 62 | |
| 13 | rs17612850 | 63 | DIAPH3 |
| 12 | rs216865 | 64 | VWF |
| 13 | rs813841 | 65 | RFC3\|NBEA |
| 1 | rs12025913 | 66 | RGS21\|RGS1 |
| 8 | rs56384685 | 67 | XKR6 |
| 13 | rs912425 | 68 | AKAP11\|TNFSF11 |
| 6 | rs7757174 | 69 | TEAD3 |
| 2 | rs10931144 | 70 | ZNF804A |
| 5 | rs10060659 | 71 | HMP19 |
| 14 | rs1956388 | 72 | FOXG1 |
| 10 | rs56065922 | 73 | PRKCQ |
| 4 | rs1032147 | 74 | GBA3 |
| 6 | rs2269423 | 75 | AGPAT1 |
| 2 | rs114855708 | 76 | ADAM23 |
| 6 | rs2296337 | 77 | ITPR3 |
| 2 | rs3024861 | 78 | STAT4 |
| 13 | rs1410434 | 79 | GPR12 |
| 6 | rs9258253 | 80 | IFITM4P\|HCG4 |
| 1 | rs10875260 | 81 | FRRS1\|AGI |
| 1 | rs72717025 | 82 | FCGR2A |
| 14 | rs9323816 | 83 | GPR65 |
| 10 | rs1199075 | 84 | ZWINT\|IPMK |

### Example 15

### mrUC Network Analysis

Analysis of 962 subjects (323 mrUC and 639 non-mrUC) resulted in 6573 candidate SNPs (logistic regression analysis (p<0.05)>1742 genes. A calculated gene-based logistic regression score was used to obtain genes with a maximal AUC>0.56 selected for network construction. The network was constructed using pairwise Pearson correlation coefficient (p<10−7) between gene scores and protein-protein interaction database (STRING). Pathways associated with mrUC networks revealed cytokine-cytokine receptor interactions (p=1.5×10−5), T-cell receptor signaling pathway interactions (p=0.0001) and Rheumatoid arthritis (p=0.0015). This analysis identified relevant pathways for further investigation of potential new therapeutic targets for mrUC.

### Example 16

### Role for MHC in UC Severity

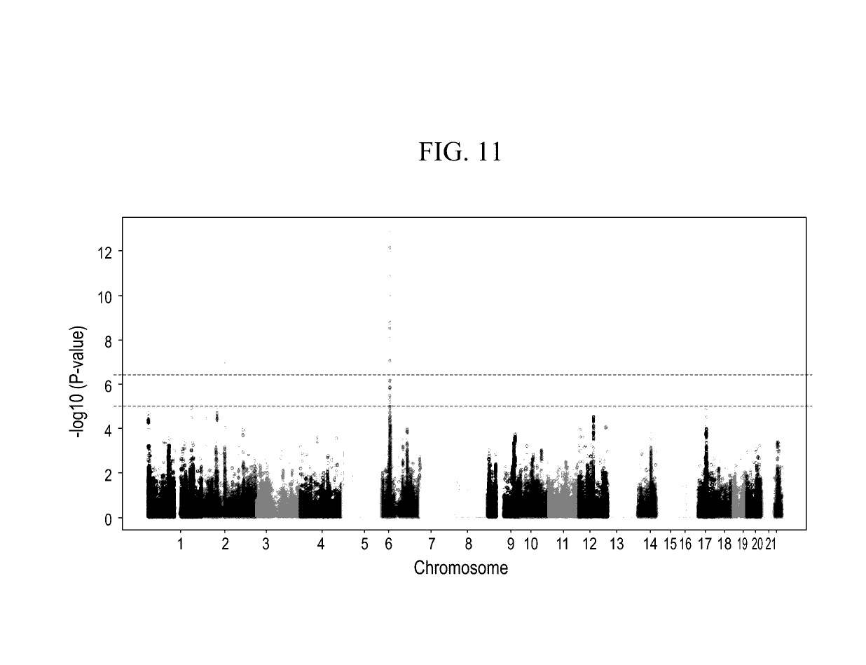

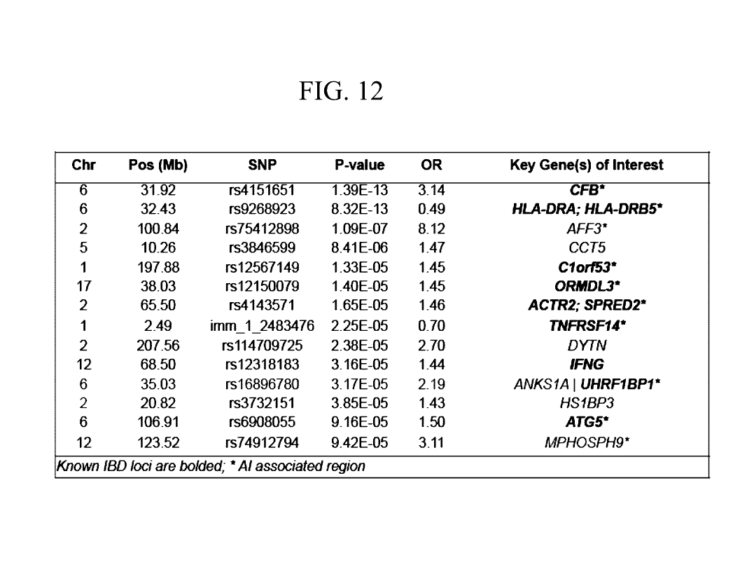

Stringent sample and SNP quality control of 323 Caucasian mrUC subjects and 5190 controls was performed to test single-SNP associations with regression analysis corrected for 4 principal components. Results demonstrated the association of MHC with UC severity (FIG. 11-12; Table 8).

TABLE 8

| Chr | SNP | SEQ ID NO | Gene(s) of Interest |
|---|---|---|---|
| 6 | rs4151651 | 85 | CFB |
| 6 | rs9268923 | 86 | HLA-DRA\|HLA-DRB5 |
| 2 | rs75412898 | 87 | AFF3 |
| 5 | rs3846599 | 88 | CCT5 |
| 1 | rs12567149 | 89 | C1orf53 |
| 17 | rs12150079 | 90 | ORMDL3 |
| 2 | rs4143571 | 91 | ACTR2\|SPRED2 |
| 2 | rs114709725 | 92 | DYTN |
| 12 | rs12318183 | 93 | IFNG |
| 6 | rs16896780 | 94 | ANKS1A\|UHRF1BP1 |
| 2 | rs3732151 | 95 | HS1BP3 |
| 6 | rs6908055 | 96 | ATG5 |
| 12 | rs74912794 | 97 | MPHOSPH9 |
| 1 | rs2281852 | 98 | TNFRSF14 |
| 1 | rs2281852 | 99 | TNFRSF14 |

### Example 17

### Additional Summary and Conclusions

Cross-validation and bootstrapping was performed to validate the fitted logistic regression model. The model was able to identify a dataset for independent replication. A multivariate model will be built by integrating clinical, serological, and genetic associations. A truncated genetic analysis can then identify a patient population at risk for colectomy that would benefit from early intervention and identify therapeutic targets (Table 9), which would address an unmet medical need.

### Example 18

TABLE 9
Potential Therapeutic Targets

| Genes | Pathways |
|---|---|
| ORMDL3, CCT5 | Protein folding & ER stress/UPR |
| MYH9, ADAM23, CADM2 | Cell adhesion & cell-cell interaction |
| TNFRSF14, IFNG, | T-cell mediated immune response |
| TNFRSF9, STAT4, PRKCQ, | |
| TNFSF11, BACH2 | |
| ATG5, PRKCQ | Autophagy |
| TNFRSF9, IL6R, | Cytokine-cytokine receptor interaction |
| TNFRSF18/TNFRSF4, | |
| CCL21, TNFSF15, TNFSF11, | |
| TNFRSF13B, CCL2/CCL7, | |
| TNFRSF6B | |

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method of determining the need for colectomy in a subject with medically refractive UC (mrUC) comprising:
obtaining a sample from the subject;
assaying the sample to detect the presence or absence of mrUC genetic risk variants, wherein the mrUC genetic risk variants are selected from the group consisting of SEQ ID NOs: 1-99;
calculating a genetic risk score based on the detection of the mrUC genetic risk variants; and
determining that the subject has an increased likelihood of needing colectomy if the calculated genetic risk score is at the high end of the observed range and determining that the subject has a decreased likelihood of needing colectomy if the calculated genetic risk score is at the low end of the observed range.

2. The method of claim 1, wherein the genetic risk score is obtained by calculating a total number of risk alleles for all the mrUC genetic risk variants assayed, wherein the risk allele for each mrUC genetic risk variant assayed is 0, 1 or 2.

3. The method of claim 2, further comprising obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected.

4. The method of claim 3, wherein the number of mrUC genetic risk variants assayed is 46, the theoretical range is 0-92 and the observed range is 28-60.

5. The method of claim 3, wherein the number of mrUC genetic risk variants assayed is 36, the theoretical range is 0-72 and the observed range is 16-38.

6. The method of claim 1, further comprising prescribing colectomy to subjects having a genetic risk score at the high end of the observed range.

7. The method of claim 6, wherein time to colectomy is lower in a subject with a genetic risk score at the high end of the observed range and time to colectomy is higher in a subject with a genetic risk score at the low end of the observed range.

8. The method of claim 7, wherein the time to colectomy is 10 to 70 months from detection.

9. A method of diagnosing susceptibility to medically refractive UC (mrUC) in a subject, comprising:
obtaining a sample from the subject;
assaying the sample to detect the presence or absence of mrUC genetic risk variants, wherein the mrUC genetic risk variants are selected from the group consisting of SEQ ID NOs: 1-99;
calculating a genetic risk score based on the detection of the mrUC genetic risk variants; and
diagnosing susceptibility to mrUC based on the calculated risk score, wherein a subject has an increased susceptibility to mrUC if the calculated genetic risk score is at the high end of the observed range and a subject has a decreased susceptibility to mrUC if the calculated genetic risk score is at the low end of the observed range.

10. The method of claim 9, wherein the genetic risk score is obtained by calculating a total number of risk alleles for all the mrUC genetic risk variants assayed, wherein the risk allele for each mrUC genetic risk variant assayed is 0, 1 or 2.

11. The method of claim 10, further comprising obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected.

12. The method of claim 11, wherein an increase in the number of risk alleles detected signifies an increase in susceptibility to mrUC.

13. The method of claim 11, wherein the number of mrUC genetic risk variants assayed is 46, the theoretical range is 0-92 and the observed range is 28-60.

14. The method of claim 11, wherein the number of mrUC genetic risk variants assayed is 36, the theoretical range is 0-72 and the observed range is 16-38.

15. The method of claim 9, further comprising prescribing colectomy to subjects diagnosed with a susceptibility for mrUC and have a genetic risk score at the high end of the observed range.

16. The method of claim 15, wherein the time to colectomy is lower in a subject with a genetic risk score at the high end of the observed range and the time to colectomy is higher in a subject with a genetic risk score at the low end of the observed range.

17. The method of claim 16, wherein the time to colectomy is 10 to 70 months from detection.

18. A method of treating mrUC in a subject, comprising:
obtaining a sample from the subject;
assaying the sample to detect the presence or absence of mrUC genetic risk variants, wherein the mrUC genetic risk variants are selected from the group consisting of SEQ ID NOs: 1-99;
calculating a genetic risk score based on the detection of the mrUC genetic risk variants;
diagnosing susceptibility to mrUC based on the calculated risk score, wherein a subject has an increased susceptibility to mrUC if the calculated genetic risk score is at the high end of the observed range and a subject has a decreased susceptibility to mrUC if the calculated genetic risk score is at the low end of the observed range; and
prescribing colectomy to the subject with an increased susceptibility to mrUC.

19. The method of claim 18, wherein the genetic risk score is obtained by calculating a total number of risk alleles for all the mrUC genetic risk variants assayed, wherein the risk allele for each mrUC genetic risk variant assayed is 0, 1 or 2.

20. The method of claim 19, further comprising obtaining a theoretical range and an observed range based on the genetic risk score, wherein the theoretical range consists of the minimum and maximum number of risk alleles possible based on the number of mrUC genetic risk variants assayed and wherein the observed range consists of the actual minimum and maximum number of risk alleles detected.

21. The method of claim 20, wherein an increase in the number of risk alleles detected signifies an increase in susceptibility to mrUC.

22. The method of claim 20, wherein the number of mrUC genetic risk variants assayed is 46, the theoretical range is 0-92 and the observed range is 28-60.

23. The method of claim 20, wherein the number of mrUC genetic risk variants assayed is 36, the theoretical range is 0-72 and the observed range is 16-38.

24. The method of claim 18, wherein the treatment is colectomy and is prescribed to subjects diagnosed with a susceptibility for mrUC and have a genetic risk score at the high end of the observed range.

25. The method of claim 24, wherein the time to colectomy is lower in a subject with a genetic risk score at the high end of the observed range and the time to colectomy is higher in a subject with a genetic risk score at the low end of the observed range.

26. The method of claim 25, wherein the time to colectomy is 10 to 70 months from detection.

27. A kit for prognostic use, comprising:
a single prognostic panel comprising one or more medically refractive ulcerative colitis (mrUC) genetic risk variants comprising SEQ ID NOs: 1-99.

28.-34. (canceled)


SEQUENCE LISTING (99 sequences)

SEQ ID NO: 1 - DNA, 844 bp, Homo sapiens
ctggcagagcttaggtctcctgtaagcgcgtgcctgctggctgccccgatccctgcagtgatcactgcagaagaacagagagggaggaagtgcctggtcagacagcagcagacctgagtggttgagaacaggaaggggggcaggcaggagagaagaatctaggggttctcgactccaggccctgcaggggtgggtacgtttccataactggactggtttcttgatctcacccaattctttgaagagatctttttttgacataagtggtgtgggagagttcttgttgcttgccattagccacctctggctcagatgagaaaggaaaattgggtgggtggggtcagctacctgactgatctccgaggacagcacccagttgtccttggccacaagcatcttcaaggaggagacgttattgtagctcaggtacacctgaaagagtacacaggtgctaccactaccccrcccccttccttcagggcccctgagccaaggccaagggtgtgccagagaggaacctgggccaggaatgggttgggcggtgggtgggtggtgcggcaggtgctgtggaaagagtcctgggcgttgagaagggaggtccaggcttgggcttggctagacccactgtgtgaacctagacaagctactctgcctctccaggtctccgcttcacctaccaaacagggacgcaggccagctctttgctaatggtagcaatcatgaggcgtgtcattatatcctgctctccccgggcttcttcaaacccgttctgacaagcagggaaggttggggcccggtgggtctgaagactgtctttcgagcagagagccttacctggtgctag

SEQ ID NO: 2 - DNA, 601 bp, Homo sapiens
cccagtactatagtacttctatctattcttaggagaggcttaatattttgcatggtgagaattaatcatattgaataagtaattgaataagcaacaccacattacttacctgcagaccaattacactttggccagcttttaattttccttcatcaaaacgtcttgtttgtttttctgcatacttaactccaatgtcaatggttgtatggaatccttttgttttagcctagacagaaacatgcacactaactgaaaaggccaacagagtttcacagaaggaacaacaaagaaatgatatgtycatagacaaaggggcccaaactagagataaaaagtatttaaggctgttcaataatatactgcatcatttgaaatgtgagtgttctaagtaagctatctagtacgatgcagctgatgtcagcagcctttccacaaagctactaattacagaagagacaggccccgccccacctggtctgaacccccatgaaaaagcaatgatgcaattcatcaccaatgtggtgggaatctgttaaccagccattaaagacatgtacgtaccagacctgctagagccaccagagtagtctgaacctgggtc

SEQ ID NO: 3 - DNA, 601 bp, Homo sapiens
tcaggatacctccatagagtcccaaaatgagcacaaagttgcaaccagtggccacaccaatcactgtgccatgggccctagcatgccagcttgtgtccacacatgccaaccgcattagtggtgccaagtcacaaaagtaatgggccacctctttcaagcagaagggcagagtggcagtgagggtggctggcacaagtgcagctgagaagccagccacccaactggccccagctagtcgtaactgtacctgtctgctcatgagtgcgtggtagtggagtgggtggcagatgataaggtagckatccagtgccatgacacccagcaggtagcactcagtcatccctaaggaatgaaagacatagctgaataaagcatacagctgatgagacgggtgaacacccttggagcaaggtgtgcagcagcatgggcactgtggtgctgacataccacacctctacaaaggagaggacactgataaataagtacatgggcgtgaacagtccagaatctaactgtaccaggacaatgatgagaatgttccctgcaagtgtgaggagatagatgcatagggtccccaagaaggcaagaggttgtagggtcc

SEQ ID NO: 4 - DNA, 9326 bp, Homo sapiens
  misc_feature (4505)..(4604): n is a, c, g, or t
taacttttatttttacctcttaagtatcttaaaataatagtggaagtagtattttaactaggctttacatatagattataaattttaaattaagtactaaaatcaaatacatagactttcagtttacagtctgggatataagaagctgggaaggtgctactccatcctaacaccaggtaaaaagctgacatacttaaaaaaatcaatgacacttggccaggcgcggtggctcacgcctgtaatcccagcactttgggaggacgagacgggcggatcacgaggtcaggagatcgagaccatcctggctaacacggtgaaaccccatctctactaaaaatacaaaaaattagccgggcgtagtggagggtgactgtagtcccagctactcgggaggctgaggcagaagaatggcgtgaacccggaaggcggagcttgcagtgagctgagatcgtgccactacactcttgcctgggcgacagagcgagactctgtctcaaaaaaaaaaaaaaaaaaaaaagtcaacgacacttcttagatctctaagaaaattgagggccaaggcaaactgttcacaaaattggaaagaatgaaaggcaagtatgtagaatcacaatttactggagcagaaacccatgagcaaaaacctctaggaaccagtgcttgggtagggaaaactgaactgtaatatatgtggtaggtgcattaagcatttactctacgggacccagttaggaggcttttttgaaaagcatactttgtgagttttacttccaggggcttaatctggttcttagtgaatactgcagaaatactccctcttgcttcctgcaaggggaaggaaaactaacgattttgaattaagatggagcattctgttcttagcaaggcctgccctcaggaaaaactatttaaccagaagctaacatgctagggttttatcagaggcttactgacctgggggaagagaaaaacccaaccccagcccactctagccaccccgtaccacataagaggaggaaaaactgaggtgcatttgtgaagttcatagtccagaggctcaggctgacacagttccttttacatagtacatcatgtttggcaattaagaaaaaattacaaggtacgccaggaggcaaaaaaaaacatagtttgaagagacaaagcaagcataaaaaccagccttagatatggcagggatgttgaaatttgaaagaactatgagtaataagctaaggcctctaatggataaaatagcatgcaagaacagatgggcaatgcaagcaaagagatgaaaatttctcgagcttgaggatatattatttatttttcttttttttcttccacaacagttatctcagaagaggatatattaataggaacttccaaaactaaaaaacaaagagaacaaagactgaaaaaatcagcacagaatgtccaaggattgtgggacaactacaaaaggtataacttatgcattaatggaaatacaaggagaggaaagagagaaaaaaaggaagaactatctgaaatataatgaatgagaatttccccaatttaatgtcaaataccaaagcaaagatctaagaatctcagagaataccaagcagaagaaacaccacaaaaacctctagatttacacatatcatattcaaactacagaaaatccaagataaagaaaaattctggtccagctatggtggctcacacctctaatcccagcactttgggaggctaaggcgggcagactgcttgagcctagcagctcgagaccagcctggacaacatggcaaaaccccatctctacatactaaaaaaaaggaaaaaaaaatcctgaaagaaaacactttaccgatagaggagcaaagataagaacttcatctgacttcttcttagaaaccaggcaagcaagaggagactggagtgaaatatcttgcgttgagaaaaaaaatctggcaatctaaacctctgcaccctatgaaattatcattcaaaagtaaaacaggactgggcatcatagctcacgcctgtaatctcagcactttggaaggccaagagggaggactgcttaagcccaggagttgtgcagcagcctgagaaacaccgccagaccttatctctacaataataaataaaataaatatatatatatatatatttttttgagacagagtctcgctctgtcacccaggctggagtgcagtggtgcgatcttggctcactgcaagctccgcctcctgggttcacaacagtctcctgcctcagcctcccgagtagctgggactacaggcgcccaccaccatgcccagctaattttttgtatttttagtagagacagggtttcaccatgttagtcaggatggtctggatctcctgacctcgtgatctgcccacctggcctcccaaagtgctgggattacaggcatgagccaccgcacccggccaataaaataaaattttttttaagtgaaagaaaaatactttctcagagaaaaaaacttgagaaaatctgtcgccagtaggactgccttgtaagaaatgatttttttctttttttctgagacggaatcttgctctgttgcccaggctggagtgcaatggagcgattttggctccctgcagcctccacctcccaggtccaagcgattctcgtgcctcagcctcccaagtagctgggactataaatgccaccaggcccggctaatttttgtatttttagtagagacggggtttcaccatgttggccaggctggtctcgaactcctgacctcaagtgatctacccacctcagcctcccaaagtgctaggatcacaggcgtgagccaccacacctggccagaaatgttaaatgatgttctttaaagaaaaagaaaattttatatgtcagaaactcagatataaataaaggaaatgtatcaaagaaagaatacgttgaaggtaaaatacaaacttttattttccttattcctaattgttccaacagataacagtttgttcaaaataataacagcaacagcatatttgattatatatgattatgtgtatatatgcttatgcatgcttaggtataaataaaataatgagagcaatgatacaaaggaaaggagggatgaattaggattattttgttattatcaggtactagcactacccatgaagctgtacagtgttatttgaaagtggatatggattagttgtaaaagtatataatgtaaactctagggcaaccattaaagaaagctaaaagaaaaaaaagtataacctatatgctaagaaagcagagaaaaatggaattatataaagtaaaccataaaacacaaaaaaagagtggaatataaaaatagaaataaagaatatgagcaacaaatagaacatagtaataaacacggtagatatttattactatatgataatmcaactacatcaacaaccactttgaaatctactttaaacataaagacacagatagattaaaagtaaatggatgaaggccaggtgtggtggctcatgcctataatcccagtactttgggaggccgaggtgggtcccatcgcttgagccaaggagttcaagaccagcctggggtacactgacaccccatgtctatacaaaaaatacaaaaattagcaggtgtggtgtcttgtgcctgtagtcccagctacttgggaggctgaggtgggaggattgcttaagtccaggaggttgaggctgtagtgagccaccgcactccagcctaggctacagagggagaccctgttctcaaaaacaaacaaacaaacaaacaaacaaacaaacaaacaaaacagggctagtctcagtggctgacacttgtaattccagcactttgggaggctgaggcaggcagatttcttgtggtcaggagttccagaccagcctggccaacatggtgaaaacctgtctctactaaaaatactaatattagccagatgtggtggaggacgcatgtaatcctagctacttgggaggctgaggcatgagaatcatttgaacccaggaggcagaggctgcctgggtgacagagcaagactttgtctcaaaaaaaaaaaaaaaaagtaaatggataaagaatatgccatgctaacactaatcaaaagaaagcaggagcagttatatcaatttcagacaaagctgactccagagcaagaaaaggtgtcaggaataaaaaggggcattatggctgggtgtggtggctcacacctgtaatcccagcactttgggaggctgatgcgagcggatcacaaggtcaggagatcgagactgtcctggctaacacggtgaaaccccgtctctactaaaaatacaaaaaaattagccgggcatggtggcgggcacctgtagtcccagctactcaggagactgaggcaggagaatggcatgaacctgggatgtggagcttgcagtgagccgagatcacgccactgcactccagccaagggtgacagagcaagactctgtctcaacaaaaaaaaaaaaaaaaaaaaaaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnataatgagaaaggggtcaatgttccaagaagacataataattttttttttttttttgagacagagtcttgctctgtcacccaggccagagggcagtgttgcaatgtcagctcacgcctcctgggttcaagcaattctcctgtctcagcctcccaagtagctgggattacaggtgtctgccaccacacctagctaattttgtatttttagtagagacggggtttcaccacattggccaggctggtctcgaattcctgacctcaagtgatctgcccgcctaagcttcccaaagggctgggattacaggggtgagccaccacacccggccaacaatacttaatatttatatgcctaataacagaacatcaaaacatgaggcaaaatgattaactattgacaaatagatcaatccaccattatagttggagattttgatacccctctatcagaaatgaacagattcaatgggcagaaaatcagcaaggacataaactcaacaataccatcaattaactggatataattggtatctatagactacttcatccaacaaccatagaatatactttctactcaaggtcgtgcggaaaattcatgaagagctcattctgggccacaaaacataccttaacaaaattaaaacaatagaaatcatacaatgtatgctcttacaccacaatgaacttctactagaaatcaataacaaagaaagctgtaaaatccgaagatatgtctaaataatacgagtcaaaaaagcaatctaaaagaagaaatgaaaaaatattctgaactagataaaaataaaaagacaacttgtcaaaatttgtgtgatgcagccaaagcagtgcttagtgggaaacttgtagtatcgaatgcatatattagtaaagaagacttaaaatcagtaatttaagcttccaccttaggaaactaaaaaaaaaaaataggaaattaaatacaaagtaatcagaataatataaactagagcagaaatcaataaaattgaaaacagaaaatagaaaaaaatcaacaaaaccaaaagctggttgtttaaaaagatcaatgatactgataagcctctagctaggctaagaaaaaagagagaggacacaaattattaatttcagaaatgaaaaaggggcatcactacagatcctatggactttaaaaagataaactccttgaaatttctaaatttgcacaagaaaaaaatggaaggctctgaataggcttatatctactaaataaattgagtcaataattaaccttccaaaacagatagcaccaggcccagatgggttcactaaggaattctactaaacatttgaggaagaaattatacccaacctctgttatgttttagaggatagaagcagaggaaatacttcctaactcttctccaaggttagcattaccttaataccaaaatctgacaaagacacagtgaaagaaaactataagccaggcacaatggaatgtactcatagtcctagctactgggaggctgaagtgggaggatcacttgaacccaggagtctgcgcctagcctgggcaacatagatcccctgtccaaacatgaaaagaaaaaaactatagatcagtatctttcataaacacagatgcaaaaatcctcaataaaatattagcaaattgaatccaatgatgtataaaagaatttaagatcacggccaaatgagttttttttttttttttttttttttttttttttttgagacagtgtcttgctctgtcacccaggccggagtgcagtggcacgaactcggctcactgcaagctccgcctcccaggttcacgccagtctcctgtctcagcctcccgagtaactgggactacaggcactggccaccacgcctggctaatttttttgtatttttagtagagacggggtttcaccatgttagccaggatggtctcgatctcctgacctcgtgatctgcctgcctcggcctcccaaagtgctgggattacaggcgtgagccaccgcacccggccccaagtgagatttacctgaagtatgcaaggctgattcaatgtttgaaaattaattaatataatccatcacatcagtaggctaaagaagaaaaatatgatcagattgatagaggcaaagtatttgacaaaattcaacactcattcatgataaaaactctcagtaagctaggaatagacaacgtcctcaacctgataaaaaaaaaaaaaatctacaaaaaaacctcatgtctaatatcatacttaatggtgagaaactagaagttttcccactaagatcaggaagaaggcaacaatgtcctctcttatcactcttttcaacatcatactggaagtcctagctaatgcaataagataagaaagagaaattaaaggtagaattgggaaggaaaaaaacaactatctgttttaggcgggtgacatgactgtctacgtagacaatctgaaagaaatgagaaaaaaaaaaaatccctggaactaataagtgactacagcaaagttgcaggatacaaagttaacatacaaaagtcaattgctttgtcaaacaataaacaattatgatgtgaaattaaaaacacagtatcatttaccttaacaccccccaaaatgaaatacttaagtataaatccaacaaatatgtacaagacctatgtgagcaaaactctgatgaaagaaatcaaagaactaaataaataagagatatttcatgttcatggataagaagacacaacactgtcaagataccagttcttctcaacttgatctataaattcaattcaatacaagtcaaaatcccagcgagttgttttatggatattgacaaaatgattctaaagtctatattgaaaggcaaaagacctactatagccaacacaatattgaagtagaagaacaaagttgaaggactgacactattcaacttcaaaacatactataaacctacagtaatcaagacagtgtggtagtggcaaagaataaaataaaatagatcaatggaacagaatagagagcccagaaataaatccacatagtcaattatcttcaacaaatgggcaaaggcagttgcgcaacaaatgggcaacagtggagaaaagtcaagtcttcaacaaatggtgctgcaacaactggacatccacaaacaaacaacaaaaaaatgaatctagacaaaaaccttacacctttcacaaagggaatcacaaacatagatgtaaaatccaaaactataaaactcctagaaagtaacacaggaaaaaaaatctagatgactttggttttgtgacaaccaactttagatatgacatcaagggcatgatccatgtaagaatgaaatgatgagctgaacttcattaaaattaaaaacttctgtttggttaaagacattgtaaagagaatgaaaagacaagccacagactgggagaaaatatttgtaaaatatacatccgataaggaactgctttccaaaatatacaaagaactcttaaaacccaacaataagaaaacaaacaattggattaaaaaatgagccaaagaccttaacagacacctcaccaaagaagatatacagatggcagataagcatatgaaaagatggtcaatgaggaaatgtatatgtcatcagggaaatgcaaatttaaataataagataccacaacaaacctattagaatggccaaaatccagaacactgacaacaccaaatgctaacgaggatgtggagcaacaggaaatcccattcactgctgctgggaatgcaaaatggtacagccactttagaacacagcttgtcagtttcttatataactaagcatactcttaccatatgaaccagcaattacactccttggtatttacccaaaggagctgaaaacttacggccacacaaaaacctgcacacagatgtttatggcacctttattcataattgccaaaatttgaaagcaaccaagatgtctttcagtttgtcaatgagtaaataaactgtggtacatcccgacaatgtaatattactcaatgctaaaaagaaatgagctatcaagccacgaatagacatgaaagaaacttaaatgtatactagtaagtgaaagaaaccaatataaaaaggctatatactgtatgacttcaaatatatgacagtctgcaaaagataaaactatggaaacagtaaaaagatcagtgatttccaggggctcaggggaagagataaaaaggtgaaacacagaagatttttagggcagcaaaactactctatatgatacataatggtggatacatgatattatacatttgtcaaaactcttaaaatgttccacatctttaaatacattaatgtgaactatggtctttaggtgaaatgatgtgttaatgaaagttcactgactgtaacaaatgtaccatctgctgggagataatagggggagactacacatgtgtggggtcaggaagtatatgggaaatatctgtatcttcttctcaattttgctatgaacctaaaattggtctaaaaaaataaagtgtattgaattaaaaatcaaatataacaaggatcgataaaaatatcacatagtgatatttagacatagtgtagtagtacttagcttctggctccctttgcttattgatctctctcaaactctctcacacacacaacacctgttcaaatataacatattagctttgtttttacttctactatttaaaagaaaaaattaaaaaca

SEQ ID NO: 5 - DNA, 537 bp, Homo sapiens
atcagtctcaaatctaaaccaattttggaggtgaggggtggtccatcttgcacctataaaagtctcattactggtggtgttaacttttcttggtcaagacagtgtctgccaggcttctacaactagaaagtcatgtttttccccttttgtaatcaatagatattttctggagagataatttgagactatgtaaaatttcatttaaaccctactttcacccaccagtttgggtgtccattaatgttaattattactgtgatagttgtcaaatggagaccttctgatatcatcaatccttctacatttattagttggctttctactgtaagggagacttctcttgccttcccatttgtttattcayttattcatttatttcttttagcagtgtggagccacatatttgtgttttattcagtagattagaatctgtaactgtcattatttatcttgatgctcaaatcaccagatgtggccaatgaaagcctctttaaactggcttctgagaatttttgttatctcctcattattgtttga

SEQ ID NO: 6 - DNA, 501 bp, Homo sapiens
atatactagaacaaaaggaatgaatcagggctctcacaagcaaatgagaatataagatcatccaacctctgaacaccaagaaattataattgtaacacaatcatgtataacgtcagtggtgaattccctttctgctccctatattacaagttgtttccctctctctcccattcatcctatctctctactcaacttcacctttattctggcagacaaagtacacacgcatggattgctatgtagaccattcrcaagtaagaatgaaaaccactacatctggtaaagacccatctggccacgtgaaatcagcaaacagtaatttctctctcaccactggaatagttgctataagacggactgcaagagaggactagaagaacatgtgaagaagagtgagaccaccacgctggtcctctacttgatgatgaacggattgtggggaggcaactgagggaatctcccttgatcttttgggaaatatgccctcatcaagactgaaaa

SEQ ID NO: 7 - DNA, 653 bp, Homo sapiens
tttatcacagcactattcacaataatgacttttacactgtcaacaggatccctaaaagccttctctctcccttgagttcctatattttgttcttttgcatttatgtgtttgacacattccactttatatttttattttcactcccacaataggtgaggtctgtgtgtgaataggaattttttcatctgtgtgcctctaagtgcacctagcttgaagctctgaatgttaaacccaaacaygtcttttgatgatagctgagaatgacttaattctcatattattgacaaagtacaattgataatatacatcatatcccataatgacaaatgactaggtattcaaacctacaggctgggaagagttcaaacttttcttctggatcacaggggaaaacctcagggagtttttctacacttggaacatgattctgttaccatataaagtaggaagcaatgaactcaaaagaaagatgtttatcctgatatatctttctcattcctgaaggaatgttttcaccctatggctttgccttggggtgaaacaatgtcaaatgatttaacgcaaatggaaaatgccaaccttttaggttggtttctaaatcctttacatagttacacgtgggagctacatatagatgagctggttggttggcc

SEQ ID NO: 8 - DNA, 648 bp, Homo sapiens
ctgccatttttgtagttggtgagcaaagcaagaaagcaggccacttcctccacatgtttaggaaagctatttcatccatcacttagtaggaaattaaccctggcctgtaccagggagaaagaagagcctgaatgaggtcatgatcactccacatagatcctttcaaccttggctagaaaaatccagtgttgagcttttggtgaagaatcttccaaaatttaggttatacaggttatttatttatacgttyctctatgtctgtgtgtgtctgtgtatgtatttaggttgacagatgggtataacctttttacaaaataggatcatgctatacatgctattatataaccttattttttttggtttacgtttttttcattttgtttcatattctgttgaatgtttattcattcactcaccctatttacaaataaaaaagagaaatattctttccactctcatttatttagtagtagttagagagcaagacaataatcaattaattataccaaatcaatgtgaaggagctctgagggaaagttactctgggagtgcctaacagaggggaccagtttctaaggaaatgacacttcagctagaatctgaagactagcatgtggaggtagaattggggtaggaaaagaggggact

SEQ ID NO: 9 - DNA, 1001 bp, Homo sapiens
cttatttatttatacgtacctatgtttttctttctggaagcttatatgattttctctttattcttgaagttctgaaattttacccaagtgcatttatttatttagagatggagtttccctcttgctgcccaggctggagtgcaatggtgcgatctcggctcaccgcaacttccatctcccgggttcaagtgattctcctgcctcagcctcctgagtagctaggattacaggcatgctccaccacgcctggctaattttgtgtttttagtagagacagggtttctccatgttggtcaggctggtctcgaactcccgacctcaggtgatctgcccacctttgcctcccaaaatgctgggattacaggcgtgagccatcgcacccagccacccaagtgcatttaaatgtgatatttctttactcatccttattgccctcagtgggtcccttaaaacaactcaaacaaaacaaaatacttatttggcttagtaccaaaaaatatytcgaagcaaacaaagatggctcacaataccaccagccagataactccaattaacatttttaaagcataaaataagccaggtgtggtggcccatgcctgcaatcccaccactttggaagtctgaggtggaaggattgcttaagcccaggagtttgagaccagcctgggcaattgaaggatgagaatgggtggagaagaactttattgaacaacagaacagctctcagcagaggggggatgcagggggtagtcccccacccccacagttaggtggtttctctccctgtgtggctgggtccagggcttttcatggactcagaatggggaacgtgtgctgattggtttgtgagtatgcaaaaaaggttaaagcaaggatatcactcaagggtgggcacaacagtgtagaaaaccaattaggaaagggtaggtatatgtcaaataggtgaagggaagggatcaatcaggaaagcatgtcaaacgggatgacaggttctcaatctg

SEQ ID NO: 10 - DNA, 601 bp, Homo sapiens
agctgctgcgaccacatcagagtctcagcgtgtgtttgggggtgggtttgaggctgtctgttgcagagcagtgtagatgaggtagttcatccggagtggcacttgaccctggagttgcataggaatcacctacaaggagctctaaaaatgctgatgcccaggcccctcgccagaccgttccatctatgtctccaacatagtagcgcaggaatgtggacttttaaaactctcccccaagtgatttgaatgtgcagccaaggttgagaatctccttgtgatggaaccacctcactgttagacygaggcctttggaattctgccgggtaacgtttcctaatgaaccaagcatttgctgcagctgttagtggccgaggtgctatgtcatggtggaggtgtcgccacaccccgtcttgttctcagttttttctcctacagtcaagttagggtggggagtgttgtgcactgaggaaagtttgagtcaaatagtctttaaagtctttctcacagctctcaaagcctttaattgttttttgttcatcacagaggagaaaatctaattaataataacagatttaatcaactccaagtattgcttttaaaa

SEQ ID NO: 11 - DNA, 1001 bp, Homo sapiens
ttgagcccaggagttcgagaccagcctggacaataaaggaaacccctgtctctacaaaacaaacaaacaaaaacagtaagacaagactcctcctatctccccaaaaacagaaaattatttaactttgttaagaattaagaacttctatatggtgaaaagaaagtttttaaaaagctacaacccaagagattggttgcacataaatcaattagtatccaaaatatataagtaactccagaacatcaataagaaaaatacaaacagatcaacagaaaacaacaaaagacatgatgggcatttcatagaaaacatgaatagcaaaacacacaaatatattccatatttttagggaggaaatataagtttaagccgttatgagataccattttatacaagccaagctgatagaaatttaaagttttgataagaccaagtgttagtgaggatgtggaacaataggaactcatacactactgataagaacgtatattggtaaaacartgagacagtatatcatgccagtcagaatggcgattattaaaaagtcaaaaacaaatgatgccgaggttgcagagaaaaaggaatgttttacactattggtgggagtgtaaactagttcaatcattgtggaagacagtgtggcaattcctcaaagatctagatgcagaaataccatttgacccagcaatcccattactgggtatatacccaaagggatattaattattctgttataaagatacaagcacacatatgttcactgcagcactttttacaatagcaaagacatggaatcaacccaaatgcccatcagtgatagactggataaagaaaatgtggtacatacacaccatggaatactatgcagccataaaaaggaatgagatcatgtcctttgcagggacatggatggagctggaagctgctatcctcagcaaactaacgcaggaacagaaaaccaaacaccgcatgttctcacttataagtggaagctgaacaat

SEQ ID NO: 12 - DNA, 701 bp, Homo sapiens
aagcaattctcctgcctcagccttccgagtagcgggtattacaggcaaccgccaccacgcctggctaattttttgtattttctttttagtagagatggggtttcactgtgttggccaggctggtcttgaactcctgacttcaaggtgatccacctgccttggcctcccaaagtgctgggataacaggtgtgagccactgcacctggcctgaatttctttatgtaattgaacacacaattctacacagttaattctcacacaatttatttgacacataggtctaccatttttttttctattttacagatgaaatgatgttcagagaagtagaagtgacttacctaaagtcagctagtggctgattcagactcagattcagtccctatggctaggctagaatcaccctaactatcctattacctttcatcaatgaccacagaaaccgctattgttttccagaagttagaactgttttcagaggtgcattggacattctaagcmttattctaagcttggaaaacaaagtgctggagatttgttaaattagcactacagagtacagagttttgagcaaagtcgtattaaaaactcaataaacaatattcttgctcaataaaagtgcttgactttttttttttttcaaaagtacatattatggtcattaaatgccattaagagaaactaggcttgttgaaatgtaa

SEQ ID NO: 13 - DNA, 401 bp, Homo sapiens
tgaaaagtcattttactgattagcagctgcagggcacttgggtggtttgaattatgaattatatttgagttttaaacaattttttggttataaaaggttaaattttcctcgcttgtctcagaaaggagactaaaaaattgagtaaaatcaagaaacgtgttagcacacatatatacacgtacacacgggagactttcaaarcttttttcttaaactgatgaattaatgttaactaagccttattaagtccatactttaaatattctaaaagaattttgatttaacattttctactttcataattatagtaaggaaaataaaccgtaaattggaatatatatatttagaatttgtttaattaactaacatgaaatgtgtgccaatattatcagttataaaga

SEQ ID NO: 14 - DNA, 739 bp, Homo sapiens
aagccagcagacaatcagttcaggcaaacagagcgaaaacttcacttttagtgataatcagcacaagttagatgctatcatggaaagaagcagcttctagaattaagctgcgaagtcatcttagaggcagaagccctctatttttaaaacgaaagcctcgttttagttctagcgaaggtcgaataggacatgtgtccccttacagatgaacattcaaccctcagtgatgtgaagaacgtaactraagaatataatgtaaaaagaacatttttctaagaggtaaaaagctattatgtttcctgggccagggtctactcagtgaaattcagcctggtgatgagactaaaacgtgtttattatttattccccccacccccatcctcctttctcctcattttagggtcgcaaagatgaatttgtttaaatctacagctctccaagcgcaccgagaacagtgcttggcatacaatagtatccaataaatattgttgaatgaattcaaatttcatctgcagaaaaggtaaccttactgatatttgtcttcaatctccccaactttttaaagatttcaaatcttcagaaaaaaaaggtacaatactaaaacgaataccagtttacttattaacattttgccacattttctctacatgtacatactgtttctgttcaactatttgagaattagttacagaaaccatgacatgtcactcctaagtatttaggtatacagctgagaaga

SEQ ID NO: 15 - DNA, 701 bp, Homo sapiens
ttgttcacaatcaacttgtttctggattaattttgttgtacataaatgatttgatattttatcacaagttagttgatattctatcacactgtcacaaatggcacaacgagctccaaccccatctttgccaggcatgaaaaagaaactaagcacatcacagatgttcaaattgtatggtcttaacttcattttgggtttaggtataagttcattttctgagatcactttttatcaaattactatcattggtggaatccaggtattcttttgccacatcaagatctccagtgtaaaaaatcagcttaggaaaacattactgcataggtattctcttttcagttgcaacagaaaatactttattttcaaaatcttctatttattatttttaagttaacaaaattggtgaaaattttaatacatgtttgttttgttttgttttgtttttttgttgtttttagcagaaagttttcatgctatctctgttgcttttgtggattgacrgaggtggaactttgatagctttatgttgtgttttcatggtcagtcactgaatatatccgtcgtctccaacattgtcgtagagaatatcgctgagtaaatgttagggtactagagtcagcaagtctgtgcttgaatttatagtaatgtttgggtcaatctagggttccacagtggagaatggcacacaattacctctcact

SEQ ID NO: 16 - DNA, 509 bp, Homo sapiens
taacccaagcttaggctcaagagggaatatcagcagagagataggaaagtgaagagtggtaccatgggatggcccaagttcagataccctttgaaggcagggctggagactgtgttcgattgggaatgggagaggccctgtcctcagcctgtggagccactggcccctgtgctttcttgttctaattcacagaaycaggaaagaggagagggtgggatgggaagggctggcacccctgctgcatccacctctagcctttggaaggcgatcaggaagctgtgctgatggtactggacgggcagctgctgactgaggaagcgatggctctgtgcccaaggtgggctttgtgaccaggggaagtcctttccagaggaagcccttgaagccaagtatcctataaggggaggattctgtgtcttctttttcttactttccttttttgagaaagcctctagactcgtaccacccgagtcactgtttatttagcgctcttgcaaggctggctcttg

SEQ ID NO: 17 - DNA, 201 bp, Homo sapiens
tctctgtaacctcagtccctgtgtcttcagctctgagcctccctccttgaatgatcctccaagttcctgtcctgacctcaggaggaaaagggatgaaagayagagaaaaggaaaggaaagatagggaggagagaaggcagacacataagagtaagggcaattgagggcaaggacctgaaggatgaagacaggggaacaaga

SEQ ID NO: 18 - DNA, 782 bp, Homo sapiens
cctctggctctgagttctcttttaggatctattgtctgcatatatgtcattgactcagacataaatctcaaagtggcccatacaatatatttgcataatgaattaaataaaataaattatagtaaaaataggcaggtaggcaggcatggtggctcatgcctgtaatcccagcacttgggaggccaaggcaaggcaggaagatcacttgaggtcaaaaagaccagcctggccaaaatgacgaaatcttgtctctactaaaaatacaaaatgttagctgggcatggtaacatgtgcctgtggtcccagctactcgggaggctgaggcacaagaatcacttgaattcgggaggtggagggtgcagtgagcttgaactcaggaggtggagggtgcagtgagctgaaattgagccactacactccagcctaggtgacagagtgagactgcatctcaaaaaaataaattaaaaaaggaggaaaaatggggtaataaaatagatattcatgtcaaaataatatgatttcaaattaaacactaggtatactaattcttaacatgtctcttggccacacaaccaccacctkttattttgactcatcattttacataaacaccacccacatagagtctctttggtttcactctgtcttttcttgtgggactttgtttaattgttcaacaattaaacaatacctgtttaccatctcagatgtaaaagttaaatcaactgagggggttgtttatgtttctgatgacatttatttttgtgatatctaatagtcta

SEQ ID NO: 19 - DNA, 601 bp, Homo sapiens
ttcttagtcttcagcatcagaattctgatcttacccacctggaataatttctctaaatcatacttttatttttatagtaatttgaattgggttagacaatgctgaatgatagcatcaaaaacagagaacttcaatgatatacaaagaaagttccataagtattcatgagtttgttcagcacgtcaaggcagtgttaatgagattttgctttaatcactcccttcctgttacccctgcagacccccaccaatcctccctcactgtgacctgatcatttattttcagtgacaagcacagctckgcctccggcccagtaaatgctgagaaattgtcagatattgaagttgtcctggcctgccccttcagtgaaccacacattaacattgaagagtctggcagcagctgcatcaccttaaactagggaggctgaatgtggaattgtcctcgtcctgagagaggtgttgtcacctgatgtttccgggttacctggtggttccttggtcaggaacatgaaaggacagcattccctgaacagattctccttgaatttcactgttgttctgtacttcaatattaacttacagctgccttgtggttatat

SEQ ID NO: 20 - DNA, 674 bp, Homo sapiens
tgtttcattttgtttgtatttctgatctttagcttgtctgttgccaaatagctctatggaaagagaacaggggctttgaaggcaaacaggttgaatcctagctctactgataatggctttctgacctctgcaaagtagttagtttctctgcacctccttttcttcatctctaatgtgagactaataggacggttgtgagggtcacattgagtgggctataagccaacttgcgaggtgggcagttagaaatgtgggctcccttccctttcctcttgagaagcccaattctagagagcttttcccaagagggaaagcatcttgatygcagttatttctggctggagtagaggtggcaggttctagctatcataactcaaagaaactttagggattatagctatcatcatagaatattaggctcagaatgggcctgagagatcttttagttgaaaccttctgtattgatgagaaaactttgacccagtgaggtcaaagactctgaatcttagagctataagagacttgagtagtcagtccctctaattcaacttgcctcaacgttcctgtagaacatacagcattataaagttattgtttaaagtatttcatctctctgagaatacttatccctactccatggatatgggaagctgaataaatatgggagtcaagag

SEQ ID NO: 21 - DNA, 601 bp, Homo sapiens
ccaacaacccataaacttggaagaggatccaaaacctcaaataaggtgataataggctgcctggtgagaccttgagccgaagatcctgtcaggtcatacccaattcctgacctatggaaactgtgaggtaataaatggccattgctttaagctgacaagcttgtggtacttggttatacatcatggaaatgcatggtaattcaatccaaggttagatcaccttttgttaacaattagttacaaaaacattttagttcttaagagatttctgaattttgaaattgtgaataaggaattgtakgcctgtgattgtatgacttcataaagtatttaattgctatgagctttagttttctaatctttgaaagagaggcagtaacaacacctacctcacagggtggtggtgagaacttaataaaatagtgtatataagatacttgatataaaacatgaaaccaccatcccactgcaaacgttttattattattattattttctaccctttgctctaaaccagtgatctcaacactatagctacatgttaaaatcaccaggacagctttaagaaatagcagtgcctgtgccccactccatatcaatc

SEQ ID NO: 22 - DNA, 3747 bp, Homo sapiens
agaaagtagcccagtctaataatacagcagttctcaacatattctttctgctctgatatttgactggagacattcacatcgctgatacttctctaggacatcctaagaatgtgtcacagacaagaaagtctgccgtttatgtgtgactcctaccacaccaacttctgtttcccatccaggaaatgtgtaaaaggatattacaggtggtgtttcccttatccaaaatgctggagaccagaaatgttttgaattccagattttttttggggggtggtgggtggcatttttggaatggacaatgagagatcttgaagataggaccagagtctaaaaccaaaattcatttatgtttcatctgcatcttatacacatagcctagaggtaattttatacagtattttaaatagttctgtgcatagactggtgtgattgccaggtgcgacagcatgactgtagtcccagctacaacggaggctgaggtgggaagatcgcttgaggccaggagttagaggctgcaatgagctatgatcgcagcactgtacaccagcctgggcaacagagccaaactctgtttcaaaaaataaacaaacaaattttgtgcatgaaacaaagtctgtgtacatggaaccatcagaaagcagaggtatcactatctcagccctcacccccatgtggacaatcagtggttgtgtggcatcaccctcattcctgactgtgacttttttttttttttttttgagacagtctctctgtcacacaggctggagtgcagtggtgaaatcttggctcactgcaatctctacctcccaggctcaagcgattcccctgcctcagcctcccaagtagctgggactacatgcatgtgccatcacgcccggctaatttttgtaatttttagtagagatggggttttgccatgttggccaggctggtgttgaactcctggcctcaagtgatctgcccgccttggcctcccaaagtgctgggattacaggcgtgagccaccgtgcctgcccgtgactctgaatttatatgctactaccaagcagtcatttccttacacttattcacacataagtacttcacagtaaaaaataagacatgccattaacacagtgaagacttacgtgctcaggggagctaagcagccagtggcatcagcagagaccgcaatcagctgctgaacaacagcagcaacaacaaacaacagcaggcctctgctctcccaaccatgctgtgctttgattcaaagcttacgggacacggcattttacattttaggtgagaaggaacataagaagcagttacaggcccaggaagtgggtcctctggggacgaggaggcattctgctggggggctttttaatggtatctgcaccactaacaatggtctyagaaccgacagctcctttctgttggttcagtgtacacaaagtttgtttcatgtacaaaatatttacttagttttttgggtttttttgagacagggcctcgctgtttcccaggctggtgtgcagtggcatgatcacggctcactgcagcctctaactcctgggctcaggcaagactcttacctcagcctcccacacagctgggactatagtcacgtgtcaccatgcccggctaattttttttctttcttttttgtcttaggattctttctttgattttataagctgacatgattgcttgttctgttgtgaatgcacgctgctctgggcctcagttaacccatcacacctgagctccacgctgtccacgggtgctttttctgcagtgttaacgtcatcttcatggtcggtatcatcacgatcaccttgattcagaaccattctggctatttccccaacagtcaatgaaatgaacaaccggagcctcattatcaatgttaaaaatgacattaatgtgtacttcttcccgctgactgatgaactctgaaggtatattttttggaggtcagacatccttttcttctcacttgacaagctgaattcttctaaaccaccacctggttcatcatcatcactgaacacagtcgtaggccagaggtcacgccaggcacgcacatctgtgtctttagtcactgtgttgcaagcgttggcaacagcatgtatggcatccttcatgctcagctcctgaaaatcttctacacctacacctccgttcactgctgcgagcgtgcaattcaagacagtgtttttatatttacttttcattgatctaaagataccctggttacaggctcaactaatgaagtcacgtttgggggaaagtacacagcatcaatattatctttgatgagaatttcagctggaggatgagcagaacagtagtcaaggcataagaaaatcttgctgtcatcatccggtccaacttttctgcagcgagcacaagaggcctgtacgaagtgtttgtaaaaccgatcagaaaagatgtccctggtgatctatgcctttttgttagcataataatggactggtaagaaatttactctttgaaaacagcacggacaaaagcttttgcccatgagagcaggtttacacttatgcgtgcctgctgcatttgcacagcactgcacagtcattctgtccttggcatccttaattcctgtaggggctgtcccgtcagctgtagtcagcatctttctggggcagtagcacccaaatggtcatgtctcatcagcaatacagacttgttctggcatcagattttcattagcgataatcttggcaaactcgtcaatgagtttcgacgctgccttgtgatctgcagatgctttaaaacttgaattccatgtcatttcttaaatttctgcaaccagcctgttgaatattcacagttcccttcaattttcagttcatcaggatagatcttcgcttgtttcatgatcagcctaccactgagtggcacgggttcagtgtgatgccttcagatctagtttttcaatacactattcacatcttcatttttagctttacgtagagtttttctatttttcattaacttctgctcatcacattcagcatagaacttcaaaagcgtatccttctgtttcttcaggtcatgtatggtggtcatcccaacaccatactcttctgtaagacgcttcacacttatactgctgttcagttcctccaacaagttgactgtttgtgctattgataaacataaattattcctctttttctgatcactgttacacatagggggatctgcaggccttttcgacattttcagtaatatttttacatgacactgcagagaataagcaaaaaaccacagtgagtaatgcatggagttctcggccccacgtgaggtatcgtggggaatctgcgttgggtgcgtccagcctgcacacgtgccattttattacacattgtgggcgtgcttgtgtgggggaatttgggtgtgcatggaaaagttatactatagttgaagggggctgggtgggtcttttttttcccttggggacgctaaatcaactgtgtcgtgcacctgtgctttgactatgccctgtcacatgaggtcaggtgtggaattttccacttgtgttgacagattggcactcaaaaagtttaagatatcggagcatttcagattaaaattaggaatgttcaacctgtattacatggctagccttgaatccactattatttactatgttttctgttacattttataattgtttatcattgggtatacaaatgctaaagatttcagtgagtggatcttacaaagaggaatttt

SEQ ID NO: 23 - DNA, 501 bp, Homo sapiens
tctccagaggatatttttcattcaaattaagaaaaatctgacttagattatatttctttaaataaaacaaaagattttaaaacattttcacaaactaaatcaaatatgttaaatattatatattgataggatattttagcaacaggtcaacttctacatgttactttgaacaaatgtctttatttcccaatatgatattagattaactaaaataggaatgtttttgttaaatattattgataacccccacrccttccacatttcaaaaactgtgaaatctatgtagttccaccaaagcctaggttttttgtttgctattagatgttcttaagagatttttgcactatttcctgcattattacccttctcttgatcatactattttttacagttgaaatgctaacacagtgaagttttcttaataattggaagctgaattatttgtaaaattcatgacctcactgctatcaatatttaataaattatactatgcagtatcat

SEQ ID NO: 24 - DNA, 701 bp, Homo sapiens
agctgtgtccactctggcttgtcatttgataacgctactaacaggagcataataacatcaactcttgactgttttataaagtgtaatttttttatttcgatttttaaattttaaaacatataaagattatacttccaggttaaacctttgtctgactattccttgtacccccagaggcaaccactattgtgtttttacatktattgcttctatgtatgagtatacttttcctattcatgaatttatccttaaacaggaaaatggataagtaactgggatacttaagtcttgtattagagagatgctttggagacagaagttatattagaaaaggaggctgggcgcagcggctcgtgcctgtaatcccagcactgtgggaggctgaggcgggtagatcacctgaggtcaggagttcgagaccagcctggccaacatggtgaaatcctgtctctaccaaaaatacaaaaaaaaagaaaaaaaaaattagctggacatggtggcgggtgcctgtaatcccaccttttgggaggctgaggcaggagaatcacttgaacccaggaggcggaggttgcagtgagcagagagctccccattgcactccagcctggacaacaagagcgtaactccgtctcaaaaaaaaaaaaagaaaaagaaaaagaaaaagagattctagagacaggaagattaattagaagattgtt

SEQ ID NO: 25 - DNA, 601 bp, Homo sapiens
ggtcagttatatttgggatctgggtccgggatacaaaacttggagtcagttcattgcacctatttgctgtttgaccttagaaaatttatttccatttctaagtcttggtttcatcatttctcaaatgggaataatcacagcctgctaactaacatcacagagaacttttaagaatgaaaatgaagtaacatatgttaaagcacagtaaagaaccaagcaaatgcaagacttttttttataagaaggttctttttaataagacgtcagtactcaaagagttgtcaatgaggatagatctcartatcatgctactgaaggagtttgtgtagatggacaatgcctcctgctccaagagcaagcacatggagaaagggtcaatggacacatttgttttgctgtttaacaacaaagaaaaattatgaattagacaaaatgtagtctagatagaatttatttttgtgaacattaatgagggtttggtcatgtatttattaaaaccatgtgttacgccatgcccggtggctcatgcctataatcacagcactttgggaggttgaggcgggtggatcacctgaggtcaggagtttgagaccagcctggc

SEQ ID NO: 26 - DNA, 601 bp, Homo sapiens
ttaaactgtggctttctccttttgtacctatacttaacactccttaagttgattctcttagtgtcaaacttgttgaaatttctcattggttatctctctgtatttgacattgttctaacagtcgtataatctgttgctgggtgtttatatgtagtattttcattacaatatcatacttttgaagagcaaatcaatggcctgggaaaaaaatcagagggcatagtgcctcattacctctttgttaatgtttttctacctcaatccttacaatttttatcccgtgtatggacttaaaatagtrtgtgttgttggttgggggaagatcataagtgaagacaagagagatgaagtaaatagatagagtttgacaatggggcattctgggggaagttaacaccatggagtcgaaaactttgttagatacttagatatctggaaactttaggcattaatgtgtgtgaatatttctatatgagttcataataacgtacaaaaagaacccgcaaagcaaacagacttgatgctgaacttgtttataccttccaaagagccaaaaattgagaaggcactcataaaaattaatatccaacctccactttca

SEQ ID NO: 27 - DNA, 2009 bp, Homo sapiens
gtgacattcctcattgaatgtcttttatactgtttgtgtttttaaactttgtaactttaatgcctgtaaagaaatcagtttaaaaatatgctgatatggtttggctgtgtccccactgaaatctcatcttgaattgtggttcccattatctccatgggtcctgggagagaccaggtgaagataattgagtcatgggtcctgggagagaccaggtgaagataattgagtcatgggggccatttcccccatcctgttctcataatagtcagtcagttctaaagagagctggtggttttaaaaggggcttcccctttcactggacactcattctctttcctgctgccctgtgaagacgtagcttccgccatgactgtaagtttcttaaggcctccccagccatggggaactgtgagtccattaaacctctttcctttacaaaatacccagtcttgggtatgtccttacagtagcatgagaatgaactaatacatatgcctaccaaactgtcgcagccaagcggagctaaggagatggacaactcagtgtgagatggtgaccaagatgctcttaagcttcccatcagcttgactaaacaccaggcaggcttctccagtctctagatccctgacctgccttttcttaaagcatttactttagaaacttgcaattgtaaattctttctctgcccctttaagacataaatcttttataaagtttcttgccagttttacaatctaggactgtctttctcaaggacgtgggagctatttctttgaaatgtaatcatcaaggaagacagtacccctatctcttagtctttgtggaagggtggaagcccaacttccatggacaccaattagcaaacacagatggcctaatcacagagaaatacatttgcaaagtcaagaataactcaatgtgctggacatatcctattgttcaatctcctaatgtcctccagtacttttccacttactccagcaattaaaaaccctcctgtccttttcagtttcagtgaaattgagttcagacctctctccttcctctattgcaatagccttgaataaagtctttcttgcctgtttaacatcatccagtgtcattttgctttgatgtctgtatgytgaatgggctcctgtatcagaaaaaggacatcagataaaaactaaggaaatctgaataaagtatgaactttaggtaataataatgtatcaatattgatttattaattataacaaatgtaccataaaaatataagaggatgctatgggaaactgattgcagggtatatggaaactctctgtactatctttgtggtttatctataaatctaaaactgtttaaaataaaaaaatatatttttaaaatcctatctgaacctggaggacatcatgctaagtgaaataagccaaacacagaaagacaaatactgcatgttctcacttgtatgtggaatctaaaaagtcaaactcgtattagagagtagaatggtggttatcagaggcagggggtgggaagaagatgaggaattgcggggagatgaagaatagggagagattggtcaaagggtacaatgtttcgattagacaggaggagcaagttttagtccattgctcagtgtggtgaccatcgttaataatggattatatatttcaaaaatttgctttgaaaagttttttaatattctcatcacaaaaagtatgtgagatgatgaacatgtttattagcttgatttaatcattccacagtgagcacatatgtcaaaacatcacattctatctcataaatatatagaagtagtatttgtacattaaaatatatattaaaaataaccctatctgagtggaatacggaaaagaacatccaagtgtcagaaagatccagtaagtaaggctctgacaccagctatgtgatgacagtcaagtcatcagatctctttgagacccagttacctcatttataaaatgacacttgcaaatccactaacatcatgcattctcagtagtcgc

SEQ ID NO: 28 - DNA, 1000 bp, Homo sapiens
gagccgtggacccagcccaggggagggggttagggaatgtggtgcacacgaggcctggctaagctagggctcaggacaggacttgtggcaggaaggatcccacgttgttaacccaagacagcagataggagttgttgcaggtccctcttccggaagcacctctgaaacggaaacggtgtgtgcggtgtttatcaggaagtgctctcagactccacgcccgtgggagtagtgagaggcataggatgggacagaaggagatgctaaacagtgatgcagaagcagcgaagacctccgcaatcctgcagggaagcttgggagctggtacggccctgcagagatgcactgaggtagggcgaggaccccggccctctaatcccccgcactgactcgacattggatgcacagcccctgggaaggggatgtgacgggcgaggtggctctcggcatctgagagcaatccctggcgacagactcagctgagagctcatctgagggcaatcyctggagacagactcagctcagagctgtcagcagcctgaggactgtgtttcttcttggacgggtagggaccccaggggaacccgggcagcacacctcacgccccctgcaggcacagtgctgcaactacagccaaccatccggaggggagaattcaggtgcgagaggcccagggaggaagccgagggagactccaggccctagtgagaaggcagcgcacagccagcaaggcggacccacctagctgggtcctgcctgcagacatcgtagtcagcatctgcggccgtggcgtcaaccagtaggaagcggattaggacccaaccaggctcagccaggctccaccacgctaaggttctgggctctcccagcatctctgaggactgagggaggccccagcctgggcagaggacacctctgaggtcgtgcaaatagttttctatgaagctcgtcttctgctgctcttccacccattgtccatcagtccttgccacttccttgtcct

SEQ ID NO: 29 - DNA, 2937 bp, Homo sapiens
ccagttagatctaacagatatctacagacaacttcaaccagttatagtagaaaaagtattcttcttaggtacacatctaacagcttccaggatacatcatacatcatgctgtgaaaaaaagcctcattaaattcaaaaggattgaatttatgcaaactgttttctaaccacagtacaatacttgacaaaaacatcacaagaaaattacaaatcaatatcacttagaaatatagatcttagagtcctaaacagaatacaactgatacgtacatgcaataaataaattatacccctaccttacaccatatccaaaatttaactcaaaatgaattatggacttgagtaaaagagttaaatctataaaactcttagaggaatacagaattttaaatctttatgaccttggattaggcaaaggtttcttgtgtatgacaccaaaaacacaggtaactaaagaagaaatacgtaagttaggcatcaccaaattaaaaacttttgtgctttaaagaacaccgtcaaggaagtaaactgacaacttacatacagaaggagagaaaatatttgcaaattacatatctagtaaaagtgtagtacccaaattatgtttaaaaattatacctcagaaataaggtaaattatatttcaaaatagacaatgaatttgactaaacgtgcttccaaagaagatataaaaatggccaataagcacacaaaaagatgtttcatatcattagtaattacggaaatgcaaacccaaaccacagtgagctactactttgaacatgctagaatggctaacatgaaaaaggcagataatatcaggtcttggtgagaatgttgagaaatgaaagcactcataacttttgtgtctgtttgtgtagccatttataaacaaaaagtggtatatccatagataaaattgtatctggtaataaaatatgtgcagtactgatacttgctaaaattggttaatccttgaaaacattataagtaaaagaagctggctaaaaaggccacactgaatactggcatttatctaaaacgtccagaataggcagatccatcaagagaaagtacactgatagttgcaaggatctggccaagtgtaggaaagagaatgactgtttcttcacagtgagttttttggggtagcgatgaaaatgttctttagtgatctagtggtgccgattggacaactctgtgtatatattaaaatacactgaattgtatactttcaaaggctgaattttatgatatatgaattatatcttagtaaagctgcttttccaaaaattctaaagttacaggcccatgtaaggcatagtaattcatgaaaatttagaattgtttaaaaagttttatttatatatttatgcattgctcaatcagcatgcataaactttttatagtgttcaggttatgtctttccctctttgggtccaggatctatttctttaggttctacattgccaaagtggtattctttgatcattttgcatgtctcagcatgtcataaaaaggtagtgatgctgttatttatcacgagccatgtctcttaggaacttttttctattgcaatagtagtgatattcttgcctttgctgttaatgaacgccatttatatactgctggttgtatcattgctcatgatgctgtatcacctcttttgcctcccatgaatattgtcttccaatgtttctatcatatgttatttctttattcttttagtgatgaccgaaagtacaagtcttactcagagtatgcaggaaaatgtgagcaatacttgttttctggtcgtgttaaatttagcaattggaatttggtaacataaatataaaacaatccatgttctttccatatctcaaaaaatacatacattctaggatgtggacctttccctaaattaaccatatctccttcttccaaacactacaatcaggaagaaagaccagtgaaaaactgcacgtagactgagaacagcagtttcatttatacaaactrtatccctccctcatctggcttggcctcagattgacaaagcagaccatgatgtcccagttggaagtaagagaggacatcacttggctccctccaaaatcctcagaaaagattaacatagctgccactagaggactgcctgacatatgtcctgatctttaaagaacatgatgactgtcttctatgcagtgtcaaagggcacagagagtaaaggcctcctaccaccatttctgccatccttgagggctgtcgttcaaaggcagtactgcagtgacaacactgacaagtgagagttaaccaaccagcaagaactgatgcatgagtctacgtttatagctcaaacccaagacatagtggatgattgctagccaagaaccatgcattcatctcttgtccgacatggaagagtcacctaaaatcagcatgactgtaccattctgcctgtccaatttcatgcagtttcacaaaggaaatataatgtaagtcagagaaaatgattcacttgcgagtccgccttcttaaaactagagaatggccaggagatttaaagacaacctcccaggtgcaagtcctggagcttgttagagcatctagtcaagtgcacatgtgagactggggagttagaagggttggagagagtcttcaaggagtgaaatggtgactgaggctcacatagtttgaagtgggattggccactcatcttaaatggcaggccagccctatctcatctcaggcactgaaatgggatttattaatagaaacttttgagaaaggcattccaaagaacagaggtgaagtgccaaggacaggattcttgtttgggtctaaaggcaatattgtttgaagtataagaattttagatgaagttctaatgttgctattttaatcctttacttctccttagaatagaggaacaattatttcaagcactggacaagag

SEQ ID NO: 30 - DNA, 851 bp, Homo sapiens
gtttgcaaggacatctccctggttattacctttacttatattgggattccactttaacaaatgctgtggtattcacctatatttgttttcagagtgtgctatatttccattatgatttcctaaaaatgcctttctgcctcctctatatgttctgcttcctgaatcatttaacaggtattatactttgtgcacagacacacctattttgtttattgatcagtctgtttgctcctgttctcatacctcattgtttttattcactcgttttgtatatgcttaaaatctggcagggaaagtcatttagctttgttcttcttttcaaaattagaatagttaccagcaaatctacaytagtctatataaatttttaaccttgtatgagtattgaaatttacatatagaagtgtacctaaaacacgtgtgtgtgtgtgtatatatatatatatattttttgagacagagttttgctcttgtcgcccaggttggagtgcaatggtgtgatcttggctcaccgcaacctccgcctcctgggttcaagcgactctcctgcctcagcctcccgagtagctgggattacaggcatgcaccaccacgcctggctaattttgtatttttagtagagatggggtttctccatgttggtcaggctggtcttgaactcctgacctcaggtgatccacccgccttggcttcccaaagtgctgggattacaggcgtgagccaccgcacccagccacgtgtgtatatttaaaagaattattataaacagaacatgtatttaaccaccattcaaatgacaaaatctaacttaagcgtaattaagaaaacacctgtgtgcaacttcagaataa

SEQ ID NO: 31 - DNA, 601 bp, Homo sapiens
tgtgccttgggaagagctctgggcctctgggggccgtgtgccctcccgggacatggctgtccctcttcaaaatgtcactctcttcgcccaccctgaattccagtagctgcctcttgtgcacaagcatagccgacaggctcaaactcggcttagctagatgtggacaggtcgtggctgcctgccccacgcaacacagagtctgctaaatgcaggcacctgctgtgtgtggtgcacgttgttcccagtgggtcaggctgcctgtacagggaggtcctgtgttccctgggcacgtctgtgcatktgtggcaacatgattatgcaggccctgtcacctgtttgtgtgcccatccctgcacatgcagctggctggctcctatgaaaggaggagggtctggaggttttggggtgtatagctttcctaggcgatatgtgcagtgttaacgtgtgcattggctggaaagtgtgaaagggaggcggctcaattgggttcggccggacagcatcttaacataggtctcagctgctgtgtgtacaggaatagtgtcccaggggtctgcagtgtgtggcaagtattctgaggtggggttgtgagaagaggcag

SEQ ID NO: 32 - DNA, 757 bp, Homo sapiens
gctgttgtttcctaatgtcacctccctcagtaaaaaggaatatacatgatcaataaatgcgatatctttaggaacacaaagaaaagggataaacgaggaatatataagaaataaagggagggatagcattaggagatatacctaatgttaaatgacgagttaatgggtgcagcacaccaacatggaacatgtatacatatgtaacaaacctgcacgttgtgcacatgtaccctaaaacttaaagtataataaaaataaatcaataaataaaaattaaaaaataaataaataaaaatcaatattcatcagaaaaaaagaaataaaaagcaatctacaaaatgtttaaaataaaaaccctaatctcaaaacaatgcgagaayaatgtcaggcacaagaagcaagaagcataagaaaaactagaaacaaaaaaaaaaaaggtgttgcccaatttgaacaggaaaacaatagataactcaaaaaagctgattcttttaaatcaaaaattagccttaagagattttttagtgttatttaccgagcactattctaagtgctttacacttagatccattacacttcatcttcacaaaaccctcgaaggatggtactattagcgccgctcccatttcctagtgctgaggcgcagggaggttaagttaattagggcagtgctagatctgggtcttgaccccatctgaagccagcacctgcaagcatcactactgctaatacttgccatccattctttaaaagccat

SEQ ID NO: 33 - DNA, 401 bp, Homo sapiens
cccctatagaagtaaatcttcacttcaccacttatccttgagcagaagaggactgtagttgatgattcacatgagaggatgacaattcaatctcaattagcgtagattcttgaaacatttccaggttgaagtagctattctcagccctccataggctggactgtcctccccagaggacttcttagaattcaattctggccyatgagaccaacaccagatctaagcctagctgggcgtacctgaagcctcagcatgccccaatgtaacatgcaggcgtttggccatatggtgtcccctgagcttgccgttctctgatttatgccagaatgggcatatgctgggatatttcaggaccctgaggggagagtcaaaatgtcacacagccagtatgaggttaagct

SEQ ID NO: 34 - DNA, 5361 bp, Homo sapiens
atatatcttctgtaataagatatagatatcatctgtaataagatatatatgtcttctgtaataagatatatatatcttttgtaataagatatagatatcttctataataagatatagatatcgtatatgtatatgatatatatataatatatatcttattacagaactccgaagtacatgaagcaaaaactgacagacataatggagaaacagaaaaatggacaatggtgattttaacacatctcttccatcatacatatatatatatatataaaaccttgaataatttatatgttatgtatgtatagacacacacatatgtatgtattatatataatttatataattatataaatatataattatatattatataaatatataatttatattatatataacttatatattatataatatataatttatattatatataacttatatattatataatatataatttatattatatataacttatatattatataatatataatttatattatatataacttatatattatattaaatatataatttatattatatataaattatatattatattaaatatataatatataatttataatttatatattatagataaatatataatttatatatacataatgtacattaaatatacattatatgtatatattatattatataaatagatataaaatttatatataaaatttacatattatatatgtatacacatatatgtgtgtattatatatatatgtataatcttgaagaatatatataatcttccagatttttctaggtagacaatcttgttttttacgataaggagcaattatatttctttctagtctgtgtctcttatttatttatttttggttttgtttcattgcactgggtaagactactgataagattttgtatagagtggtgaaagcagaaatccctgtcttttacacaatcacagaggagagaaactttaagtctttttttcactaagcataattttagcagtagttttttgtttgttttttgtttttgtagatgccctttatcaacttgacaatgtttccttctattactagtttgctgagagtattatcatgaagggctgttgaaattgccaaacttcccatgtgccaaaagataataataaagctyttctttttgcttgatatgatgaattgcagtgaactctcattatacatagatctgtatttgtgaatatatctacttgctaaaatttatttgtaagcctcaagtcaaaacttgtggtgcttttgctgcatttgcagacatgcacagaggagtgaaaaatttgagtaacccaatgtgcacatttccagctgaggtcgacaaggctatattctgccttgttttagctgtcatactatgaccaagcatcctttttatggtgtattgattggcacattttttacatgtttgtgcattttgttaattattttgctgtttaaaggggcccccaagcataccaatgaagtactatctagcgttccttacagagagaatacgtgtgttcaagctttcttcaggtatgagttaaggtgctcttggatatgagttcaatgttaattaattaacaatatatattaaataaagtgttttgaaacagaaacacacatccaacagggttatatttgatcagttgttgaaaatgttgtgaccagaaactcagaggaacctaatcttgcatttcttctaggagcaatgattcagtattcgttacttcaatgttgatgtacactttatagaacataactactatgaatgagcatcacttgtatactaactgaagtttgaatgttgaaacattcttacattcttgaaatgaatcccatttggtgattttgtgttactctttttacttcttgttgtatttcatgtgttggtactttatttaaagtttttgtgtgcatgttcccgagggatattattctttagttttcttctttgtaagtttttctgttgtttggaattagggtaacattggcctcataaaatgagcgaggaggcatttccttctcctctatactctggatacatttttggaaaattgtcattatctcttccttaaatattagacaaagtccatcagtaaaaccatataagcctccagtttactttgttggagagtttttaagtatcaatttgattatttagtagatttagaaatattcagtttatctatttctttttcattgatttttgagagttgggagtctttcgaagaattttattatttcatctaagtttttgaaattatggatgtagagttactcatacatagtattattaaccatagtgatagcccctctttgatttccggtattcgtaatttctgtattttctcttttttcttgattggcctcagttttattgatttgttattaaaggaatgagattttagttttatttcttttcttttttttgttctcattgacttctgcttttattttttatttattattttcatttttttccttctacttgctttggatttaattttctctgttatttctatgtttcttaaaatggaatcagaatattgatttgaggtattttctctcacgtaatgtaaacatttaacatatgcattttatatatttaatatataaattctcataaagtgctgctttagtggcatcatccaaattccaccatgctatgtttttactttcattcaattcaaaatattgttaacttccctgttatgttgtctttgacctccatggcctattagaactgtattgtttaatttccaaatatgcagagggattttctaggtatcttctggctactgatttctaattttaatctattattgttacaaaacatgtttcctagggaattgttagagaattgtttgtggatatcaatcatttaattatgttgaggtttggtttttggcctaaaatatggtctttcttgaagaatgtcccatttgcacttgaaaagactatatattcagctattatttagtggcatattctataaatagcaaagaaatgaagctgatttataatgtttttcaggtttactaagtacttactgaatttttgtctatgctttaattttctgttgaaagctcacatcttgtgtaggacaatagaggctgaggtaaatcgactttatgcttggaaattggtaaatgggcatatattttcttttgctagtcctctgttgtggaaggctgagtcaaccagttaggaatgaaatggtggttgggctcttttgttgttatggtaaacccctgaccaccacagacttagaattcctctagcattaccttgtgtttaaggtggggttggtttactacaaggattctctcaatatttgctttatcctctgctctaggtctttcttttgaacttgtgcctcagccggggtctttcttcacccttctttgtccctttccaagcagaagccgtctgtcacttggtgctgctatagtggtggtataggctggaagagaaagacaccctctatggttctggtcaagcctccatcttaagtagacactgtccctggattctggtgggtgggctgtctcgggatttctgctctcctccccctgtaggtttcagcgtggaatttatttttctcctttctcaggggtaaaaggcattttctgttttcttccttcagcagttccagtgccttgcggggaatgtcttcaccagtgctctaaaaggcaacaggattttctgccctgtatccagcagcttaaggcttttgtttcaaaagggaataagagagaaaaatctctcctatcatgcttttcttgcggtactgttgcctgtttttaactttttgtataaatggaatcattcagtatgtacattttgtatctgttttctttcactctacagtatgtttgaaatgtttttatgttgctttgtatatagttttcttcagatttctgaaagtatgaccgacaaataaaaattctatatatttagggcataccatgtgatgtatatatttacatatatatggaggcataggggaatgattaccacaatcaagctaataaacatatccaacacctcccaacatctctcgtagtcactttttatttttttatttttatttttttggtgaaaacacttgtgatctagtctcttaaaatattccaccagttgtggtggctcctccctataatcccagagacccagtaggccaaggcagaagaattgcttgaggccaagaggtcaagaccagcctctgccacacagtgagaattcatctctaaatttgttttaacaaattaactgggtgtggaatcacacacctgtagtcccagctacttgagagactgaggcagaaagattgcttgagcctagggggttgaggctacagcgagctatgatcatgctattgcactccagcctgggtgacagagtgagacaatatctcagtgaaatcaactatacaacccattattattaactatagtcaccatgctgtacatcatatctccagtgcttattcatcttatatccaaaggtttgtatcctttgacaaatatctctctttcttttccttatctccagaacctggccaccactattccacctattctgttaccaggaattcaatgtttcttttagattccacatatgagtgagatcatatagtatttgtctttgtgtgtcttgtttatttcacttagtataatgtccacattgttgcaaatgataggattcttttcttttcaaagactgaaaaaaaaatttacttttattgcttctgtaccatggctattgtgaatagtgctgcaataaacatggagtgcaggtatctttttgatataatgattttatttcatatggattttattttctttatacccagaagtggtattgctgcattgtatcttagttctatttgtaatgttttgtggaacttctaaactctttttcataatggctctaccaacttacatccctacctagtaaggaggtcgaattcccgatagctttgactgacacagctattccccctgccacttgcagttctcccaataaccgcagaatgtaccaaaaaatatgacatcttcagataaggataacaaccttatccttatccgtgcctctgttgcttagagaacaggatgttctccagtgcttacactcagtgagcccagatgagcttcatctgccatgagctgcttttctgagtcttgggggactggcttgccatggatcctaggcttctgtttattcttgctgcctgtctgtaaataatacatctgcattcactgacttgtgtgagtgtcctgtttcactggactcatgcaggtggtagagctactggggctctttccctcctttcagctgtcct

SEQ ID NO: 35 - DNA, 871 bp, Homo sapiens
tagagtaggaacataaatatcaataccaacacaaggatctgagactaggcttgcacccatggtaggggtgtaaaacccattcattttaagaaactttcaccaaaccaaactaggaataggaggttactctcttaatctgatagagtatctactaaaaaagctggagcaaacaccatcctatcagtaaaacatgaaaagcattccctttaaccctatttagtttcaaatggcagttatctaataaagttagcacataaggaagagagaaacaatattgtaaaaggaaaaatccagatgaatctacaaataaatgtttcataggacaaaattcagtaaggtggtcagatacaaaatcaaatacatttttatayggcagcaataaacagactatatacactatatacccatttatttaataatcacaacaaaaatacaaagaacctcggagtaaattttttaaaaagacttataagacctttatttagaaaacaatttaaactttgtaaaatacctcaataatagtaaagaggtatcatgttcagaggtattataaaactatgaattatcttcaaatttatccacagattcaatgcaatctcaaataaaatttatcttatttatttatttattttttgagatggagtctcactctgttacccaggctggagtgcagtggcgtgatcttgactcactatgacctctgtctcctcgtttcagactattctcctgcctccacctcctgagtagctgggattacagccatgtgccaccactatgcccagctaatttttttttttttttttttttttttttttagtacagacggggtctcaccatgttggccaggctggtcttgaacttctgacctcaa

SEQ ID NO: 36 - DNA, 701 bp, Homo sapiens
acttgaagtttgagaattgaaaagcagaattaaaaattcagaagaacccattggaaatctttacaatcactaagagagaccagagatctctctcacaaaatacggcataaaagaataggtaaaaatgtgtctatgatgttccaagagaaaatgaagagaaggttgagaagatgtatccaacaatattaaagaatacaactkattgaattgatcaaagatcaaagatatgaatcatcttacagggtctatccagtgtcgagaataataaaacacaaacacatttacacatatatatacttattaaactcattgtaataaaacctaaaactaccaaaagagatacagattactaaaaatttccagagacagaggggcagaactcctttttaatcatacaaatgaatgggagttagtttgctattagtcttctgttacggactaaatgcgctcctaagattcctatgttgaagtctaaagctgacactcccaatctgatggaatttggaagtagggttttttggagataattaagtttagataaagtcatgagattcaggcgggcccccatgatgggatgagattagtgtccttataagaagagagggccgggcgcggtggctcatgcctgtaatcccagcactttgggaggccgaggcagacagatcacgaggtcaagggatcaagaccatcctggccaagat

SEQ ID NO: 37 - DNA, 1001 bp, Homo sapiens
ggcacagtggctcatgcctataatcccagcactttggggaggctgaggcaggcggatcgcctgaggtcaggagtttgagaccagcctggacaacatggtgaaacccccatttctactaaaaatacaaaaattagccagatgtgatgacatgcatctgtgatcccagctacttgggaggctgaggcatgagaatcacctgaacctgggaggtgcaggttgcagtgagccgagatcgtgccactgcactccagcctgggcaacacagcaagactctgtctcaaacaaaaaaagaaaaagaaaaattaaaccccacctccccagacccatccttcctgggttaatctaatatgcttctctacatgttcatgcaacatacatgtacacatgttcaacacatatggtgtttttgcatttctttctttttctttttcaaaaacaaaaatgaaatcactctcaaacactacttggtgacttgccttttttttttgcttaaactttgcrtatgttggccattaccagactgatccatatatgtctactcactctgtttaatagctatataatgtctgagaatatggatatacaattatttgttcaatcagcatcctcttttgacggaagtaatttccaggtttttcatcttgacacacaattccacaatgaaaattttgagacagatgtcatttagtaccactggggcttttcttgtaaagcgtagattacaagcattgccatttctgggccaaaggatatgtgtgttttaagatgtaatatatccaggccagacatgaaaggacaaatactgtatgattccacttatatgaggcagctagaatagcccaatttatagagacagaaagtagaaaagtgtttgccaggggctgggagtggacgggaatggggagttggtgtttagtggggacagaacttctatctgggaagatgaaaacgttgtggaggcagatggtgatgatggttgcacaacatggtgaatgcactta

SEQ ID NO: 38 - DNA, 550 bp, Homo sapiens
atatttaaatggcattgtcttaaactacattctacagagaattaatgtcycataaaatggtagtaaattttcattaaaaatgaaaaaaaatcgatactaatgatatgatttactaaaatatttaaatatatatgcatttaaatatatatgcaagaaaaatattaggcatgtataaatagtagttattatttgagatttactttgaagatataatctgctatacttgagaatgccaggaagtaacatgagtgcataccaaatgcttgtacgtttagtgttgccactgaccattcactgagcatcacctgtgaatgatctactcaatggtcacatgaagggcttgtatcttataatgtccacatacatttgaaatatatttaacttttacatataaataaatcataaatcaaattttaagtttttcatcaactaatttgcctttgagactatctattcttatctacttatttttgtcatttgcgaaaatatactcagctgatctcacttattatatttacagatcaactttaggttaagattgttgagatcc

SEQ ID NO: 39 - DNA, 1932 bp, Homo sapiens
ccccttggaaaaaaaaaccacgcacacaaacaccatcctgcctttttttttttcagggctcatctctggacccacaccccataaccaacatggaagcctggatttggccctggatcaggctggctccccagggaagggtccagtctctccggggagacatcccgacttcgggtcctgctttctctactgtaattcgaggcyggccatgcccctcagtgggcctcagtttcctcacctgtacagtggggaaatccataggacctacctcctgggagggcactgaggaccgagggaatgaatgcctgtccagtgctcatgatcgtgacaagcactgcgcagaataaatccggaataaacgctcaccgggtgagggagggcagggaagatttctaaaatccccctgatttgagatgagatctgattcacccacttaaaaaaccacattgggaaataatttgaaatgtacaggaaagttgtaagaatcgcacaaagaatactcccaaacgttttgcccatactcacctgttttctgtggcaactttgagacagaattcacacgccatacagttgacccatctcaactgtacaattcaatggcctttcatagtcagagttgtgcaactgtcaccagggtcaattttagaacgttttcatcactctcctctcacaaaaaacccctcacgtcccttggctgtcactctgcacctattaatatttttttgtgttaagactttttctttgagatggagtctcagtctgttgcccaggctggagtgcagtggcgtacatctcggctcacagtaacctccacctcctgggttcaagcgattctcctgcctcagcctcctgagtagctgggattacgggtgcccgcctccacgcccggctagtttttgtatttttagtagagacagggtttcaccatgttggtcaggctggtctcgaactcctgacctcgtgatccacctgccttggccccacaaagtgctaggattacaggtgtgaaacactatgcccggtaaatgttaagacttttatgcatcttcttgacctgtggtcttcagcatttgagtcttaggggacctttaaaatattaagataggccaggcacagtggctcatgcctgaaatcccagaactttaggaggctgtggtgagacgattgcttgaagccaggagtttgggacgagcctgggcatcatagtgagacccccatcttggcaaaaaagtaaacataaaaaatattagctgggcatagtggcgagcatctgtagttctagctaattgggaggctgaggtggcaggatcccttgagcccaagagttcaagagtatagtgagctatgattgtgccactgcactccagcctgggcaatagagtgagaccctgtttctaaaaaacaaaagccatactaaaatactctgcaaagacctcatattcatttattcactcaacaaatgtttgttgagcacctgttttatgccaagtccaatactaggagctctggttgcaacagagagcaaaacagacacggccctgccagtcacctggggcagggaatccccctgccccactccctgcaataatcgattgtagagaagaattagcaaatcacagctgagaagggtctgctggaaaacacagcagaggccttgactttgcctgatgaatggaggtgggcttcctggcagaggggcgtctgtgggtgagggaggaagtaggactggctggtcagaggggacggcacacaggaagctgctgagcaggcaggagtgtgctccgtggaggagctaggaggaggccagttcttagctggagggtggagaaggagggctaaagccaagcatgcagaggccgagggatgtgcagcgccggtcagcccacgtgagatctttcctccctctccgtggactgggaacctccatagttacca

SEQ ID NO: 40 - DNA, 874 bp, Homo sapiens
ttccctcccccagaagtagccactatcctaacttgtggaaaccgttttctttctttcctctttagtgttacaactaaatgtgccccataaacaagcatcagtttgtgaatatcatataaatggaattagaccgggtgtggaggttcatgtctgtaatagcactttgggaggccaaggcaggtggatcacctgaggtcaggagttcgagaccagcctggccaatgtggtaaaaccctgccgctactgaaaatacaaaaaaattagctgggcatggtggcaggcacctgtaatctcagccactcgggaggttgaggcaggagaattgcttgaacactgtggaggttgcagtgagccgaaatcacgccactgcatgccagcctgggcaacagaggaagaccctgtctcaaaaaaaaaaaaaaaaaaaaaaagaaaaagaaaaaggaatgatattgtatatattctttgtgacttgtttcttcctcacaattgttcttagacaakgtgtgaagaggcctttttggtgcctgctgatgttttccgggatggtggattcaggagtatgctgtcttcacaaacatttctggcctcaaagtcacattgagcagcttgggcctcctagctcagccacttagtgtgtgacctttcttgtcctcctttgtaaaatcagaacaaattattcctgtagacatgagcacctcaagagcaggagtgttcctcatttgccattctgttctctagaacttgcagtgcctggtgcctaatgaatcctgagtgaacacaggagacatggtgagatcatgtatgtaagggcctaataccacccctgatttatagcaactgctcaatacatgggagccaatcttattattgccac

SEQ ID NO: 41 - DNA, 701 bp, Homo sapiens
cactacagcaagtggcaacaggtggggaggtggtccagatgtctttcggcttcagagttggtcctggacttgggttctggagtctccccagtgaccaggatttctcagagggggtcccaggtatggacactgcagaccctcctgtgactaggagttcttagaggaggtcctgggcatgggcactggagtccctcccatgamtagaaattctcagaggagttcctgggcatgggcactggagtcccttctgtgactaggtggtcctgggcatgagtgctggagaccttccagtagctaggagttcttgttgacttgggcgactgcagaacaccacaaggcattgattgattgattgattgattgagatggagtcttgctctgtcgcccaggctggagtgcagtggcgcaatcttggctcactgcaacctccgcctcccgggttcaagtgattctcctgtctcagcctcctgaatagctgggactacaggtgcatgccctcatgcctggctaattttttgtatttttagtagagacagggtttcaccgtgttagccaggatggtcttgctgtcctgaccttgtgatccgcctgcctcggcctccaaaagtcctgggattacaggaatgagccaccgcgcttggccttattttttattctccaagcccaaccaagatttccagacatggtggcctcctgagcac

SEQ ID NO: 42 - DNA, 801 bp, Homo sapiens
acagagcttgacacatccaacaaggatggactttaatttaactcaattaatttttttagaggtagattcttccctatgttgcctgagctggatttgaactcctgggctcatgcaatcctcctgccttggcctcccaaatagccggactacaggtgcacaccaccatgcccagttctagacctaatttgatcaaatgggatgcccatcctgtaaaggctccgccgtggcctggtgtctctcatgtctcagggaggccctaggggctgacccacctcgaaggaagaaggtgtcgtgctggtcacgggctgggtgctgctggggctggaagagggcgtcaaagttccagaaggagctctcaatgaagttatcagtcggcatctcggtgaacctggtgggagacrcagcctgactgccctgcctgtacccagcagaagcaccaggcaccgcccccagccctgtgctcaccccatctccaggaagatctgtcggaactgggagcggaccttgagcagcgggtgaaggtggccgctgtcggggaggacaccgtgggccaagaagttgtagggcttgaagggccggtcccgccaagagccactgggggaggatgcaagggcctggtaagggctgcccgcccctgccccctgccccagctcccacctgccctgaccctggggttgcctgctacctggagatcatctctgggctcagctctgtctcttgcttggagatgctggtactaaaggcactgcctttgctcacccagtaggtcttcagagtcctgtggccaggggaaggaagggg

SEQ ID NO: 43 - DNA, 801 bp, Homo sapiens
cacagttccaagataagatatcatttccccattgttaaatctatgtgcaaagctaacttatgattaaattatggtaagcttaactgtaatacaaggaatcatttcatactatttgtaaagcagtgaacactagcgtaataagttatgatcgtggaggctaccacctgcaccatccataccccgagggctggacactcataactatgggtgaggccattccttccgctggggcaagaataactatagatgcctatgggaaactgatacaatctcattctctacacttcctttatatgctgaaggtcacaatctttattcttgcttagtgaagagagaatcgactgagctacatccacacactattcaccataatgagaattggaatattacttcaaattctkcagtcaccgaagcaaaatctgtaacagggtggatttcaaaaattattttttatcaaggcataacctcatttattttaggttcatggatgtaattttattctccaaggtaaccttatcttcaaattttataggtctcatgctgtcccccatcctgtatcacctacaggtgacttttccttgatatgacttatagaacattaacagtgctttcttttcaatcatactccgatttggttagttccaacttaggggacacttactttaaaatgtctcttcaccctgcaggaaacgagtttattagtgaaactttcaacggatatttttattccatttatgccatttatctctccatgccttgtgaggcaaaggagtggtgtttcaccaggttaagaggtaattg

SEQ ID NO: 44 - DNA, 201 bp, Homo sapiens
tattcactttattgtgatatctgctttattgtggtggtctggaaccaaaccaacagtaactccaagaggtgcctggactaccacaatccaccagcatgcgygaatatcaactgacaagcaatattgaaaaatatagtcacttttctttaatttcttttgaaaagaactaatcatattagaagtaaaaagttattctgaaaa

SEQ ID NO: 45 - DNA, 807 bp, Homo sapiens
ccggtcttgggtggccggaaccacatcctaggtaaggaccctgccctccctgctctcagagccctggggaatgcgcaggtgatgaggaggaaagagttggagccctgggtcccagtcctggctttgtcactgacctgccagggaacccaagggcctcaatttccagatctgcaaaatgggcaccctcggtcttttgtatattatgtctgtcaaacagccgatgcccgacctgagggagaaccaaagcccactcagccagggacccaaaccatgtcagctcatggatgttttgtgtttattttgctcrtctgtggtcctttttaagacaatgccgttaattttggggacaaaatttcttgctctgataatcaaaaataattattccagctaggcacagtgactcacgcctgtaatcccagcgctctgggaggctgaggcagacagatcacctgaggtcaggagttcgagaccggcctggacaacatggtgaaactctgtgtctactaaaaacatagattggacgggtgtggtggtgggcgactgtagtcctagatactccggaggctgaggcaggagaattgcttgaatctgggaggtggagattgcagtgaactgagattgttccactgcactccagcccagaaaacagtgtgagactccatctccaaaataataataataataatttcatcagcggcagaaaattgtaatctgtctaacatgactaagaaaagctcctacctgggggcagggacataaggactcaaagagatggtgcaggagtgctgtgtacagggcctggcgctt

SEQ ID NO: 46 - DNA, 501 bp, Homo sapiens
aatcctcataatgattaaaaagaaaagactaaaaaagaagaggaaaagtagaagatgaattagatacaaaagaatattatattactggcttagtcacttattctaaaacctaccccttccatatggaatattgtaccagggcacctgaatcagatgtgcatagactctgacactatcttgtcttcacttcaccaatggagttcattatgtgagatttgtagtccaaaacaactctcagaagcaaatcttcraaatatgcaagaatttggtttggaatgagttaatttccatgctttggctaaagtagcagaacaagaaatgataggcatagtacagagaatggagaaattaagctcattccagaaagcagcaagctctactagaaagaaaactgagagggtacgaacttcttattttaggctatcaatcgacacagcagggcattttcacctaattttatggattaaggatttgaaaatgaaagcacgctgaagggctagt

SEQ ID NO: 47 - DNA, 1001 bp, Homo sapiens
ggtctcagagagaaatgtgggattccagatgtacacaagcttataaggcatttggggaagccactggaagcttagcagatatagtttcaggttctgaaattatctttgtttaccatttgattcaccctttggtttccaggctcatggagaagctcatgtcttgtatgttcacatcttgtaagaaaagcaccaagccttgcacagtgtaggtgaccaataaatgcaagtcaacactgaaatgtgaaaggactgggagagaggagggggaaagggtaaggagcccaggcgtgaaggcagggaagcccagtggtcagagctggggttggcttcactgaggtgtctgggtggtggtgggtagaaaggtcagtgttgtccagaactgtccacaagctccggctgttctctgtaacctcagtccctgtgtcttcagctctgagcctccctccttgaatgatcctccaagttcctgtcctgacctcaggaggaaaagggatgaaagayagagaaaaggaaaggaaagatagggaggagagaaggcagacacataagagtaagggcaattgagggcaaggacctgaaggatgaagacaggggaacaagagatgccaggggctgcggtccaagaaagcagtcccagagaggggaaagatagaaaacacttgtgccgggcttccgtttacaaaacagtttcctacacagtgcgggcattaatcccactttgtggctgaggaaacggaggctcatagacattaagggtcttgttcaaggggctaagtcagtagtggtgaaggtgggtctcacccaggtgttctcattcctaagcctgtattcgctcttctccccaaacaactccaggaaaggaaaggattgaagacttagggaacaaatgaagtggcttctttgaagcacttgtacaaagaagggtggagaatccagatttttgaaacttttctgcatccagttattgtgtagattcacttagaagaaatggcatcagatgg

SEQ ID NO: 48 - DNA, 4000 bp, Homo sapiens
tgacattcctgaaagagatggagagagaacaagcaacttagaaagtatatttgagaatattgtccatgaaaatgtccccaaagttgctcgagaggtcaacacacctcaggaaattcacagaatgcctgcaagatactatacaagacaatcatccccaagacacatagttatcagattctctaaggtcaatgtgaaagaaaaaaccttaaaggcagctggagagaggggcagatcacatacaaaggaaaccccatcaggctaacagtagacctttcagcagaaacctcacaagtcagaatagattgggagactattcagcatccttaaggaaaagacattccaaccaaagatttcatacctagccaaactaagcttcataagtgaaggagaaataaaatcctttcagacaagcaaatgctaaaggaatttattaccatcaggcctgcctgataagaggtccttaagagagtgctaaacatgaacaaaagaccattatctgccactataataacatacttaagtacatagcccactgacactacaacttaattacacaatcaagtctatacaacaagctgctaacaatatgatgacaagatcaaattatcacatatcaatattaaccttgaaagtaaatgggctaaatgccctccagagtaacaagttgggtaaagaaccaacacccaactgaacactgtcttcaagagacccatctcacatgcaatgatacccataggctcaaagtaaagggatggagaaagatctatcaagcaaaaggaaaacagaaaggagcagggggtactaatcttatttcagataaaacagattttaaaccaacaaggatccaaaaggacaaataagggcattatataatgacaaagggttcaatttaacaagaagacctaactatcctaaatatatatgtgcccaaaaatggagcacccagttcttcatcaaaccagtttttagaggcctacgaagagacttaagcataaaataaaagaggaagacttcaatatcccactgacagtgttaggcagatcattgagacagaaaattaacaaacatatttgggacctaaatttgacaattgaccaaatggacctaacagacatctacagaatactccacctaacaacaacagagtataaactcttctcatctgctcatggcacatactctacaaatgaccatatactcggccataaagcaattctcaacaaattcaaaacaaccaaaattataccaaccatactcttggaccacaatgcaataaaaatagaaatcagtaccaagaagatctctcaagaccacataattacgtggaaattaaataatctgctcctgaatgacttttgggcaaagaatgaaattaaagcaaaaataaaaaagttatttgaaactaatgaaaacaaagatacaacataccagaatttctgggacatagctaaagcagtgttaagtggaaagtttatagcactaaatgcctacatcaattagttagaaagatgtcaaattaacaacctaacattacaactagaggaactagaaaaacaaaaacaaaccaaccccaaagctagcagaagaaatgaaataaacaaaatcagaaataaactgaatgaaattgagatgtgaaatccatacaaaagatcaatgaaaccaaaattcttcaaaagaataaataagattgatagatagctaattagattaataaagaataagagaagattcaaataaacacaatcagaaatgacaaatgtgatattaccattgaccccacagaaatacaaaaaaaaaatccttcagagactaggaacactactatgtacacaaaccagaaaatctagaagaaatgggtgaattcctggaaacacacaacctcccaagatagaaccaggaggaaattgaaatcctgaacagaccaataatgagttctgaaatggaattagtaattaaaaaaaaaatcctatcagccagaaaaagccttggactagatggaytcacagccaaatactagatgtacagagaactggtgtaaaccctactgaaattattccaaaaaagctgaagtggagggactcctccataactcattctctgaggccagcatcattctgataccaaaacctggcagagacaagacaaaaaaagaaaacttcaggccagtattcctgatgatcatagatgcagcaatcctcaacaaaatactagcaaaccaaatctagaagcacatcaaaaagctaatccaccacaaacaagtaggctttattcctgggatgcaaggttggtttaacatatgcaaatcactaaatgtgattcattatataaacaaagctaaaaataaaaaggcttttgataaaattcaacatctcttcatactaaaattcctcaacaaactaaaaatggaagaaacgtacctcaaaagaataaaagccatctatgacaaacccacagccaacatcctactgaatcggcaaaaactggaagcattctccttgagaactagaacaagacaaggatgcccactttcaccattcctatttacatagtactagaagtcccagccagagatatcaggcaagagaaagaaataaaagtcatccaaataggaagagaggaagtcaaacgatttctcttcacagacactattatacctataaaaccctatagcctctgcccaaagcctcctggaacggataaacaacttcactgaagtttcaggatacaaaatcaatgcacaaaaatcagtagcatttctatacacaaataatgtccaacctgagagtcaaataaaaaatgcagtcccattcacaatagccacaaaagaataaaatagttaggaatacagctaaaagaaggaggtgaaatatcctacaagaattacataacaccgctgaaagaaatcagagatgacacaaacaaatgaagaaatggtttgtgctcatagataggaagaatcaatattgttaaaatagccagctgggcggggtggcacacgcctgtaatcccagcactttgggaggcagaggcgggcagatcatgaggtcaggagatcgagaccatcctggctaacgtggtgaaaccccctcgctactaaaaatacaaaaagttagctgggggtggtagcacatgcccgtaatcccagcaacttgagaggctgaggcagaagaatctcttgaactcaggaggcggaggttgtagtgagccaaggttgtgcctctgcactccagcctgggtggcagagcgagactgtctcaaaaaaaaaaaaaaaaaattggccatacgtcctaaagcaatgtacagatttaatgctatttgtgtcaagctaccaaatcatttttcacagaattagaaaaaacgattctaagatttatatggattaaaaaaaaagcctgaatagtcaacgcaatcctcagcaaaaaaaaaaaaaaaaaaaaaaaaaacaaagctgaaggcatcacactacccgactttatactacaaggttacagtaagcaaaatagcatagagctggtacaaaaacagacacatagacaaataaaacaggttagagaacccagaaataaagccacacatctacaaccatctgatctttgacaaagctgacaataacaagcaatggggaaagaactctctattgattaaatggttctgtgataactggctagccatatgcagaagattgaaactaagccccttcattttaccatatatatatatatatatatatatatataaatcaacacaaaatagattagagacttaaatgtaagatctaaaattataaagatcgtaaaagaaaacctaaaaattaccattctggacataggccttgacaaagatttcaagatgaagcctccaaaggcaattgcaacaaatacaaatacagacaagtgagatcaaattaaactaaagagcttctgcacagcaaaagaaactatcaacagaataaacagtcaacctacagaatgggagaaaacattcacaaattatgcatttg

SEQ ID NO: 49 - DNA, 501 bp, Homo sapiens
cctttgggcctcagtctcccacagagagggagagctgggtggcccagcaatctctggagccccttttggctcagacctcctgcagttctctgtagcctcctggttcccagtgtatttttaaaggatcctgtgttatttatctcagcatctctgcccccctgcgctccacgctcctatccatgcagtaatgacatccaaacacccacactgtatcttaacaatctgatgagataaatgcctcatcgttgggytccgtggacacagagtagcaattctattctgctatcaaagagctcagaaactcaggcaaccaaggcaggatgagcaggcatgtggcttgccatgtgcccatcattgggcaaaaacacattctccctgccccaaagcccctgtttttccccaaagcagcctcactttttcccttcctccacctccagaacaatgccttcgagcctgctggcatttgagaatgggccgggcgggcagtactgggggcatatg

SEQ ID NO: 50 - DNA, 1001 bp, Homo sapiens
gcagtaaaaatcatgctcactctacttatagtgttcccagtaattttaaagtttacttttaaatctccataaagacatttttacatttaggtaagtaaaaacttctctcttgtcaaatgaatgtttcctctaatatttagaaagctcagtccacaaataattgctgtcactggatattattgtcctagacctgctggttggcagaagaatcagtctttgacacttgagattttcaaaagagagcagttagtaaatgggaaagaaaagcttatcttcccaactatcttctagaagaagaaaatatctttgaactcatacctttacactgcctgcatatgtcacaggtcctttgtccacctgcgctggagaaactatttggaggacagggactgcaaatctgattcctgttattatcacagaatgtacctaagaaaggcagacaatagtggtacacgtttgacaatgggcaatcgtcacccaaggcattccgaagtttacaakaaaataagattataaaagaatgatgagttttctagatgccacagtggtggacattagtttgattgacttcctaaaacaatacatttgcaagattattctttgtgtaatcacagaattagaaataacaaaatacaggccaggtgcagtggctcacgcctgtaatcacagcactttgggaggccgaagcgggcagatcacctgaggtcagcagttcaagaccagcttggccaacatggtgaaatcctgtctctactaaaaatacaaaaattagccaggcgcagtggtgcacacctgtaatcccagctactcgggaggctgaggcaggagaattgcttgaacctgggaggccaaggttgcagtgagctgagatcgtgccactgcactccagcctgggcgacagagtgagactgtgtctcaaaaaaaaaaaaaaaaacatacacacacaaaaaaaacaaaaaaaccagatattgtaacaaataaatcaacagaaagaaaatact

SEQ ID NO: 51 - DNA, 1001 bp, Homo sapiens
aaaaagataaaataaaaaatttaaaagtgtatttttcttttttaaaaaaaaaaaaaagacaatatgattcaattaaaaattttcgtttcctggagaaataattgtcaagtcaggtggcatacctgaggtttcctctaaaaaattatcactgtcttatgggctttataaatagatataaattgtataaactacaccccccccccagtactatagtacttctgtctattcttaggagaggcttaatattttgcatggtgagaattaatcatattgaataagtaattgaataagcaacaccacattacttacctgcagaccaattacactttggccagcttttaattttccttcatcaaaacgtcttgtttgtttttctgcatacttaactccaatgtcaatggttgtatggaatccttttgttttagcctagacagaaacatgcacactaactgaaaaggccaacagagtttcacagaaggaacaacaaagaaatgatatgtycatagacaaaggggcccaaactagagataaaaagtatttaaggctgttcaataatatactgcatcatttgaaatgtgagtgttctaagtaagctatctagtacgatgcagctgatgtcagcagcctttccacaaagctactaattacagaagagacaggccccgccccacctggtctgaacccccatgaaaaagcaatgatgcaattcatcaccaatgtggtgggaatctgttaaccagccattaaagacatgtacgtaccagacctgctagagccaccagagtagtctgaacctgggtcatgtttccattctcaaaaagatcatttgcttcgaatatgtcatgtggcttcataccataagcctgaatagctttaataaagttgccaatattctccaactataaagaagaagagttttataatttctggagctaaatattgacattcataatttctaagaattccaagtcttcataaacagttataagacgctgcccaga

SEQ ID NO: 52 - DNA, 1001 bp, Homo sapiens
ctccgcgatctgggcctggagctcggcgatctggtcgctgaggtctgtggagtctccctccagcttccggcgggtcttctccagctcctgtcgctgcttctcctccctgcggaggcgctctgcaatgcagggggagcccacatagccctcagtgccatgggttctgtctccgtgtcaattacttatacaaaatactgagcactcgttatgaaacgtgtgtcaggcttaaagcatcacaaacacacagacactcgcacccaactcctggatgcaggaagaggacatcactgcaaactgctagcccctggcatgtctctctccaactcaccccttgtccttccctgccaggaattaagtactctcctaaagctggtgtttgtaattcccttggttcccttgaaggctgtaccatgtctgtatctctaaaaaacatgttcaattgtgtgtgaatctgaacttatataatggaactgccttgtgtgaattccacagttggaatcrcacggtcagggctctatgtctagctttataactctgtgatgtcacttgggaaagctactttctatacctcatctgaataataagcaaaaggagacagcctacttcattaggatgctgtgaggcgccaatgagttactatcttaaaaagcacctagaacagtgcctgcctcgtgatggttattccctgccgtgcctttgccaccccatcacgtgatcttgagatgtagcccgtgaagtttcgctggaatagatgtgtaaggacatttgtctaggattccattatgagaatacaccacaacttacttatgtacccgtcctgcagctggcgggcatgcggctgtgttcatcgcacgctctgatcaacgctgctgcaagcatcctgctcactctcagtgcacacggctgagtgacggatggaacggccaggtcaccggcatgctaagtttcaacctcactaggactgccggatcgttctctaacgccgtctaccagtttgcagt

SEQ ID NO: 53 - DNA, 1001 bp, Homo sapiens
gactgaggagctgcctctccctaattcacaggagcccagaaggctcaaacagatttaaaatatacaaaggagggcatgggagcagcagatcctcccagaccccttgttagtcacaggtactcctggtctggctctcaaaagcaaggccttcccaactttgttacagtcagctatgctgaagccttggctggcagaaactgacaagccccataacacagctttccccatctggccccaaaccatctctccaggctcacctcccctctcaccccattccatctacctccctactgcaacaacacagacactgacaagcagcactctctccagccttccctcttctacaggctgtttcatcttcctgaatgactttgcccctcttctctccccggaaaatccctacttattcttcaaagccccctcaaatgttgcctgatcaggagattactctgattcaagctgagtttgctaccctccactcctatcgagcacagccaacayattattattttattattattactattattgtcaccatcacatccatcatttatcaagtgcttgtcatgtgtcagatactgtattaaatccttcacatgcatttttccttaccacttatagttattatcattctccattttactgaggaggaaaccaacgttcacatcatttcaaatattggcctaaggtcacatagctgctaaagggatttcaaacacagtctgttcttaactcaaaaactcatattgtttccagtaaaatatcacaattcatttatctctgttgcctccaacaacaaataagtaacttaaaaataaagatcacgtcttcctaatttttgtatagcctgcataatacctggcatatagatgatgctctctaaagatttaataaataaatgaataacagacattcattccattttctcaggaacttgaatgacaactgtagcacagaaccagtaatacaatgttgagactgtctttatgtcccctttggct

SEQ ID NO: 54 - DNA, 1001 bp, Homo sapiens
ataacccaggcaccctgtgctgcagagaatgtctgattagttaatattctccagtctgagatctgcagaagggtgtctcccagtctcttccagtcccgatctctcttcttgggtgactacttagttctgagttaaacctggctgtcaaattgtggtgggtacagtcacagaaccatagaatcctagagctctgagggaccctaaacacagaatctagtggcttgaaccttaagatagtgactctcagaggaatactaaagtctacagtgtcctacaaggtcctacatgagctttcttgaccccttccccaccatggccacatcacctctcagaccccatctcctaccactctccacatccctcattctactccagccacactgatttagctgggccttgaacacaccatgcatgcaaccagcctcagggcctttgcactggctcatccctttgcctggaactcatttccaaaatatccgcacatctaactccctcatctcmtttgagtctgttcaaatgccacatttgcaataaggtttaccttgactactccatctaaaattccagccctccctccccatcctctgtgtgtacaggaatctttgttttgttcgctgacccagacactaccttgttcattgtcccaggcacctagaacagtcctagcacatagtaggtcttcaatacatatttctttaatgtataaatatcactagatctgcttcttgatggagaaaagaggaactcattattagtgtgcagggactctgaggtgaggtcactagccaaggttacttagttaaaaaatgccaggggcagggtttgaacccagagcggttgaaatactctctgatgcctattagttctgtgaccttaggcaaccttgctgagactccgtttcttcccttgtaaaatgaggatcggttcatcactccttagagataattgtaagaattaaaggctatgtcactccataaaccttgtcttgtaatttaggtctc

SEQ ID NO: 55 - DNA, 1001 bp, Homo sapiens
acagaactcgggagaggggtggggagagggtgcggggaagaagaggggccaggtgctctgcccacacactgagtctgaggagccatccaggagcagagggctggaaggtgctgggacctggggggctcttggctcaggagggggctgtgctggagcatcgccagcagagggaagccctgggatcggcaggccaaggaggaggcaggcctgggaaaagccctctgccctactctgaccacatggccagggaaaggaccccagcattgcggctgcccccaccagcgctgcagaatctgcttcagaggacttctcgtggcacacagggagaggtgggaggaacatgggaagaacagagagccggcttcagaggccgggcccaggctctggtcggcgggttgctaactccacccccgagaaagacatcctggccctccagccctggggacagagacactcaaacaggtggctgtgctgccatcacatctgagtgtcttcacacaygagcatgatggtggaactctctccccaaggctttctgtcatttccaaagctcctcttgcttgagtgctagtcctgggctgggtgccggggccaagccacgcgtgtgttttcttaatgcacacacccaagctctgaggaatgtctcattgttcctgtcccatgtcacagatgaatagactgggctggggagctccggtgggaagtggagaagccaggattcagggtcaggtaccctcactctcccagtgggccactctgcctcttcctggggcctactcgcccttgggtgtttagcgggggcctgtgatgtgcggagcctggagcctggagctggggaagcagagaggaagccagaccctccccagacaggaaagagacaggactccacagggtggagagggttttctgggaatgggcccagaagcagtctgggatgagggaggactcctttttccctggggctggtggcaggtgggcacccagaagcctgaaattcaagg

SEQ ID NO: 56 - DNA, 1001 bp, Homo sapiens
aagctctgtgccatcctgtcttacggaacaacctggaaagaaaagcagagagaaataatgatgggcaatgacagagcaagattgcagatggggctgggggtgggttctagcccctatggctctggaacctttaggaggttctagaaggttctagagcctttcttcccttggattcctcaagcaacctcagcatccttttccgggtaccaaagacagtatctctgtatccttctggtgcttaagctcctttgagttgtacttttattttttcataaagaatcttgacttacacatctattaagtgacagaactgagtttcaaaccctcatgtgcctaaactgcatagctgcgtctctcagttttctcacctggaaatgggactattctagtacccaccctgctggattattgtgacagttgggtacactgattcagtaaagctctgaaacgaatgcctggcagctaaaggtactcaagttattaagtcagtgttatcatcarctccattttgcagatcagtacactgggacaaaagatgttatggactttgtccaagatcactcacctggcactatttatgagcaaatggcagagccagaaattgaaaccaagcagtcagcagagcctgtcggcttcacgtcgaccctgggagggccctgaagaagtcatctgcccccaccatgcccccgttctgtccagagctgagtgggtgttccgggagcctgggtcaggcttccagggcagaaggactcccagagctggagctggctcctttctcagcttgcttgtgaaagaaccacaggcaggtggggcctcccagctctgagctgcagcggccaggtgagcacagtgctctggggcagtggctcccactgagccagggtcttatgcaaatgcacctgctcccacccacctacccaggtgcctgtggaggcgccaccatatctctctacttgcgctgtctccaagtcgtcttccctgccgtctccaccatatggagg

SEQ ID NO: 57 - DNA, 1240 bp, Homo sapiens
ttttctatcctgtgtcagcctggttcccatctcccccaagaggtaagcactggtgtcaactcatttattcttctaacgattttgtgtagtttcaaacattctcttccttttaaacaaatgacagcattatgcacagtgttctatacctctatgcctttttaaattaaaagagtatttcagaaattatttaatattggttcttctttatagctatataatattccattgtgtggatgtactatgattaacaaatcccctgctaataaacagttaggtttttccctcttttcccttggccaaacaaagcttcagaaaaacatcttaaagcattatgtgtataaatatattagtaagataaatttctagaaatggaattattaactcaaagggtatatacttttttttttttttttgaggtggagtctcgctctgtcgcccaggctggagtgcagtggtgcaatctcggctcactgcaacctccgcctcccgggttcacgcctttctcctgcctcagcctcctgagtagctgggactacaggcgcccgccaccatgtctggctaattttttgtatttttagtagagatggggtttcaccgtgttagccaggatggtctcgatctcctgacgtcatgatccacccacctcggcctcccaaagtgctgggattacaggtgtgagccaccgcacccggcccctcaaagagtatatacatttttaaaattatacagttcctgataatttttctctaggattaaacaatttatagtcctattagcaatgaataagagtaactatctacattctcaccaacaatgtgttatcaagcatataattgtataagtgaaaagtggtatttcggtgtagtttttgatttctatttcttcaatggagggaggacacacatgtttctaggtttataggtcattcatatttccttttctgtgtactctcattcatacattttgtctagttttcttttatgttggtyatattaccctgttaaagctaaacccttactagggtccagaaggccttatgatctggtacccaggtgtgtaaaattttctgtggattagataataccagagaaaatttgtgagagatgataaaaatggaaaaatgatgctaaagcattccaaaggctatggcctgggtgctcacttttgctttaaaaacttccacatactgggaatatttccttgggaaaataatcggatttcatagtggaaattagaagaa

SEQ ID NO: 58 - DNA, 401 bp, Homo sapiens
catcttacttgatttctcaataccagttaacacaattaaacactccctcctcctgaaaaactattttctcactaggcttatgggacaccacactcccttggttctcctcttatcttactggctactcttgcatcatttcctttacctctacttgtctaatgcccaaggacttacttcttggacctctcattttctctatakatactcattctcttggtgatctcatccagtcttgtggctctaaatatctaatatggtttggatgtatgccccctccaaatctcatgttgaaatgtgattcccaatgttgaaggtggggccgatggaaggtggtatatactaacaaatcccacattatcagaacatcctgcctgaacctctatatcaactgcccactagtc

SEQ ID NO: 59 - DNA, 1001 bp, Homo sapiens
atttctgatggtcaatttcagagatttgtaagaatattcaatttcagagattcaaagaattttaaaactaggaggagctacagagactcaaccaaatctcaattccgtagtactttgctatgctgttgtttcctaatgtcacctccctcagtaaaaaggaatatacatgatcaataaatgcgatatctttaggaacacaaagaaaagggataaacgaggaatatataagaaataaagggagggatagcattaggagatatacctaatgttaaatgacgagttaatgggtgcagcacaccaacatggaacatgtatacatatgtaacaaacctgcacgttgtgcacatgtaccctaaaacttaaagtataataaaaataaatcaataaataaaaattaaaaaataaataaataaaaatcaatattcatcagaaaaaaagaaataaaaagcaatctacaaaatgtttaaaataaaaaccctaatctcaaaacaatgcgagaayaatgtcaggcacaagaagcaagaagcataagaaaaactagaaacaaaaaaaaaaaaggtgttgcccaatttgaacaggaaaacaatagataactcaaaaaagctgattcttttaaatcaaaaattagccttaagagattttttagtgttatttaccgagcactattctaagtgctttacacttagatccattacacttcatcttcacaaaaccctcgaaggatggtactattagcgccgctcccatttcctagtgctgaggcgcagggaggttaagttaattagggcagtgctagatctgggtcttgaccccatctgaagccagcacctgcaagcatcactactgctaatacttgccatccattctttaaaagccatccatgttgacccggttgaaaaatttcttctgacctgtgtagacagctttacctttgcataaataaatcacactgtgttttaaacatctatattcaggccttatgtgagtttctttggccaagt

SEQ ID NO: 60 - DNA, 1001 bp, Homo sapiens
agacttaaataaggttataatttgttgagaaaacattttggtccaagaattcacttttttgaatattttaaaaaactgattcagtcggtttgttttcaggaatatttgagcttcatttttttaaattgtgatatcattgaatttctaagaatttccatttttttaaaatagcagaagaactgttttcactttgtttatcaaggaaggcctgaaacacacacacacacatatacttatttaaaaatttctctttctttttgatctattcaatattaatatttagaagacaatttctaagtgtatgcatttacacatatatataattatttaaaaatttctatagactttttgatctattaaatattaatatttagaagacacaaattctaagtatatgcatttacattattgataaatttaattactacataattcatttaaattttattcatgatctaattgacacagactttttgaaattttgttaatatctatcgcatycatgacctagtatttgaacaactttgtgagtgtactttattcttgaggaaacacatttttcgagagtttcagagtgctacatatatatatatcttttaatctagcatgataattattatatacaaatatatataactaccatgctagataattatatatatataatactgtatatatagtattatatacatactaatatttttgtgattttagcttctcaatgtttgaaagaggaaagttgaaatctttcactatggtagcggttttgtttttccctgcagctctatcaacctttgctttatatattttgagattatttaagcagaggctgctggttgtacccccttgcccagcctgttctctacttcaatattataggagctgtacagattaccattgccagcctcctttgcacctatacatgatgttatcactaagatacagctaatggctaatggaatataagtagtgtaatatggtatcttctgaaagttgttaaa

SEQ ID NO: 61 - DNA, 1001 bp, Homo sapiens
aatcccaacactatggaaggccaagttgtgcggatcgcttgagctcaggagtttgagaccagcttggggaacatggcaaaaccccttctctacaaaaaatacaaaaattagccaggtgtggtggcacacgcctgtagtttcagcttcttgggaggctgaggtgggaggatcacttgtgcccgggtggtggaagctgcagtgagcgagatggtgccacagcactcaccctgggtgatacagtgagaccctgtctccccgcaccaaaaaaaagtttattaaaaaaaaagacaaaaaaaaagtcatcaacaatttcctgtcgttgcctaacatacggagtttcatatagaacccagtatcagcaggctctctggccttggtagcagagcagactgaacaggagggaccctccctaggacagctgggaaaagtgcagtctggagcagcgattctccccgacttgggaagggagaagtaaggatggagcctgaacccttgagtacyagcccaaggttctgagatgttcttctccttaaacaaaatcctacacatgctcctgtgttactgaaattcaccatcccaatagagaggtgacttcagcagcagactggagactaattaaggttttaccatcaacatttgtgagaagggggctgggcatggtgggtcatgcctgtaatcccagcactttgcggctgagatgggcagatcacttgaggtcaggagttcgagaccagcctggccaacatggtgaaaccctgtctttactaaaaatacaaaaaattagctgggtgtgttggtgcgtgcttataatcccagctacttaggcggctgggacatgagaattgcttgaatccgggaggtggaggttgcagtgagccgagattgcaccactgcactccagtctgggcgacagagcgagactttgtctcaaaaaaaagaaaaaaaaattgcgtgagggtgaggggaggagatggggttccatgaggcacacacaatgaagt

SEQ ID NO: 62 - DNA, 1001 bp, Homo sapiens
gagaataaaaacattttaggatgatagaaacaatgtatagtgaaataaattggtacaagtaaatgtttattgaaaatttatggccaaattacaagtggtgactcttaaaataatttgcatgcctaacatagcctatgcacaaatgtgtcacatttttttcttttcttttatattatattgaagggttatttgtgagaacatccacctcatagttgggagccctccctggaaatttgtatccctcaaccttgatattctaatttctacaatgaaatctccagaaacagcttcatatggttcaatgaagaggtttgcaattcagggtaacttgtgggccattatttttccagtaaagaaagaaaaaaatgaagaatataatatattaaccttattgtacagagtagctggaatattaagaagtaacctctgaccttaatttattgataagggcttgcctttgagttaaagaattttgtagacctaaaatatacacagaaatcrgggcttaagaaagcctggcaatctgataaaattaagtaaaatttcttttgctgaatggacagaatcatgaattagttggttgagcgtgtccacaatataacttttcttccgtgagagaattttggaggttggcttggcagttaataaaatttgctttagagatgagaatcagacattgcaaaggtcagacagaattcctcagcagattctgcagtcatgaaaaattcagccattcctcttgatttcctccctcgcacattgtatgaacatttccgcttgctggcaggtgtgtgcctggggctgggtattggacccacctgattatttcctggtgtatctcgtcctttccacttagctccctgctgcctattctagaagcagcatacacagatgcatgagccagtgtctgtggcttactgctgagccattctcttctaaataccaccagtagtcccattccaactgagagccaaagataggttaaaatagagtattttcag

SEQ ID NO: 63 - DNA, 401 bp, Homo sapiens
gccatttatgccaggatggagaaagaagccttattacagtgagaaaatctcaaatctcagtctttgggcaaacggaaacagaaaccagcaattgggctgcacacatttcccaggagggacaaagtctatgtaaggtcatttttggtctctgcttgcctccactttaggaaaaatgccgctgccaaaccctttgactctatyaatgcctcagctttcagagttgatagctaaacctatatctctaattatctttttataagtctcctctttcacatgtatctccaacccaggcagcatggtgaaacatacagttcataataagggaatgtggactcccttgagcatttcagggtgaaggcgcactgtgggtgttaaaagggaaaatatttaaagatcagtgg

SEQ ID NO: 64 - DNA, 1001 bp, Homo sapiens
cagcagcaactgtagcctcctagacttgttcccgcttctccaaaactgcgtgcttagaatccctgagctggaagagcgtccagaggggaagagaggtcttaagcattccagacaaataaagatctgttccagccctaacaggctccaaggagagatgactgcagcttgtctgtcattcatgtcaggactcccctcaatgcagcagctataactatgctcaggtcttcctgctgcagttctggctcatttcagttctcactccgccactttcgcgcccttcactttcccactccatcaattaaatctttaatgatagctaatcttggtggctctttcacctataagactgctgtttgtaatgaaaatctgtaccattcgttgacactttattatattcagtttatagactattgtcatatatactaacaagggcatacataggaacttcatcttaacatttctcccatagcacctagcacagtgccaattctgttagaacayagtagttgcttagtaaatattggatgggcagatggatggatgaatggatgggtggatagatggataaactgacagcaataaataaatgagtaaactattaactgctcgtagccaggtgtggtggtgtgcacttgtaatctcacctactcgtgaggcagaggtgggaggatcacttgagcccaggagtccaagaccagcctaggcaacatagcaagacctcatctcaacatgtatgtataactattaactgatctctaaataatccatgcaatcatgttatttccttagctaacctaaaataagcttataaaggtaaagaccatgtcctgcacattatttgtaacctcagcacctagccatgcttagtacatagtaggctcacaagaaatgcctcctgagttaactaaatttccactcaggctgagctggaatgctgactgccccccaaccaagccttgtagcacttggtttgggcaagaagggaatgctgggtaggatgc

SEQ ID NO: 65 - DNA, 1001 bp, Homo sapiens
agaagatccaaataaaagtaggacaaaagaatagagtcagaaaatctcagtaagctgccctatttgctaatactattccaaaaaaaaaaaaaaatcaacagaagaggatgttctatggaattttaaaaaaaaaactagcctgttccacaaatttttctgtaatttcaaaagaaaataagttatcaaaactatcacagacacatcccgagcaaagctattatgagataaataagaataaggagtagaataacatccatacagacagtaaaatgatgccagaaagtcaggctgaccaagcagatcaaattttatctatttcaaaatgagttaaaagactttgattaaatgacataagataggaaagaacaacaaaactcaaacttataaaaattcagaaatgaagtgacagaactcagtaaagaattagaaataaaagaaaaaaaattaataaagactattagaagacatataagaaaaattaagcacaatagataatgcagyaagagaaatagaaggggaaaagaagaaaaatattttaaaaccaaaaaagaagtgtaaaaagagataagaagatttaaatagaaagtgacaaatattgaagataagcaaggaaaatccaaaatacaggtaatagaaatccctgaagaagaaaaccaaagcaaagacatataacaaatactaaaactgcaattcaagaaaactaaaattaaaatataagaagatgggaggccaaagtgggtggatctcctgaggccaggagttcgagaccagcctgaccaacatggcaaaaccctgtctctactaaaaaacacaaaaattagctgggcatggtggcgcccacctgtagtcccagctactcgggaggctgagacaggagaatcgcttgaacctgggaggcagaggttgcagtgagccaagatcacaccactgcattccagcctgggcaacacagtgagactctgtctccaaaaaatgaaaataagaattaaaaaatatatgaa

SEQ ID NO: 66 - DNA, 1001 bp, Homo sapiens
aattaaccatcacaggaacaaacaaagtcatataaacaatattagctcaaattctcatgctcttgttaacacttacatcacaggatccgaaataattgccaaggcttctttttcaaaatatttttgagagttctgaaggttatactcaaagagaagctctgagatgctgctttactctttctcatggggcagcaaaatctgcatctgggctcttcaaatgtcaaagctgtagagtgggctctggtaaaatgggccaatatttctcaaccccttttgtgtaagaaatcccaaggcaatgttgcttttctatcaagattttaaaatgtctttagtaaaaaaagtggttaagtctttgtttacttttccttctagtcccagacacaagcaattagccttgagtgaactcatatgccagccaccactctgaactcaggaccactgctttccacaacgaccaaggctttgttgggaagtacagcccatcatgactggctttgtctstgcttattaggaccatctaggggatttcagagttccccgctcaatgaaacacctagtgattcattcatgattcacccccatcagttctccactcatgtttacacagtctgaatcccagtttagattgtgttgtagtccaactacattagttattctgcttgtggtggtgaattttaggggtcagcttgattaacgaatgctcacatagctgctaaggcattattcctgggttgtctatgagggtgtttccagaagagactggcatttacatcagtggactaagtggtgaagattcaccctcaatgcgggtggacagcaaccaatcagctgaggtcccagatggaacaaaaaggcaagggaaaggcaaataatgttttctctggctgttaagagaaatccacagaggtaaagcaccgcattcgttggaaagctcagtaaatagcagtatttacacagttcttcatataagcttttaaaaagcaggtcacatagccagacta

SEQ ID NO: 67 - DNA, 868 bp, Homo sapiens
ctgtgacccacaggtggtcacagagaccacctctgggcataggagaaatgggtagatgttatagggagactggttttggccagtatcttaatcagctctggttgctataacaaaatacccctgggtggcttaaacactggatatttatttctcacaattctggagtctgggaagtctgagatgaaggtgccagtggatttggttcctggtgagggctctcttcctacttgcagacagccaccttcttgctgtgtcctcacgtggtggagagagggaacaagcttgctggtgtctcctcgtcttcttatatggacactaatccgttgctgaggccccaccttcatgacctcatgtaaacccttgatttcccaaaggccccacctaacaccatcccactgtgggttacccttcaatatatgaatttcacaggacacaaacattctgtccagagcatccagtattggaaaagacagaggaacaatcctctgagaacagacaccrccttgcgccctttcctctggtgttccaatggcaggaaccctctgggaggagtctgagaatgggtgggaagtggttctccttccaaacatgagtttctgtgaccagggctggaactacggtgaggcaatgaaatgcccagagtgcaggatttaaggcagcactcacactcaggggttgctcctgggattacaaaagcctgaaggtcaggggggggcctccttaaaatttgcacctggcaccttgtgtgtctcaccctaatcctggccctgtccatgactcagtcttcaaactgccccattaaagatctcttccaatcaggaaaacataaaaaggtcttttcctaggcagttagttcagacacagcagg

SEQ ID NO: 68 - DNA, 1001 bp, Homo sapiens
taacagacaacctccaaaccaaggcacacattttgccttgaaaaagattaattgccaagaagaatcatctgcatttcagagtagccccatgtttgatttctgccagttgccaacctgacagtacttgcattaatcctgtcagatatcaaaggtgagttacagtcactatggagaaagaatctgtgtcagtacaagataatttgtcacgctgattctgcagtgaagaatgattagagttctgggtagagcaattttcctggggatatctgaatctatatctacagctgaatgtagcaaagagtttcttcaacatagggtttaaaaaatccttttcacagagatgtccgaagtattctgttcttcttttcaaaaacctttaataactttttcttacctacagaataatagaattgtatgagatcacctaacatctgaagggagaccagggtaagattaacccagaagaaacctaagaaggaagacaggagaagagggatagarttttatcttgaagccaatggagaagagcatttcaaaatagaagggttggtaaacagtgccaaattcttagggttaggaatctgttgggaaggagtttaagaagacttttgatctagatgattacataaattgttactttgacagatttttatctgctcctaacacacagtaatgacagataaaatatgaaagaagaaaattcaaaagatacatttaggctaaaagatacagtcactgaaatgaaaaaacgaaaacaaaagcaaaaatcctcaataggtataatgagttatagttttaacacagttaaaaagagcattagaaatttggaagatagtatagaataattaacccagaaaatagaacaaaaacataaagagattcaaaaatatgaaaaaggtgttaagagacacagcaagtagatttatgattttccatatttttaagaggagttccaaagaaagagactgaataaaatgctagatattttaagaggtatttga

SEQ ID NO: 69 - DNA, 1001 bp, Homo sapiens
cacctccagcccaaggaccccggagggaccctgccaagcagccacaccctgccaggctggggagggcttccctccttcctggaacagggtctcaatttttacaggaggaagcaatttgcagagcccactaggagggaggtgggggcccctccactctctccccaccccccgccctgcttattaatgtgcttccaatttgggactggagattctctgcccctcctccccacactacttgggaaaggggctttctcagggagcagagggggttgggatagtccccccaggcctccttctgcaaagctcatctcatctgttgtcacgctcctgctccctgaagcagtatagcgggtaggagggtggagactcagccctcaagtcccctgtagagagaagggcccagtttccagacctcccttcaccccctccctgggactgacttatctcctcatcttgtcctggtcccagggtgggaatctcaggtatccagtgagagacagrctatagggggtcactgggtttctcagttctcagttaccatgccagactcctggtactctgggtcgcccactgggctcccaccagccactcagtcagtgccctcatgagagtcacggtgtcatctaagaacaaataggtttcctctgcgcccttcctctcgctcgcatacacagttgcgtcacagccactgtcctgctcattcactgttacccgcatctctctcctgctgggtttttaaaaatgtctttattgtgataaaatatacataacatcaagtttaccattttagccatttctaagtgtacaattgagaggcattaattacattccttctttgtttttatcagtgtctttctcattgtcttccacatagatgccacacgtcagggaccacaggcccccaaaagcatcccacagagcctcctacacgccctgcctggcacagccctgggggtgtcacccacactacccgagagtcccaaacacgctcagatagtgac

SEQ ID NO: 70 - DNA, 1001 bp, Homo sapiens
tcaagataactaagataatacatagtgatatgacatagtgatggtgaggatacaaactggattactccatattgctgactggaatgtaaaatggcacaaccattctggaaaacaatttggcagttttttaaaacactttgcatgccataattttgtgactcagcaattacacctctggccgtttatcccagatgattaaaaactttcattcacacaaaacctgtacatgaatgttcaaagcagctttattcgtaataaccaagaagcagaattgtcctgatgtccttcagagcaagaatggttaaacaaaccgtggtccatacatactatagaataatactcaataatttaaaaaataaattattcatgtacacatctgggtgaatctccagggaattaagctgagtgattaaagtcaatagaaaggtaatatactatatgattcaatttgtataacattttcaaaaatgaaggaaattttgtgtagaggacaaattagtsattgtcagaagttagagaagggaccagtcagggggactagagagactagagagaagcaggcatggttacaaaacagcaacattgactgtggtaatgaattgggagcctacacatatgatatagaaaattctgtagaatgtaacactcacacacacatatagacaaatgagtataagtataattggggaaatctgaataaaatgggtggatcatctttaggtgaatatactttttatgatatcacactgttgttttacaaaatattagaattgggaaaagctgggcaaagcatataatggaactctctgtttcttcttagagctgtatgtcatctataattttatcaataaaattttccatttaaaaatttaaaaatagaaatgcaggcatagtttcctgttaaagtcagctgtgtgaaatatatcatcataatttaggagagagagtgcaaagtcatgtgagagcacatgtgtgtgcatgcatgcattcacacagaggga

SEQ ID NO: 71 - DNA, 1001 bp, Homo sapiens
ggctttttttttttttttttttgtatttttagtagagatgggctttcaccatattgggcaggctggtcacgaactcctgacctcaagtgatcttcctacctcggcctcccaaagtgctgagattacaggtgtgagccactgcacctggcccctgctttgttttagattttacaaagctctctctggttgctgtgtgctgaatacaccctagaggacaaggaagtcgtcactagttagaaggctgttagatgctgtaggtgagagatgatgatggcttccacccggttggacatgattgaggtggtaaaaagtggtcagattatgggtgtatttgaaggcagaaatgatcgtttcttctggtagattggatgtggtgtttgagaaaaagggagtcatggatgacttcaggatatttggcctgagtgactgcaaatgtgaaattgacatattctgagatgaagaagaatgcaggtggagcaggtttgagttgggaggagagcvggaatcctgtttgggaatgtgttgaaagtgtgtattagatgtccaagtagaggcgaaagaagcagttgatgtccgaggttgcaatgcaccagtgctggggcgcggtggggatgtaaagcaggaagcactggaagtgacagcttaatgatcaactcccccacatcccacatagttatcaggaaaaggaaaatgagggggaaatcacatgttaaatcagaaaggagtggtcgggtgcagtggctcatgcctgtaatcctagtactttgagaggccgaagcaggtggatcacctgaggtcaggagttcaagaccagcctggccaacatggtgaaaccctgtctctactaaaccctgtctctactaaaaaatacaaaaattagccaggtgtggtggtgggtacctgtaatcccagctactcggaaggctgaggcagggagaagcacttgaacctgggaggtgaaggttgcagtgagctgagatggtgccattgcactccacctc

SEQ ID NO: 72 - DNA, 5361 bp, Homo sapiens; identical to SEQ ID NO: 34

SEQ ID NO: 73 - DNA, 631 bp, Homo sapiens
ctgcctatcagtgaagactacttaacaaaaagatactcaccctccgacttatcctttcattaattgagaaggaaacatgaatagaatcgtatttttaaaattggggaattgacgactctttaaaaaaaaaattaggtagaccaaataaactggtcttcctcagtttctcccattcctgcttctacctaataaaataattargctcactgcaacctccgcttcccaggttcaagtgattctcctgcctcagcctcccaagtagctgggattacaggcacctgccaccacgcctggctaatttttgtatttttagtagagacagggtatcgccatgttggccaggctggtcttgaactcctgacctcaggtgaactgctcaccttggcctcccaaagtgctgggattacaggcatgagccaccacacccggccccaggaacccttttcccaagccaacacacaagtgctgagtagatactgcccaactaaattacctggacatatctaagcatgggcctgagcaccttttaaaaaaaatcttggcatatatttgtctctaaagattactttagtgatacgagcaaagatttttatactttcttaaaaagcatccatttttttaggtcaaaaat

SEQ ID NO: 74 - DNA, 1034 bp, Homo sapiens
tttcagtagtttacatactccccatggtaccacgtagatccaatccacatttttccaagatggatctggaaaaaattcaatttccgcatcctggagaatacctagttctcctttcttgttctcctggtacttgattaagcgagttgtataatattgcacagcaaaaaaatcagcagtgcctttgatcattttcttctcttcttcagtgaattctggaagcctcgatgatggatagccttgcttttgactcatggaggcaatctgagacttgacaacttcaggataatcaccatcgatgaatatgggtttagcaaataaatccagatggaaagtgatggctcttttagcagcttcctggtcagacactgagttgggatctgctggttccaaccagaccgcaaaaagtgatagagacaccatacctttctgcttttttcgaaataaggaatcatagctgtgccaggatctggcatgagccttaatcaaattatgagctgcctgataacctccagtcccaaagtgagggatacccggaggaaacatacctaagtcatatgacatcacagaaagaacattagcttcatttatggtgatccactgcttgacacgatccccaaaggtactgaagcaaaactgagcatatttgtcaaaggattcaatgattgcctctgacaaccaacctccttggtcttctaaagtctgaggcaaatcaaagtggtagagggtcacaatgggagtaaccccattttttaacaaatcatcgatgatcttgttgtaataatcaattcctaggaggaaaaagaaagtgccctaagaagaaaagttatttgtagtatattggcaataaattttaaaagggagaatatcaaaaacaattttctgaatgacttttattcaaaacaagttttactacttccttgatataatcaatgaaagactgagaggaaacaytgagaattccaaagaagagtagttgatcaggaagtaggagtttaatggggtgacatgattgaaagataaacgtattgaaatgg

SEQ ID NO: 75 - DNA, 1001 bp, Homo sapiens
ctgcccacgatcgttgggcaggcttcaaggtttcctaatcactattggtctgaatgcctgccagtcacaaagaattcaaaagaaaggattggcccaaagggtaggggcgggaaaaggttagtgcagtcctgccttcgcacaatggctattggctgatacggtctaagtcaatgtgcaatgccaagggattggtaataactcgctacacgctgtcgcctggccaaggagggctttattcgtctgagtagttgtcagtcataaccaaagccataagcaatttgctcgggactacctatagacctcgcccactataagcccctttctttccttcgcttcctcttttagagaatgtccggattgctattggactttggagcgtatggctccaaatcaactcattggctaaaacttgacggaaaatggtggttaggtaaaacgcgcctgcgcagcacgcggcgggacgggggtgggccaatcctgtgagggtttaaccttctcttkttccacctcttcacccctatcttgtcgccatggtgactgctctacaattggcgaggcttgcacttcaaagtcctaggctcgcttcatccgggtccttcagctgtggactttctgctgattgggccttttccttttcccctgattggccgacatcgggaaagacggcgaagagctaggaaaagagggaaaacactagggtcgcagggttcaaaatggctccaacctcctttggtgacgtagagagcagaacttgggtctgcccctcccttttagttaagggagcagaactgggattagcccgacgtttggatagtgggaacatcgatctgcggcgctggtgttaacccaactcattcggctggacgactcagccctccccatattaggtgatttacagagcaaaactgaactaaaggcccacccctttcttaatgttgtacacagagtagaacaggattgacttcaactccgttttaaaccttcagagcaggaaagctctg

SEQ ID NO: 76 - DNA, 401 bp, Homo sapiens
ttacaatgatactatttttagtgaggagaaatttattaaaatatacaaatgtttgaggtaacgttaggtattcaggacaaaaaatgtttattgttttgattatacatgcagtaccttttgtgatagtataaagagctatacagttccatatcagcacctgaaagtagttagtaaagaaggatgaggctcatggccatcgawtctggattagaagactggatgggctgttagaacggttctttagtgcaaatgattttgctgcccaagaagggcagaggacaggtgggggaagtgagatttaggtagtactttttctctaaccttcataacaattctatgaggtggatgttattattcctgctgtactatggaagaaattaaggcagagagagggcaaataa

SEQ ID NO: 77 - DNA, 1001 bp, Homo sapiens
acccccaagagtttcagggaagggactgtgggcctatgtagtgactcaattaactctcccattagccctgttgacagggaaacttgatgttcccactaaaatgatggaggcagagtgagtagcagcctgaggtcatacagctaaggacacgccaaagcctgtactgacacttgagaagtttcttgatgttccgtcctagaatctcggggttggcgggacctcaaaggccagagagtctggccttcccatccagtgtttgggttccctgtgctgctggtgtcctagacaaactgccatgcagcctgtgcttgagcacccagaccaactggggcaggcgccaggcaccaggcaggcatgcaatggtggtcagccgccgacagactcagagctagatccccagagggatgggacctacagagaccccagaggctaaaggaccgcaggagggcggaggagtgggggatccagagagtcccagagacaggagggcccaccctgagyaccaccagtcaactcccaggcacatggcgcatacccaccggggcacgaaagagtcggttttctgcaaggtggtggggtccagctcaaagagagaggcgatgtcattgccatgaggcacagccaccaggcagtacttgatcttctccccagcattcctgcggcctgtgcggccctgtgccccctggatgaggaccccaaatcaggtagaaggccctccctcttctcaagcccatgaagcacctgggatcccacctgagatcccatctgtctccctccagcccacctcccaggttttcacggcctcccctgcccccgcctacccccagggcaggcccatggtctcactggagacctgctttgctcctcaccattcctgctgccttgggatctgaggcatcacctttgtaactggggttctcctggacagagatacaagatgtgggggagctactggacaccaaactatacccacacgacccactgcaaaacatggtcaccta

SEQ ID NO: 78 - DNA, 1001 bp, Homo sapiens
ttgttccttcctcattccgacatctctgtataactcggaggagagtgaaataagatggagaagtggtggacattctacttttccttgagacactcctatagatgatatagtaagtctaagatgctctctgggacaaaaggggagatgagtagggattgcctagcaaaaaaattctaagtggaagaggcactctgaatcagcaccacttgcatgttgtgtgaactgatggccagggacatagaacattagcccctggttgaaggctctgtcttcctcttctacctattgggtacccatcctcctcctctgcaccctattccccaccatcttgataggtgatcctttactcaaggttttcctaaaacccctctttgccatacttttgcatcctcctgtttcaaaaggaagaatgaccttggactccatggcacttgtcagcatcttgcagtgaatcagatccttcctgaagtggtataaaaaggaaggtggagaggaaaaacwtctggcctgattttatgtgactatgcccccttctcaccattcaccaccaatgactgcacactcaacatactccagatcctgaaccaatcttgtgccttgtacaggacttttaaagagcctttgcaacattctacccacccttttgtttgggctgcccatgagctatgatttcatttctctaatgccagtgagctgcttatcgccatattctctgttttccatttgtgtgcctggactccctccttttatgtagagccacgtccacgtttctcccttctgcagtctttgctacatcagttctctagtacctaagggtgtactgcataaatatgaggagctcagtgcatgctgagtgaatgagcaaagtttaggatgagggcatgagtaaatgaatgaatgtgtgagggattgaatgactatctctgaaactgcactgaacccacaggtaggttacatcacaggacagaaatctgaggagctggagaaagcaaaagaataaa

SEQ ID NO: 79 - DNA, 1001 bp, Homo sapiens
cctacgcatattttaaaccttttttgctgcgtcctatatgtctcttacgttcttgtctgtgttttctatcttttttctctccatgcttcggtctggatattttctttgatattcttgactaattcttaaagactatgtcatctactgctaaatctagccattgaattttaatgacagttattgtagtcatcatttttaaaaatacttgccagtttggcgctgaaatttcccatcatttcgttgagttcctttaatatttgaaccgtggttatttaaagatccacatctggtaactccggtgtctggctgatctcctgcgggccggtttccatgatctggttgtaggagtgtgcccctgtggctgtgacccatcccaggttgagaggtagccgtccatccaggtgcatcaacacacatttccacgtgtgctgtttcaagtcatttcggcccatgttaatttctcctcgtattataatcaggttgtttcttattggtttataytttcaaaacatgccatttcaatttaatgaatctttgctgaggacaaccttgaagctcaaagtggttaaaagatttttcccaatatccctacagcattcagtgactacagaggactttcgttatgttttctggagctcaacatctcagagcccttctgatgtttgggaacagcagtccgggctaggacactctcagcacaagcacatgaccctctcctgtccaaccacatgctccctctaggctttgcatctggcgggagtgatgcagagatgtaggggaacccttggaattcgtgccaatggcagtggtggtggaggcagtggcaggtcctgcagtggcagcagcatcctgcccacgccagactcacaccctgtgtgaatttggccactgtggtgaccacatagcctcccttggttctggctactccctttcaattctgtgagttacttgatacccttccaatacattcctgctttgcataaactaaccagagtggattt

SEQ ID NO: 80 - DNA, 1001 bp, Homo sapiens
cgcgtcccttccccgacctgccccaggcggacgcggtgacgtgtgttggcctcgaggctggaatacaccggggatcaagtgcagagaagggagaaagtagggaaggatggctggggggtgggggtggggggagcgtgttgaagaaaaaagggaagagagaggaaggaaagaggagaaaaaaggtgaagaagagaataacatttaaaatatagagttttattatttctaacttttatttttggtttttatctagttttggtatgtatgaatattcttaacatagctttatctctgtctctctctctgaatctgtaaatatacagtaatatatatacacgtaagcctctacctgccgatgtgtcagggtgtgtctcttgggcacaaaaacaaggtttttgttttgttttgttttacataagcaaagtacaaatctcaaagaagatatattttaaaagccattttattgggacttgctttgcatacaataaaatgtatctaaamtgtatctatttgaaatgcatagctcgttgtgttttggctgttgtacacacccacatctccactaccacaatgaagatgtagaacatttccatcggactccaaagagctgctatgcaatacaattttatagggtcaataaaagaggtaagatcagttttaagtattgttatgagaagatgtgtgcgtctcatacttttaaccattttttaaaagatgaggatacactgaattataatgccagtaataccacttccataatgtatattttaagtagggaaaaacctggaagatttctcaccaaagttttatttatttatttattttttgagacagagtctagctctgtcgcccaggctggagtgcagtggcgagatctcggctcactgcaagctccgcctcctgggttcacgccattctcctgggttcacgccatcctcctgcctcagcctcccgagtagctgggactacagacgcccgccaccacactaattttttgtatt

SEQ ID NO: 81 - DNA, 1001 bp, Homo sapiens
attgagtgaaagtccagccacacccttggtgttcctttcagaatatgtttttgcattttttgcaatatcaataggctgaattttccaaatatttaagatctagttccttgttgcttaacaatcctttcttcagttcatctctgtcctctcacatattactataaacagtcaggaggaactaagatactccttcaacactttgcttagaaatattctcagggaaatttccagtttcattgcttaccagttctactttccaaaaaaacactagaacacaaacacaattcagtcaggttatttccctctttataacaaggattgcctctccttcaggttcctacatgatctttgttaccatctgagacttcacaagaatcacccttaatatctatatttatagtatgcaccttaaaactcttccagcctctacccattacccatttccaaagcaacttccacatgtttaagcatttgttacagcagcacctcacttctcagtaycaaaatctgtattagtctgggttatccatagaaacagaactgctaagagatataaatagatagatataaagaagcttataagaaattgggccaggtgaagtggctcacacctgtaatcccagcactttgagaggtcgaggtgggcggatcacttgaggtcaggagttcaagaccagcctggtcaacatggtgaaaccccatctctactaaaaatacaaaagttagctgtggcattcatctctggtcccagctactcaggaggctgaggcaggagaatcacttgaacctgggaggcagaggttgcagtgagccaagatcatgccactgcactccagcctaggcaacagagtaagactcaaaaaaaaaaaaagaaattgggtcatgtgattatgaagacagatatcccaagatctatagtcagcacaaaggacagccaatggtatgagtaccagtctgagtatcagtccaaagtctgacatcctagcttgacaatcattagg

SEQ ID NO: 82 - DNA, 401 bp, Homo sapiens
aatgaaaatctgttgtctgggtgctctgaggctgtttcccaggcaattctcacaattatacctgaggacacccctctgaggctctgtttgcactttgtcattctaatgggtcagaggcatatctaatcacaggaaaaataacattgcccaatataataatgagagaagatattcttttccaatggcagttgtgctcagacrcattttaactctcctgcctcagggagcatttcctatatatgaacttcttccacagaaaggggaagactttgtatctttgtttttgtgggccatactgtttctattcctgatcttgttcacaggttatcataatccttaaactcttttaggtcttttgcactaagtttgaaatcctcttcaacaccacaggaatgctgctc

SEQ ID NO: 83 - DNA, 986 bp, Homo sapiens
gtgttaatgttaaaatggagatcataagactgacaaaatggactctttatggctataagatactaaattgtaaacaagacctaaggtcacgccaagcaagagttaagtcacatacccccgtacttaaagaataaactatgttccacctgccacaagatttttctttttctctagcagctaaacaagcactggcctcgagataagaaatattgaaacaattacagctcaccaattgccagatgcagcctgatttcacccctctgttccacaagccatcactacagctctgatgtgacaagaggttgattttggtaactttctcttgatgaaagaccaccaaccatggactggttctggctggtttacagaggctgcgcacttgaatgcctctgtgtctctgcttcaccttttgacatacaaggcctaattgtaatgcatttatatgttaagtctccacacaaaagtgaacatgcaacatgcatgtttattcagtgcacacgygttaggacacccttcatgaatatttatagctactcttataacctgctaaatatttatacttggctaacctgttcagcataaatccctgttccatcctcccctccctcaatgtacctgccttctggcttctaccagaggctacgtttcccagcctgtcaaaatgaccactctgcaggctgtaaccctttatgagaaatcaactctccattctaaatgtaacatctcataatgtttcagctgacacttcagagctgcctctttttaggaatgcctgacatccagacctacatggttcttggctttagcaatggagcacgtagtacctaaataagtcaatcaaagccacagtgattggttcagtggtgggcatgtgaccgaacttgcttagattctgggacaaagactctttcttttaatctggatgtggtggtatgtgaatgagatgcctggaatccttgtggccattttgttaacatgaaatagcc

SEQ ID NO: 84 - DNA, 1001 bp, Homo sapiens
tctatctacagacaacagcatgtgcatccatcagggcacctgactcattgcctctattccaaggcaattcctgactggattaggttcaaaagagcaggtagagaaaatatgatactatcagagagccttgggcctttttgttaccctggcaggttctagctagattatcaccatcggtgttccagataacaagacactccttttctctgttaaagctcatgggagactttctttcaaacagacaggaaattgagaatcctggatagttactctccagccctcttccctcctgtccctggcttctcattccagagaagttcctgccccacatccaggaggaagaaatgctgctcagaggccatattagaatccaaacagacctttctgggtttacctattcagtctattagtgttagatcacaccctttttgtccagtcatatttctacatggctgtgcatacttggctgaatgtaagcattaaaatggaccatttcccctrtctctttggcccttcattctgaaggctcctatgtcatttaacactatgaccaaataaatttgtatgccttttctcctattaacctgactcttgttagtgattttcagcaaacctacagaggcctaaggaaaagcttgcccttggccactacattactctcaggccaatttatccaactccttctaggtaaactagagataacaatgcctactttgtagattttcatgagttttaaaaaacttaacaagactttattttctttctatctctctttcttctactttgagccttcagtttctttctttccagacttactagaattcatttcaattgaattctggatgtttgcagaagtcaggtcattttcataggatgaagatttaccactattgagttacttaatttaaaaataccacagattttgtttgtttgtttgtttgtttgtttgtttttgagacaggttctcgctctatcatccaggctggagtgctgtgacacta

SEQ ID NO: 85 - DNA, 801 bp, Homo sapiens
agccgggggcttaccctgcgtggctcccagcggcgaacgtgtcaggaaggtggctcttggagcgggacggagccttcctgccaaggtgacctttgacctgtacccccaggtcagatcctggtcttccatcctactgtcttctctccccacctcaaccctgctctttcctcactttgtttaaacctccctgtacaactatctcacttctgagccttttataccctggaaacccatgatcccccgtctctttggtcactgtatccctgacactcccagacatttgacctcatttctgactctcccagactccttcatgtacgacacccctcaagaggtggccgaagctttcctgtcttccctgacagagaccatagaaggagtcgatgctgaggatgggcacrgcccaggtttgaagacagagaagggaggcagggcagggaactgggggaaaatggagaagggacagaactgttaatgctggagcctgagccactctcctggcacccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggaatcctcctatccctgaactcgggggaatggaatctcgctgatcttccaggactagctccctgatcattccagcccctctgaacaacagggccccaggaaaatctccaggtcctattctgtcctccttcccttttacttgaagcagtttctt

SEQ ID NO: 86 - DNA, 1001 bp, Homo sapiens
cttcattgccaatgactgagcatctgtctctggttcacaggtcatccagcttctttgttcattttctttagatccagctggctccctgatcccagagcatagtctttccctgaggctcgctactcaaaagagtcaaacttcatccagccctcacttcttccacccgctcttcaactggtccaatccactttccatcctggatactccactgactgcaaataccaactcctccaaacccagtacttgcgtctctgtcacgttcttacttcactcacctgtcagtggttctcaccacaactggccactccctcgcctcgaaaaaatcatttttctttgattcccatgcatcacattccttgggttttttttctccagcatctctggggaatcttctcagtcccttatgctgtcctgtggccctctgatattttttctacacaaaaatctatctccctctgcaacctcttccacttccctggaatttaacacagaacctgcatygaccccaacataaatacctccagccctggcctcaccctgaactcctctcttatattcagttgacttcctgattgcttcatgtgagttcaaaaatcatctcaattttaataaacacaattgtcatttctaatcaaccacttcaaatcatttcctcccatattcttccctatttcaataagcagcaccaccatccacctatttatcaaggcaaaatacttagaaataagttacatttaatccattaacaagacatgcaaaaagacatcccaagtctgttcactttatctggatctgtctttgtcactactacactacatgaagccaaaaatttgtcttccctggagaattctgctgttctccacttgtgaaccccaacaatccaatctccacatagtagctagaattatttttaaaattgaatattatcgggggacctgcccggataatcacgtagtttcttttctattttcctaagcatcggccggcttgagaaataaagg

SEQ ID NO: 87 - DNA, 801 bp, Homo sapiens
tttacacaacacctttggaaaaaaaatctagacaaaaatacaccatattacatcaagatgttcattctttctcatctggtatttctcctgggactatcacataagaacataataaacaccatgaatataaaaaaaaaaaaacaaaataagaaagcctacatacctaatggttaagaagataatgatgcctctactccattaagcattataaaggcactcaaattatgtgtattaagagttttattgactcggagaaatgtttatactataatctgatactaagttaaaaaaaaaatcagccctgggaggatgccaggttatccacataggaagcaaaagacatcacagatttcctttaggagcctggagaatttggactcctgctcacaattactgactckatgttttctgttgtgtttccaatggtctaattaagtttctcttcagataatgctttggccaagctcttttttgttacaaatacacaatataaagatttcactctgcattccccgttaccatccttctgcatcagtaccacacacaattaagttgatcatgttagaggttctatgcttaatttttacacgctgaagaattattaggattttaataagtcacagcacttaacatctattaacgagtattgaaactcataaactattcaattttctaccacagtgtagccatttttcccattaaaaagcaagaacaacagaaatcaagtcaaagaacaacagaaaagtgctattgacaggaaaagcctcacctagaccacatctcgctttttagaattgatgc

SEQ ID NO: 88 - DNA, 1001 bp, Homo sapiens
ggcaaacaaactgcctcctctacaaatgggtaatcatatcatctactgaaacgtacactttggagcccagcgtggtttttgctgtctgaatcaggggttcggtgtcctttatgtcaacaaggacgctatcgctgatcttgtccaggtgttcaatagcaacacgagcagcctgctcatagccatcggctattctgattgggtgaatgcctcggtctagcaattgctccgcttcttctaacaaggcaccagccaggactgcaaataagccagcagacaatcagttcaggcaaacaacaaaacttcacttttagtataatcagcacaagttagatgctatcatggaaagaagcagcttctagaattaagctgcaagtcatcttagaggcagaagccctctatttttaaaagaaagcctcgttttagttctagcgaaggtcgaataggacatgtgtccccttacagatgaacattcaaccctcagtgatgtgaagaacgtaactraagaatataatgtaaaaagaacatttttctaagaggtaaaaagctattatgtttcctgggccagggtctactcagtgaaattcagcctggtgatgagactaaaacgtgtttattatttattccccccacccccatcctcctttctcctcattttagggtcgcaaagatgaattttgtttaaatctacagctctccaagcgcaccgagaacagtgcttggcatacaatagtatccaataaatatttgttgaatgaattcaaatttcatctgcagaaaaggtaaccttactgatattttgtcttcaatctccccaactttttaaagatttcaaatcttcagaaaaaaaaggtacaatactaaaacgaataccagtttacttattaacattttgccacattttctctacatgtacatactgtttctgttcaactatttgagaattagttacagaaaccatgacatgtcactcctaagtatttaggtatacagctgagaagaag

SEQ ID NO: 89 - DNA, 1001 bp, Homo sapiens
ctgcgtgtgtgtgtgtgtgtgtgtgtgtgtatagaatgcatgtcgttaccaaatacctagaacggtattggaccgcaaagaggttaaacccggttgctttcgacaggaatgagttgaaagcacccaagcgttttcgggtgggtatcgccgtccatccattcgatggcctcgctgatgagcatagaagatgaacccgggaggactgtcccttcctggctgcgaaacctgaccgatataggggagattctagaagaaagagctaaggaggccagggcggaaagaggaaagtcaggtgtaggggagattaaaaggtaaactggtgggactgaccgggctggtgggactagttagcgaagccaagtgtttgggtagaaacagagatcttgtaggaggtgctccgagctacatttcgggatttgggatgaaaagagaaagccagggggttcgccgctcctggtttgttccttaccgaccctaaactgtatggcggagcttgtgggkcaggcgtggaggcggcatccactcccacgtgccccacatgcagcagcaatcccacgcggttttcgcaagcggctgggaggcaagcgggcgtccccccacaccccccccccactgcgccctggacccacgccacgcaccgccaaggagtagctatggtggcacgtggaggaggggcgggtaggcagggcggcaccatcaccgggaccagcatcttggaacggtcaatctcactgctcatactctcctccctaccggcgcgggagccttgctgtaatcccctgccaccccatctccgactgtgaggggcgcaccgggtgggggaatgggtaaataactggcccaaggcccagcttccaggtcctccctccactttcccgcggcctgactgcgccccgggcaacacagccccttgggaacctgcgaccaggtaatgtggcctccgccccgtcccgggtctggctgcatcccctctaacccctgcctgcccgggtccaagtgag

SEQ ID NO: 90 - DNA, 1001 bp, Homo sapiens
gaagctcttcccggaagagcttcctggagagaaggggaacgagccagcgtttattgagcatctattatactaagcatctgcttggcagttcacgacggtcgcattttttcatccttacagcgatccctattgtgtcgccttgctttaaagccttacagctcacaaagggctgggatttattccagatctctctctcagatgccatctcacttccaggtgtctctgctgctttgcaacgcgggaaacccacgcaaaggagtgatttccaaggccttctgtttggaatatctttaatcctccccttattaactggaaaaactcccacgcatccttcagggctcagctcaaatgtcctttatctctgcagtgaaactttcccaaggaaaattagttacacagctaattttagataaattgagccagttgatagaatttgtcatgtttagactccttctgtgtttgagttctctccccgacccccaaccaaattgtaggacgttrtctttatgagcctggcattcagtaggctttaaaattaatatctttctgggttgcagtgattccaagcataccatcgttctgtccaccaaaccagaaacaagaagtaatcacacatcttcctaatatgcccagataggcttaattattgttcccaatggagaaaatttacagttaaacatcaggtttcccttagggtgatcatgatcatctatctttctttctttctttccttccttccttctttttttttttttttttttttgacagtctcactctgtcacccaggctggagtgcaatggcacaatcttggctcactggaacctccgcctcctgggttcaagagattctcctgcctcagcctccctagtggctgggattacaggcgtgcgccaccacgcctggctaatttctgtatttttagtagagacagggtttcaccatgttggccaggctggtctcgaacttctgacctcaggtgatccgcacgtcttagcctccc

SEQ ID NO: 91 - DNA, 1001 bp, Homo sapiens
tgtaagtgaaaggaaggtctgtaactttggcctaaggagtgcattggagaaaattggtaggttcctgaggaaattcagtttttagagcaagctcactttttagggcaagctcaccttggtagatgagagttgagtgctggaagtggtaaaataaccaaagcaaactgctcatttcaacattcctttctcctcgcttccgacttgcagtgttccagcttcctgaactcaagctagtacagctaatgctacaagtttagctgcttgatacatgaaagtgatttataatcaggccaagaaaccaacctaccagatgtagctttacttttagagagcatgtatgtatttcaagttctatgcaactgacaaatgaacttttggatgcaacttgttcaaaagtggaagtattgaattgtttatggtgaccaacagtccacacattaatgactgctataattatgtttcttatgatatcaaaactgacatggtcccctaatacagaaygcagttcataatgtggatttggggagtgccagtgttcaccatttgtgtttttgaaacacaaggtataggtggtgtgaggaaaactatcttctgactttaagctactattttttaaatggtatgacaactttatgttgatttcttttggttaatccgaccagtcttagtgttgcatgaaaatgatcttcatcatttccatttcatagcaactcttttatgccttaaacattttttttctgttcataccattcaaaagaagctaagatttgtggtgtgagccaacacaaatcctccctcaggtgctttaataacagtaagacaggcttccttgaatatcctaaatgaaagctttgttttgttgcacttttgaacaaatacgtgaagagaatgatttatgtaatgaaattccatgttttaagagtctaaagaggattggatttatggttcttccacccccatccccaccattgaaactagaaaacttgtgactttggcccaga

SEQ ID NO: 92 - DNA, 401 bp, Homo sapiens
gaagtccaaccctcacccctcctcctgccttgtctaggatcttcagcattagaaagattgctggccggttaccataacacccagggcccttggtggctgctctgtgggatcagccaatcctcgcagccactcacctggcctgcgcatggtgaggcacaccctttggattcacctggtccagcagctgctgccttctcgctrcttctttcttcctacagcgcccctgaagaaggttgtttctgagggtcctgaagagaagttttgtattctgcattgctgacatctgctgaaagggccaacatttttaaaaagcgtcagatcagtctacgtgtggtcttccttccttacttggatggactggccaccatgaaagtttttggaattatttctcttaagtcctc

SEQ ID NO: 93 - DNA, 811 bp, Homo sapiens
ctgggttacttcccttttcaaatactcagtaacattcttgtttgcaataagacagaataagcaccacttattttgttccagatgatgtattgcactgtaagcaaaaatgcttctggtttttatttttaaagtcttccttatttttccccctacagtgctcattgagggcaggatttggcccccagctccttctggaacccaatttccttccatgttccctatcacaaccctacagacactggcctccttgctgttctcaaatactcttgacacatccttgctcagagactttgctgttcccacatttggaattctctgccctgagatgtttgaatgtctgccttcctgctcatgcagctgctgcccaaaggtcacctcttcagatagcctttgcctccaccttctctcccacttatctcaatcctgctcaattgttcttcccagctgttgtcactatttaccattacaccacacattaacacatgttgtttatcatctggmtcccttgccagaacacaggccctgtgaggatatgggaacttttctgtttgattcccaccatgtgcctggcatgaagtatttccttttaaatacttatcaaatgacggaatttcaactccaaggagaacctagaggcctagtgtagataaaaatgaacatagtagattttagctggtttaagaaactaatccaaccttcacttgaggatcgtaactcagagaaggaagacaggcattctttaatcgatagattgcttcagatgcaaagaggcaagtaaaggaaagaagttggttaaaaaataaaataaagt

SEQ ID NO: 94 - DNA, 401 bp, Homo sapiens
atcttaatttctgaaaaagaaaacatgtatgcataggaggtgcaagggagtgagccaagatgttttcagtggctatctcaggaggactccgggggcttcattttcctctttggagtgtaatcgtttttctccatttcccaccatgaatagatgtgaaatcagaaaagcaaataacgtaggtgtcttaaggaacttgatggwccagctgggaaaacaaagcacataaaagagcagccagtgtcaagtgcagcggaggctggcaagcaccgccagcagaggaggaggcctggatggcagagatgctgctgctgcccaagtggcagagccaagtccaacctcgaacctctgggttctgctgtctgtcccaggggtttcgtagagatggtgggacttggaaagat

SEQ ID NO: 95 - DNA, 1001 bp, Homo sapiens
aacccaagccccagcttaagcccaagccaccagtggcagctaagccggtgatacccagaaaaccagctgttccccccaaagcgggcccggctgaagctgtggctgggcagcagaagccgcaggagcagatccaagccatggacgagatggacatcttgcagtacatccaggaccacgatacaccagcccaggccgcccccagcctcttctgacccttccatgctggcccctggcccagcaggcctgtctgtggggacatcggtgtgaagggaagggactgggccctgcagggtcagaacctccccacccccaggggaggccaggcagaagcctgggtcacagcacccagaactgcatggttccattttctccggggctgtggggccaaagtagaagcctgcgggctgcgggagcggctctcaccctaggagccagagcccaatgtgtcttattccccgtggacatgaaggggagggagggtgtggggatgccttgccaacyagaagcccagccccaaggatgaagcaagacatgtggggccgtagcgaggtgtcacatggggcagggaagcttcatgcccacgggttctgccagccccagcacagacccaaactggggctgggcctctatccctcctctgcctctgttcgcatagtaagaaggagtgaccggtatcctccccttcccctaccctaagctgtagcctgggtgactgactggcctgggcctggggtggggacgtccccaagccaaattactccagggcctctgctcctcgtggctgccaggggcctgcagggtctgggtgggtctcccaggagaggaatactgagtgggagatcggctgtctggagtgttctgatgcaagtctctctctcctgagcctcctcttgatgcaagctctaaagggagaagtcaggccctgcctctccagggtatagacggccctgctaggccccagttcttcctccttccccctttcccaggaaaggccagcccag

SEQ ID NO: 96 - DNA, 1001 bp, Homo sapiens
ctctgttgcccaggctggattgcagaggctaggtcttggctcactgtaacctctgcctgccaggttcaagtgattctcttgcttcagcctccagattagctggaattacaggcacctgccaccatgcccagctaatttttgtattttcagtagagacagggtttcaccatgttggctaggctggtcctgaactcctgacctcatgatatgcctgcctcagcctcccaaagtgctgggattacaggggtgagccaccgtgcccgacctttgatccctttggttaaagtaaatttcttgctcttaacatgaaaaaatatttacaattgcctctgatttaaagcatcaggaactgaaagcatttaagttaaatcctttgggatcctatttccatctctaatcaagacaggtgactttcctccaggaatacttgagtggtaatcaccctgaccagctgcccatgtaaggaattgctaatgattctaaaccatggggcaaatacaragctttatataagtggtaattactgttattgttctctgacatccttacatttcaagctgagttccttccagcaaatgtagtacaggagaaaattaaagctctggtgacatcatcctcataataaagaggagtcactggccagagatgctcacttgtgtttctgaggccattgagagaggatcttatcatcattttgactggagagatgaaaacttgggggtttgtcttcttttgagttgttatagggtctttcctaatcgtgatagaatatttgttttggtcagaaatccctcactgggggaactcccttctattgccaagaaaaaaggccactggctgcagtgactcacttgggactattggcaccatattggggtgcacatgtagctccggacactccagctctcctacaaaacttcctgttctctctcgggaaaaataggaaggctgatccgatccagagcctccatggggggttcaacctgctgtgggtgtggctg

SEQ ID NO: 97 - DNA, 401 bp, Homo sapiens
ctacagtccccaggctgcaccttatcctgtgaggtgtgaaacaagagctggctgggccgaagtagcaagggacccaagtctgacacctggatggacttccagctggctgggagggtagcacaggcacctgatctgagaaacttacatacataaaggagagagagctttatgtttgagggaagttggaggaaattagggttrgagacgggaagggaaaattgaagttcaatgggggttgtttctgaagcttcttcacgtggttcttcattgaagccccagcatcgcccaactaggaaggcacagcaggtgcttggtgtgaagcagtacacctgtgtgacgtgcagcacccactccgcctctgtggggcctcaggtgggctcctttactgtggggcaaatgca

SEQ ID NO: 98 - DNA, 1001 bp, Homo sapiens
cctgtccatgcggccaacggctctgtccccttggagcctcatgccaggctcagcatggccagtgctccctgcggccaggcaggactgcacctgcgggacagggctgacggcacacctgggggcagggcctgagcctacagggaggcacagggcaggtgggctagccatgaacagaagaggaagctggagtgctttgggggttcatgcatgtaggctgggatttggggctcacacctcaacctgcatgcccagttccatgcccctcccctcttgtgaaagcacctgtctacttgggctgaggatgtgggggcacaggtggcaggtgaggctgccctcaggaggggcccaggcccagcttgtaccccacctccaccagtacctgaagaagtggggctctcaccctacctgcctctgccattggaatggcctggtttgcacagatgggaaacccgtttgcggggtgggtgtctgggtgggcacgtggggcgaggacctgcctgmgggaccctgccctggaactgacagtgcaagctcggcgtcctgcccatctgggcagaaggctggtttctcccatcaacgaagccctcccaggaccttcctgcaagccctcgtcccacacgcagctctgccgtcccttggtgtccctcccggcctcaggtcctccatgctgggtacctctgggcacctcgtttggctgagccaggggttcagcctggcagggcgccctggcagcagtccttggcctgtggatgctgtcctggcccgtggatggtgtcccggcctccacgtacccctctcagcccctcctcttggactccagccatgggcctgcgcgcgagccggaactgctccaggacagagaacgccgtgtgtggctgcagcccaggccacttctgcatcgtccaggacggggaccactgcgccgcgtgccgcgcttacgccacctccagcccgggccagagggtgcagaagggaggtaagcggtgggtggcggacacccc

SEQ ID NO: 99 - DNA, 121 bp, Homo sapiens
  misc_feature (81)..(81): n is a, c, g, or t
gccttctgcccagatgggcaggacgccgagcttgcactgtcagttccagggcagggtcccrcaggcaggtcctcgccccangtgcccacccagacacccaccccncaaacgggtttcccat